(19) United States
(12) Patent Application Publication
(10) Pub. No.: US 2023/0250430 A1
(43) Pub. Date: Aug. 10, 2023

(54) CLEAVAGE-AMPLIFICATION BIOSENSOR AND METHODS OF USE THEREOF

(71) Applicant: McMaster University, Hamilton (CA)

(72) Inventors: John Brennan, Dundas (CA); Yingfu Li, Dundas (CA); Jimmy Gu, Mississauga (CA)

(21) Appl. No.: 18/010,878

(22) PCT Filed: Jun. 15, 2021
(86) PCT No.: PCT/CA2021/050818

§ 371 Date: Dec. 16, 2022

Related U.S. Application Data
(60) Provisional application No. 63/039,518, filed on Jun. 16, 2020.
(60) Provisional application No. 63/169,082, filed on Mar. 31, 2021.

(51) Int. Cl. C12N 15/113; C12Q 1/6825; C12Q 1/70; C12Q 1/6883

(52) U.S. Cl. CPC C12N 15/1131; C12Q 1/6825; C12Q 1/701; C12Q 1/6883; C12Q 2600/118

(57) ABSTRACT

This disclosure relates to recognition moieties, biosensors, biosensor systems and kits thereof, and the methods for their use in detecting a target nucleic acid molecule in a test sample, including viral RNA and methods for determining whether a subject has a viral infection. The methods disclosed herein include detecting a viral infection in a subject comprising testing a sample from the subject for the presence of a target nucleic acid using a biosensor system, wherein presence of a target nucleic acid indicates that the subject has a viral infection.

### RELATED APPLICATIONS

This application claims the benefit of 35 U.S.C. § 119 based on the priority of U.S. Provisional Patent Application Nos. 63/039,518, filed Jun. 16, 2020; and 63/169,082, filed Mar. 31, 2021; each of these applications being incorporated herein in their entirety by reference.

### SEQUENCE LISTING

This application incorporates by reference the Sequence Listing submitted in Computer Readable Form as file P61956PC00 Sequence Listing_ST25.txt created on Jun. 15, 2021 (95,998 bytes).

### FIELD

The present disclosure relates to biosensors, and in particular to biosensors and methods for detecting analytes.

### BACKGROUND

Given the rapid emergence of various infectious disease pandemics, point-of-care tests (POCTs) have gained significant interest due to their applicability in clinical decision making for rapid, simple, and early screening, diagnosis, and treatment monitoring.

For example, there is an urgent need to increase the COVID-19 (caused by the SARS-CoV-2 virus) testing capability around the world. However, nearly all approved molecular tests for this virus are designed to detect viral RNA using RT (reverse transcriptase) followed by either polymerase chain reaction (RT-PCR),^{[1]} or isothermal techniques, such as loop-mediated isothermal amplification (RT-LAMP in Abbott ID NOW^{[2]}), all of which use specific primers and RT to amplify DNA from viral RNA. These methods require substantial technical expertise and advanced equipment to perform; most are slow (requiring 1-6 h for the test alone as well as additional time for shipping samples to testing facilities with suitable biosafety containment, data analysis, and test result turn around); and several have registered a significant number of false positives and negatives.^{[3]} Finally, none of these tests are suitable for self-testing at home or in remote locations with limited access to central testing labs.

Thus, only those patients with advanced symptoms are tested, resulting in substantial underreporting of the true case load as well significant potential for community spread by asymptomatic carriers. Undoubtedly, this low testing rate has resulted in substantial underreporting of the true case load, allowing asymptomatic carriers to further spread the virus. New test platforms are therefore needed that do not compete for the resources used in current tests, offer a shorter test time, and are simple and cost-effective to allow for self-testing, such as POCTs.

The background herein is included solely to explain the context of the disclosure. This is not to be taken as an admission that any of the material referred to was published, known, or part of the common general knowledge as of the priority date.

### SUMMARY

The present inventors disclose recognition moieties, biosensors, biosensor systems and kits for detection of a coronavirus such as SARS-CoV-2. In accordance with an aspect of the present disclosure, there is a recognition moiety comprising a catalytic nucleic acid,

wherein the recognition moiety recognizes a target nucleic acid and cleaves the target nucleic acid upon contact to produce a cleavage fragment that acts as a primer for rolling circle amplification (RCA) to generate single-stranded nucleic acid molecules; and

wherein the target nucleic acid is from SARS-CoV-2.

In some embodiments, the catalytic nucleic acid acts as a circular DNA template for performing RCA. In some embodiments, the catalytic nucleic acid comprises a nucleic acid molecule having a sequence as set forth in at least one of SEQ ID NO: 10-15, 17-19, 21, 22, 24, 25, 27, 28, 30, 31, 33, 34, 36, 37, 39, 40, 42, 43, 45, 46, 48, 49, 51, 63-96 and 105-295. In some embodiments, the catalytic nucleic acid comprises a nucleic acid molecule having a sequence as set forth in at least one of SEQ ID NO: 16, 20, 23, 26, 29, 32, 41, 72, 76, 80, 81, 86-93, 95, 96, 106-109, 111-117, 119-126, 129, 130, 131, 133, 135, 137, 139, 143, 145, 146, 148, 149, 151, 156-160, 162, 164-168, 176, 179, and 181-193. In some embodiments, the catalytic nucleic acid comprises a nucleic acid molecule having a sequence as set forth in at least one of SEQ ID NO: 17-19, 21, 22, 66, 80, 81, 91, 92, 96, 109, 123, 112, 114, 130, 139, 145, 151, 160, 179, 182, 203, 215, 230, 236, 249, 259, 262, 266, 268, and 284. In some embodiments, the catalytic nucleic acid comprises a nucleic acid molecule having a sequence as set forth in SEQ ID NO: 80. In some embodiments, the target nucleic acid has a sequence as set forth in at least one of SEQ ID NO: 1-9, 97-104, and 296-307. In some embodiments, target nucleic acid has a sequence as set forth in at least one of SEQ ID NO: 97-104, 296-300, 302, and 303. In some embodiments, the catalytic nucleic acid comprises a nucleic acid molecule having a sequence as set forth in SEQ ID NO: 80, and the target nucleic acid has a sequence as set forth in SEQ ID NO: 98.

In accordance with an aspect of the present disclosure, there is also provided a biosensor for detecting a target nucleic acid comprising:

a) a recognition moiety comprising a catalytic nucleic acid;

b) a polynucleotide kinase or phosphatase; and

c) reagents for performing rolling circle amplification (RCA);

wherein the recognition moiety cleaves the target nucleic acid to produce a cleavage fragment and the polynucleotide kinase or phosphatase removes cyclic phosphate from the cleavage fragment, producing a dephosphorylated cleavage fragment that acts as a primer for RCA to generate single-stranded nucleic acid molecules.

In some embodiments, the reagents for performing RCA comprise a DNA polymerase and deoxyribonucleoside triphosphates. In some embodiments, the catalytic nucleic acid acts as a circular DNA template for performing rolling circle amplification (RCA) or the reagents for performing RCA further comprise a circular DNA template. In some embodiments, the recognition moiety comprises a nuclease. In some embodiments, the nuclease is a ribonuclease, optionally, RNase I.

In some embodiments, the reagents for performing RCA are comprised in a stabilized composition. In some embodiments, the recognition moiety is comprised in a stabilized composition. In some embodiments, the stabilized composition comprises a stabilizing matrix. In some embodiments, the stabilizing matrix comprises pullulan. In some embodiments, the biosensor further comprises lysis agents. In some embodiments, the lysis agents comprise non-denaturing detergents. In some embodiments, the biosensor further comprises a reporter moiety comprising a detectable label that generates a fluorescent, colorimetric, electrochemical, surface plasmon resonance, spectroscopic, or radioactive signal. In some embodiments, the detectable label generates a fluorescent signal.

In some embodiments, the target nucleic acid comprises RNA. In some embodiments, the target nucleic acid is from a pathogen. In some embodiments, the target nucleic acid is from a virus. In some embodiments, the virus is a coronavirus. In some embodiments, the coronavirus is SARS-CoV-2. In some embodiments, the recognition moiety comprises nucleic acid molecule having a sequence as set forth in at least one of SEQ ID NO: 10-15, 17-19, 21, 22, 24, 25, 27, 28, 30, 31, 33, 34, 36, 37, 39, 40, 42, 43, 45, 46, 48, 49, 51, 63-96, and 105-295. In some embodiments, the recognition moiety comprises a nucleic acid molecule having a sequence as set forth in at least one of SEQ ID NO: 16, 20, 23, 26, 29, 32, 41, 72, 76, 80, 81, 86-93, 95, 96, 106-109,111-117,119-126,129,130, 131, 133, 135, 137, 139, 143, 145, 146, 148, 149, 151, 156-160, 162, 164-168, 176, 179, and 181-193. In some embodiments, the recognition moiety comprises a nucleic acid molecule having a sequence as set forth in at least one of SEQ ID NO: 17-19, 21, 22, 66, 80, 81, 91, 92, 96, 109, 123, 112, 114, 130, 139, 145, 151, 160, 179, 182, 203, 215, 230, 236, 249, 259, 262, 266, 268, and 284. In some embodiments, the recognition moiety comprises a nucleic acid molecule having a sequence as set forth in SEQ ID NO: 80. In some embodiments, the target nucleic acid has a sequence as set forth in at least one of SEQ ID NO: 1-9, 97-104, and 296-307. In some embodiments, the target nucleic acid has a sequence as set forth in at least one of SEQ ID NO: 97-104, 296-300, 302, and 303. In some embodiments, the recognition moiety comprises a nucleic acid molecule having a sequence as set forth in SEQ ID NO: 80, and the target nucleic acid has a sequence as set forth in SEQ ID NO: 98.

In some embodiments, the biosensor further comprises a lateral flow device for detecting the target nucleic acid. In some embodiments, the biosensor is for use in for screening, diagnostics, and/or health monitoring.

In accordance with an aspect of the present disclosure, there is also provided a biosensor system for detecting a target nucleic acid comprising

a) a biosensor of described herein;

b) a single-stranded oligonucleotide comprising a first domain and a second domain, wherein the single-stranded oligonucleotide is sequestered by a partially complementary oligonucleotide prior to RCA;

c) a reporter moiety complementary to the first domain of the single-stranded oligonucleotide;

d) a capture probe complementary to the second domain of the single-stranded oligonucleotide; and

e) a solid support comprising the capture probe.

In some embodiments, the single-stranded oligonucleotide is partially hybridized to a second single-stranded oligonucleotide complementary to repeating segments of the single-stranded nucleic acid molecules. In some embodiments, the second single-stranded oligonucleotide preferentially hybridizes to the repeating segments of the single-stranded nucleic acid molecules. In some embodiments, the single-stranded oligonucleotide is generated by cleaving a repeating segment of the single-stranded nucleic acid molecules. In some embodiments, the single-stranded nucleic acid molecules are cleaved by a nicking enzyme. In some embodiments, the solid support comprises a lateral flow test strip.

In some embodiments, the reporter moiety is disposed on a conjugate pad on the lateral flow test strip. In some embodiments, the capture probe is immobilized on the lateral flow test strip in a visualization area. In some embodiments, the single-stranded oligonucleotide hybridizes to the reporter moiety and the capture probe upon flowing up the lateral flow test strip.

In some embodiments, the target nucleic acid comprises RNA. In some embodiments, the biosensor system further comprises an aptamer for detecting a non-nucleic acid target in a sample. In some embodiments, the detecting a non-nucleic acid target in a sample triggers RCA to generate single-stranded nucleic acid molecules. In some embodiments, the non-nucleic acid target comprises protein. In some embodiments, the non-nucleic acid target is from a pathogen. In some embodiments, the non-nucleic acid target is from a virus. In some embodiments, wherein the virus is a coronavirus. In some embodiments, the coronavirus is SARS-CoV-2.

In some embodiments, the aptamer further comprises a nucleic acid assembly comprising a primer for RCA. In some embodiments, binding of the aptamer to the non-nucleic acid target releases the primer for RCA to generate single-stranded nucleic acid molecules. In some embodiments, the biosensor system is for use in screening, diagnostics, and/or health monitoring.

In accordance with an aspect of the present disclosure, there is also provided a method of detecting the presence of a target nucleic acid in a sample, comprising:

a) contacting a biosensor or a biosensor system described herein with the sample in a solution, allowing for production of an RCA product; and

b) detecting single-stranded nucleic acid molecules generated from RCA;

wherein detection of the single-stranded nucleic acid molecules in b) indicates presence of the target nucleic acid in the sample.

In accordance with an aspect of the present disclosure, there is also provided a method for detecting the presence of a target nucleic acid in a sample, comprising:

a) contacting the sample with a recognition moiety, wherein the recognition moiety cleaves the target nucleic acid to produce a cleavage fragment;

b) removing cyclic phosphate from the cleavage fragment with a polynucleotide kinase or phosphatase;

c) performing rolling circle amplification (RCA) on the cleavage fragment under conditions to generate single-stranded nucleic acid molecules; and detecting the single-stranded nucleic acid molecules generated in c);

wherein detection of the single-stranded nucleic acid molecules in d) indicates presence of the target nucleic acid in the sample.

In some embodiments, the method further comprises contacting the sample with lysis agents prior to contacting the sample with the recognition moiety. In some embodiments, detection of the single-stranded nucleic acid molecules is indicated by a fluorescent, colorimetric, electrochemical, surface plasmon resonance, spectroscopic, or radioactive signal. In some embodiments, detection of the single-stranded nucleic acid molecules is indicated by a fluorescent signal. In some embodiments, an increase in the fluorescence signal indicates presence of the target nucleic acid in the sample.

In some embodiments, detection of the single-stranded nucleic acid molecules comprises:

a) providing a first single-stranded oligonucleotide partially hybridized to a second single-stranded oligonucleotide prior to RCA;

b) preferentially hybridizing the second single-stranded oligonucleotide to repeating segments of the single-stranded nucleic acid molecules produced from the RCA, displacing the first single-stranded oligonucleotide;

c) hybridizing a first domain of the first single-stranded oligonucleotide to a reporter moiety, wherein the reporter moiety is disposed near a first end of lateral flow test strip;

d) flowing the reporter moiety hybridized to the first domain of the first single-stranded oligonucleotide from a first end of the lateral flow test strip towards a second end of the lateral flow test strip; and

e) hybridizing a second domain of the first single-stranded oligonucleotide to a capture probe, wherein the capture probe is immobilized on the lateral flow test strip in a visualization area.

In some embodiments, detection of the single-stranded nucleic acid molecules comprises:

a) cleaving a repeating segment of the single-stranded nucleic acid molecules to generate a single-stranded oligonucleotide;

b) hybridizing a first domain of the single-stranded oligonucleotide to a reporter moiety, wherein the reporter moiety is disposed near a first end of lateral flow test strip;

c) flowing the reporter moiety hybridized to the first domain of the single-stranded oligonucleotide from a first end of the lateral flow test strip towards a second end of the lateral flow test strip; and

d) hybridizing a second domain of the single-stranded oligonucleotide to a capture probe, wherein the capture probe is immobilized on the lateral flow test strip in a visualization area.

In accordance with an aspect of the present disclosure, there is also provided a method of detecting a viral infection in a subject comprising testing a sample from the subject for the presence of a target nucleic acid using a biosensor described herein, wherein presence of a target nucleic acid indicates that the subject has a viral infection.

In accordance with an aspect of the present disclosure, there is also provided a method of detecting a viral infection in a subject comprising testing a sample from the subject for the presence of a target nucleic acid using a biosensor system described herein, wherein presence of a target nucleic acid indicates that the subject has a viral infection.

In accordance with an aspect of the present disclosure, there is also provided is a use of a biosensor described herein to determine the presence of the target nucleic acid in the sample.

In accordance with an aspect of the present disclosure, there is also provided is a use of a biosensor system described herein to determine the presence of the target nucleic acid in the sample.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DRAWINGS

Certain embodiments of the disclosure will now be described in greater detail with reference to the attached drawings in which:

FIG. 1A shows a schematic of sample collection in a vial containing processing reagents for viral lysis and subsequent RNA excision, to which the sample is added, in an exemplary embodiment of the disclosure.

FIG. 1B shows a schematic of RNA excision by the DNAzyme in which the viral RNA is digested into RNA fragments and treated with polynucleotide kinase (PNK) to facilitate rolling circle amplification (RCA) in an exemplary embodiment of the disclosure.

FIG. 1C shows a schematic of using the RNA fragment excised in the sample collection vial as a primer for rolling circle amplification (RCA), in a vial containing all the necessary reagents for RCA (Phi29 DNA polymerase (Phi29DP), circular DNA template (CDT) and deoxyribonucleotide triphosphates (dNTPs) to yield the RCA product (RCAP) which contains n repeating units in an exemplary embodiment of the disclosure.

FIG. 1D shows cleavage of SARS-CoV-2 N1 nucleocapsid RNA (n1 RNA) by the DNAzyme at a specific G-U junction using polyacrylamide gel electrophoresis (PAGE) in an exemplary embodiment of the disclosure.

FIG. 1E shows detection of RCAP generated from RCA of n1 RNA in the presence of the necessary RCA reagents in an exemplary embodiment of the disclosure.

FIG. 1F shows detection of the RCAP by fluorescence using a DNA binding dye in an exemplary embodiment of the i.

FIG. 2A shows a schematic of site-directed trans-state DNAzyme cleavage of RNA to generate an RNA primer for RCA in an exemplary embodiment of the disclosure.

FIG. 2B shows an alternative scheme for circular-state DNAzyme mediated generation of RNA primers using a DNAzyme embedded within a circular RCA template in an exemplary embodiment of the disclosure.

FIG. 2C shows site-specific cleavage of n1 RNA by 10-23 DNAzyme (GU1c) using storage phosphor 10% urea denaturing PAGE in an exemplary embodiment of the disclosure.

FIG. 2D shows one-tube sequential DNAzyme, PNK and Phi29DP reactions using n1 RNA in a fluorescence image of 1% TAE agarose with 1×SYBR™ Safe gel stain where RCAP is observed when n1 RNA is in the presence of the DNAzyme, PNK and Phi29DP in an exemplary embodiment of the disclosure.

FIG. 3A shows resolution of 5′ cleavage fragments from screening DNAzyme cleavage of 5′ labelled 32P-RNA nucleocapsid full substrate (SEQ ID NO: 97) on 10% urea PAGE in an exemplary embodiment of the disclosure.

FIG. 3B shows resolution of 5′ cleavage fragments from screening DNAzyme cleavage of 5′ labelled 32P-RNA nucleocapsid full substrate (SEQ ID NO: 97) on 5% urea PAGE in an exemplary embodiment of the disclosure.

FIG. 4A shows resolution of 5′ cleavage fragments from screening DNAzyme cleavage of 5′ labelled 32P-RNA spike substrates 21655/2240, 22420/23122, 23436/23911, 24108/24665 and 24669/25343 (SEQ ID NO: 100, 101, 102, 103 and 104) on 10% urea PAGE in an exemplary embodiment of the disclosure.

FIG. 4B shows resolution of 5′ cleavage fragments from screening DNAzyme cleavage of 5′ labelled 32P-RNA spike substrates 21655/2240, 22420/23122, 23436/23911, 24108/24665 and 24669/25343 (SEQ ID NO: 100, 101, 102, 103 and 104) on 5% urea PAGE in an exemplary embodiment of the disclosure.

FIG. 5 shows resolution of 5′ cleavage fragments from screening DNAzyme cleavage of 5′ labelled 32P-RNA membrane 26523/27192 (SEQ ID NO: 296) on 5% urea PAGE in an exemplary embodiment of the disclosure.

FIG. 6A shows resolution of 5′ cleavage fragments from screening DNAzyme cleavage of 5′ labelled 32P-RNA RdRp 13469/14676 and 14793/16197 (SEQ ID NO: 98 and 99) on 10% urea PAGE in an exemplary embodiment of the disclosure.

FIG. 6B shows resolution of 5′ cleavage fragments from screening DNAzyme cleavage of 5′ labelled 32P-RNA RdRp 13469/14676 and 14793/16197 (SEQ ID NO: 98 and 99) on 5% urea PAGE in an exemplary embodiment of the disclosure.

FIG. 7A shows resolution of 5′ cleavage fragments from screening DNAzyme cleavage of 5′ labelled 32P-RNA 3CL 10054/10972 (SEQ ID NO: 297) on 10% urea PAGE in an exemplary embodiment of the disclosure.

FIG. 7B shows resolution of 5′ cleavage fragments from screening DNAzyme cleavage of 5′ labelled 32P-RNA 3CL 10054/10972 (SEQ ID NO: 297) on 5% urea PAGE in an exemplary embodiment of the disclosure.

FIG. 8A shows resolution of 5′ cleavage fragments from screening DNAzyme cleavage of 5′ labelled 32P-RNA NSP6 10992/11832 (SEQ ID NO: 298) on 10% urea PAGE in an exemplary embodiment of the disclosure.

FIG. 8B shows resolution of 5′ cleavage fragments from screening DNAzyme cleavage of 5′ labelled 32P-RNA NSP6 10992/11832 (SEQ ID NO: 298) on 5% urea PAGE in an exemplary embodiment of the disclosure.

FIG. 9A shows resolution of 5′ cleavage fragments from screening DNAzyme cleavage of 5′ labelled 32P-RNA NSP8 12098/12679 (sequence number 299) on 10% urea PAGE in an exemplary embodiment of the disclosure.

FIG. 9B shows resolution of 5′ cleavage fragments from screening DNAzyme cleavage of 5′ labelled 32P-RNA NSP8 12098/12679 (SEQ ID NO: 299) on 5% urea PAGE in an exemplary embodiment of the disclosure.

FIG. 10A shows resolution of 5′ cleavage fragments from screening DNAzyme cleavage of 5′ labelled 32P-RNA NSP15 19620/20659 (SEQ ID NO: 300) on 10% urea PAGE in an exemplary embodiment of the disclosure.

FIG. 10B shows resolution of 5′ cleavage fragments from screening DNAzyme cleavage of 5′ labelled 32P-RNA NSP15 19620/20659 (SEQ ID NO: 300) on 5% urea PAGE in an exemplary embodiment of the disclosure.

FIG. 11A shows resolution of 5′ cleavage fragments from screening DNAzyme cleavage of 5′ labelled 32P-RNA methyltransferase 20659/21545 (SEQ ID NO: 301) on 10% urea PAGE in an exemplary embodiment of the disclosure.

FIG. 11B shows resolution of 5′ cleavage fragments from screening DNAzyme cleavage of 5′ labelled 32P-RNA methyltransferase 20659/21545 (SEQ ID NO: 301) on 5% urea PAGE in an exemplary embodiment of the disclosure.

FIG. 12A shows resolution of 5′ cleavage fragments from screening DNAzyme cleavage of 5′ labelled 32P-RNA helicase 16236/18039 (SEQ ID NO: 302) on 10% urea PAGE in an exemplary embodiment of the disclosure.

FIG. 12B shows resolution of 5′ cleavage fragments from screening DNAzyme cleavage of 5′ labelled 32P-RNA helicase 16236/18039 (SEQ ID NO: 302) on 5% urea PAGE in an exemplary embodiment of the disclosure.

FIG. 13A shows resolution of 5′ cleavage fragments from screening DNAzyme cleavage of 5′ labelled 32P-RNA exonuclease 18040/19620 (SEQ ID NO: 303) on 10% urea PAGE in an exemplary embodiment of the disclosure.

FIG. 13B shows resolution of 5′ cleavage fragments from screening DNAzyme cleavage of 5′ labelled 32P-RNA exonuclease 18040/19620 (SEQ ID NO: 303) on 5% urea PAGE in an exemplary embodiment of the disclosure.

FIG. 14A shows resolution of 5′ cleavage fragments from screening DNAzyme cleavage of 5′ labelled 32P-RNA ORF3a 25393/26220 (SEQ ID NO: 304) on 10% urea PAGE in an exemplary embodiment of the disclosure.

FIG. 14B shows resolution of 5′ cleavage fragments from screening DNAzyme cleavage of 5′ labelled 32P-RNA ORF3a 25393/26220 (SEQ ID NO: 304) on 5% urea PAGE in an exemplary embodiment of the disclosure.

FIG. 15A shows resolution of 5′ cleavage fragments from screening DNAzyme cleavage of 5′ labelled 32P-RNA NSP1 266/805 (SEQ ID NO: 305) on 10% urea PAGE in an exemplary embodiment of the disclosure.

FIG. 15B shows resolution of 5′ cleavage fragments from screening DNAzyme cleavage of 5′ labelled 32P-RNA NSP1 266/805 (SEQ ID NO: 305) on 5% urea PAGE in an exemplary embodiment of the disclosure.

FIG. 16A shows resolution of 5′ cleavage fragments from screening DNAzyme cleavage of 5′ labelled 32P-RNA NSP2 805/2719 (SEQ ID NO: 306) on 10% urea PAGE in an exemplary embodiment of the disclosure.

FIG. 16B shows resolution of 5′ cleavage fragments from screening DNAzyme cleavage of 5′ labelled 32P-RNA NSP2 805/2719 (SEQ ID NO: 306) on 5% urea PAGE in an exemplary embodiment of the disclosure.

FIG. 17A shows resolution of 5′ cleavage fragments from screening DNAzyme cleavage of 5′ labelled 32P-RNA NSP3 3027/4791 (SEQ ID NO: 307) on 10% urea PAGE in an exemplary embodiment of the disclosure.

FIG. 17B shows resolution of 5′ cleavage fragments from screening DNAzyme cleavage of 5′ labelled 32P-RNA NSP3 3027/4791 (SEQ ID NO: 307) on 5% urea PAGE in an exemplary embodiment of the disclosure.

FIG. 18A shows the fraction cleavage of screened DNAzymes in nucleocapsid, spike, membrane, RdRp, 3CL, NSP1, ORF3aNSP6, NSP8, NSP15, helicase, exonuclease, NSP2, NSP3 and methyltransferase substrate transcripts in an exemplary embodiment of the disclosure.

FIG. 19 shows a schematic of RNase I activated RCA in an exemplary embodiment of the disclosure.

FIG. 20A shows the digestion of n1 RNA by RNase I in the absence or presence (+Circ RCA1) of complementary circular DNA template in an exemplary embodiment of the disclosure.

FIG. 20B shows the optimization of RNase I concentration for RCA in an exemplary embodiment of the disclosure.

FIG. 21A shows inhibition of n1 RNA digestion by RNase I by adding complementary sequences of various length to the digestion reaction in an exemplary embodiment of the disclosure.

FIG. 21B shows the RCA reaction efficiency of using CDTs with various lengths of complementary regions to the n1 RNA in an exemplary embodiment of the disclosure.

FIG. 22 shows the RNase I activated RCA reaction that occurs specifically in the presence of n1 RNA target oligonucleotide in an exemplary embodiment of the disclosure.

FIG. 23 shows dZ_14172a (SEQ ID NO: 81) cleavage of RdRp 13469/14676 (SEQ ID NO: 98) RNA transcript coupled to RCA using RCA18b (SEQ ID NO: 308) circular template in an exemplary embodiment of the disclosure.

FIG. 24 shows dZ_15165a (SEQ ID NO: 86) cleavage of RdRp 14793/16197 (SEQ ID NO: 99) RNA transcript coupled to RCA using RCA19b (SEQ ID NO: 309) circular template in an exemplary embodiment of the disclosure.

FIG. 25 shows dZ_15202a (SEQ ID NO: 87) cleavage of RdRp 14793/16197 (SEQ ID NO: 99) RNA transcript coupled to RCA using RCA20b (SEQ ID NO: 310) circular template in an exemplary embodiment of the disclosure.

FIG. 26 shows dZ_15282a (SEQ ID NO: 88) cleavage of RdRp 14793/16197 (SEQ ID NO: 99) RNA transcript coupled to RCA using RCA21b (SEQ ID NO: 311) circular template in an exemplary embodiment of the disclosure.

FIG. 27 shows dZ_15439a (SEQ ID NO: 90) cleavage of RdRp 14793/16197 (SEQ ID NO: 99) RNA transcript coupled to RCA using RCA22b (SEQ ID NO: 312) circular template in an exemplary embodiment of the disclosure.

FIG. 28 shows dZ_10491a (SEQ ID NO: 112) cleavage of 3CL 10054/10972 (SEQ ID NO: 297) RNA transcript coupled to RCA using RCA23b (SEQ ID NO: 313) circular template in an exemplary embodiment of the disclosure.

FIG. 29 shows dZ_507a (SEQ ID NO: 215) cleavage of NSP1 266/805 (SEQ ID NO: 305) RNA transcript coupled to RCA using RCA24b (SEQ ID NO: 314) circular template in an exemplary embodiment of the disclosure.

FIG. 30 shows dZ_11697a (SEQ ID NO: 125) cleavage of NSP6 10992/11832 (SEQ ID NO: 298) RNA transcript coupled to RCA using RCA25b (SEQ ID NO: 315) circular template in an exemplary embodiment of the disclosure.

FIG. 31 shows dZ_12202a (SEQ ID NO: 129) cleavage of NSP8 12098/12679 (SEQ ID NO: 299) RNA transcript coupled to RCA using RCA26b (SEQ ID NO: 316) circular template in an exemplary embodiment of the disclosure.

FIG. 32 shows dZ_12290a (SEQ ID NO: 131) cleavage of NSP8 12098/12679 (SEQ ID NO: 299) RNA transcript coupled to RCA using RCA27b (SEQ ID NO: 317) circular template in an exemplary embodiment of the disclosure.

FIG. 33 shows dZ_12350a (SEQ ID NO: 133) cleavage of NSP8 12098/12679 (SEQ ID NO: 299) RNA transcript coupled to RCA using RCA28b (SEQ ID NO: 318) circular template in an exemplary embodiment of the disclosure.

FIG. 34 shows dZ_12495a (SEQ ID NO: 135) cleavage of NSP8 12098/12679 (SEQ ID NO: 299) RNA transcript coupled to RCA using RCA29b (SEQ ID NO: 319) circular template in an exemplary embodiment of the disclosure.

FIG. 35 shows dZ_12618a (SEQ ID NO: 137) cleavage of NSP8 12098/12679 (SEQ ID NO: 299) RNA transcript coupled to RCA using RCA30b (SEQ ID NO: 320) circular template in an exemplary embodiment of the disclosure.

FIG. 36 shows dZ_20134a (SEQ ID NO: 145) cleavage of NSP15 19620/20659 (SEQ ID NO: 300) RNA transcript coupled to RCA using RCA31b (SEQ ID NO: 321) circular template in an exemplary embodiment of the disclosure.

FIG. 37 shows dZ_20412a (SEQ ID NO: 151) cleavage of NSP15 19620/20659 (SEQ ID NO: 300) RNA transcript coupled to RCA using RCA32b (SEQ ID NO: 322) circular template in an exemplary embodiment of the disclosure.

FIG. 38 shows dZ_16583a (SEQ ID NO: 157) cleavage of Helicase 16236/18039 (SEQ ID NO: 302) RNA transcript coupled to RCA using RCA33b (SEQ ID NO: 323) circular template in an exemplary embodiment of the disclosure.

FIG. 39 shows dZ_16727a (SEQ ID NO: 158) cleavage of Helicase 16236/18039 (SEQ ID NO: 302) RNA transcript coupled to RCA using RCA34b (SEQ ID NO: 324) circular template in an exemplary embodiment of the disclosure.

FIG. 40 shows dZ_16912a (SEQ ID NO: 160) cleavage of Helicase 16236/18039 (SEQ ID NO: 302) RNA transcript coupled to RCA using RCA35b (SEQ ID NO: 325) circular template in an exemplary embodiment of the disclosure.

FIG. 41 shows dZ_17522a (SEQ ID NO: 168) cleavage of Helicase 16236/18039 (SEQ ID NO: 302) RNA transcript coupled to RCA using RCA36b (SEQ ID NO: 326) circular template in an exemplary embodiment of the disclosure.

FIG. 42 shows dZ_18470a (SEQ ID NO: 179) cleavage of Exonuclease 18040/19620 (SEQ ID NO: 303) RNA transcript coupled to RCA using RCA37b (SEQ ID NO: 327) circular template in an exemplary embodiment of the disclosure.

FIG. 43 shows dZ_18583a (SEQ ID NO: 181) cleavage of Exonuclease 18040/19620 (SEQ ID NO: 303) RNA transcript coupled to RCA using RCA38b (SEQ ID NO: 328) circular template in an exemplary embodiment of the disclosure.

FIG. 44 shows dZ_18973a (SEQ ID NO: 188) cleavage of Exonuclease 18040/19620 (SEQ ID NO: 303) RNA transcript coupled to RCA using RCA39b (SEQ ID NO: 329) circular template in an exemplary embodiment of the disclosure.

FIG. 45 shows dZ_19033a (SEQ ID NO: 189) cleavage of Exonuclease 18040/19620 (SEQ ID NO: 303) RNA transcript coupled to RCA using RCA40b (SEQ ID NO: 330) circular template in an exemplary embodiment of the disclosure.

FIG. 46 shows dZ_19398a (SEQ ID NO: 193) cleavage of Exonuclease 18040/19620 (SEQ ID NO: 303) RNA transcript coupled to RCA using RCA41b (SEQ ID NO: 331) circular template in an exemplary embodiment of the disclosure.

FIG. 47 shows dZ_1308a (SEQ ID NO: 249) cleavage of NSP2 805/2719 (SEQ ID NO: 306) RNA transcript coupled to RCA using RCA42b (SEQ ID NO: 332) circular template in an exemplary embodiment of the disclosure.

FIG. 48 shows dZ_1940a (SEQ ID NO: 259) cleavage of NSP2 805/2719 (SEQ ID NO: 306) RNA transcript coupled to RCA using RCA43b (SEQ ID NO: 333) circular template in an exemplary embodiment of the disclosure.

FIG. 49 shows dZ_2167a (SEQ ID NO: 262) cleavage of NSP2 805/2719 (SEQ ID NO: 306) RNA transcript coupled to RCA using RCA44b (SEQ ID NO: 334) circular template in an exemplary embodiment of the disclosure.

FIG. 50 shows dZ_2426a (SEQ ID NO: 266) cleavage of NSP2 805/2719 (SEQ ID NO: 306) RNA transcript coupled to RCA using RCA45b (SEQ ID NO: 335) circular template in an exemplary embodiment of the disclosure.

FIG. 51 shows dZ_3072a (SEQ ID NO: 268) cleavage of NSP3 3027/4791 (SEQ ID NO: 307) RNA transcript coupled to RCA using RCA46b (SEQ ID NO: 336) circular template in an exemplary embodiment of the disclosure.

FIG. 52 shows dZ_3706a (SEQ ID NO: 277) cleavage of NSP3 3027/4791 (SEQ ID NO: 307) RNA transcript coupled to RCA using RCA47b (SEQ ID NO: 337) circular template in an exemplary embodiment of the disclosure.

FIG. 53 shows dZ_4076a (SEQ ID NO: 284) cleavage of NSP3 3027/4791 (SEQ ID NO: 307) RNA transcript coupled to RCA using RCA48b (SEQ ID NO: 338) circular template in an exemplary embodiment of the disclosure.

FIG. 54 shows dZ_4118a (SEQ ID NO: 285) cleavage of NSP3 3027/4791 (SEQ ID NO: 307) RNA transcript coupled to RCA using RCA49b (SEQ ID NO: 339) circular template in an exemplary embodiment of the disclosure.

FIG. 55 shows dZ_4148a (SEQ ID NO: 286) cleavage of NSP3 3027/4791 (SEQ ID NO: 307) RNA transcript coupled to RCA using RCA50b (SEQ ID NO: 340) circular template in an exemplary embodiment of the disclosure.

FIG. 56 shows dZ 21086a (SEQ ID NO: 230) cleavage of MethylTransferase 20659/21545 (SEQ ID NO: 301) RNA transcript coupled to RCA using RCA51b (SEQ ID NO: 341) circular template in an exemplary embodiment of the disclosure.

FIG. 57 shows dZ 21338a (SEQ ID NO: 236) cleavage of MethylTransferase 20659/21545 (SEQ ID NO: 301) RNA transcript coupled to RCA using RCA52b (SEQ ID NO: 342) circular template in an exemplary embodiment of the disclosure.

FIG. 58A shows a schematic of toehold-mediated bDNA displacement for the design of a lateral flow device (LFD), where the displacement of bDNA from the tDNA in the presence of the RCAP, leads to the capture of a gold (Au) nanoparticle-conjugated cDNA1 by cDNA2, which is immobilized on the test line of the LFD, in an exemplary embodiment of the disclosure.

FIG. 58B shows a schematic of an electrochemical sensing mechanism for signal detection, based on an electrochemical reporter (E) conjugated to the cDNA1/cDNA2 assembly in an exemplary embodiment of the disclosure.

FIG. 58C shows toehold-mediated bDNA displacement using PAGE in an exemplary embodiment of the disclosure.

FIG. 58D shows a LFD in which the presence of nucleic acid molecules generated from RCA (RCAP) are assessed in a LFD prototype where a test line is clearly visible in the presence of the generated RCAP or control (synthetic RCA monomer) in an exemplary embodiment of the disclosure.

FIG. 59 shows a schematic of bDNA generation by DNAzyme initiated RCA coupled with a nicking enzyme in an exemplary embodiment of the disclosure.

FIG. 60A shows bridging DNA generation by RCA coupled with a nicking enzyme (using denaturing PAGE for data analysis) in an exemplary embodiment of the disclosure.

FIG. 60B shows bridging DNA generation by RCA coupled with a nicking enzyme using real-time fluorescence in an exemplary embodiment of the disclosure.

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. The modifier “about” used in connection with a quantity is inclusive of the stated value and has the meaning dictated by the context (e.g., it includes the degree of error associated with measurement of the particular quantity). When referring to a period such as a year or annually, it includes a range from 9 months to 15 months. All ranges disclosed herein are inclusive of the endpoints and also any intermediate range points, whether explicitly stated or not, and the endpoints are independently combinable with each other.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “and/or” as used herein means that the listed items are present, or used, individually or in combination. In effect, this term means that “at least one of” or “one or more” of the listed items is used or present.

The term “sample” or “test sample” as used herein may refer to any material in which the presence or amount of a target analyte is unknown and can be determined in an assay. The sample may be from any source, for example, any biological (e.g. human or animal samples, including clinical samples), environmental (e.g. water, soil or air) or natural (e.g. plants) source, or from any manufactured or synthetic source (e.g. food or drinks). The sample may be comprised or is suspected of comprising one or more analytes. The sample may be a “biological sample” comprising cellular and non-cellular material, including, but not limited to, tissue samples, saliva, sputum, urine, blood, serum, other bodily fluids and/or secretions. In some embodiments, the sample comprises saliva, sputum, oropharyngeal and/or nasopharyngeal secretions. In some embodiments, the sample comprises saliva.

The term “target”, “analyte” or “target analyte” as used herein may refer to any agent, including, but not limited to, a small inorganic molecule, small organic molecule, metal ion, biomolecule, toxin, biopolymer (such as a nucleic acid, carbohydrate, lipid, peptide, protein), cell, tissue, microorganism and virus, for which one would like to sense or detect. The analyte may be either isolated from a natural source or synthetic. The analyte may be a single compound or a class of compounds, such as a class of compounds that share structural or functional features. The term analyte also includes combinations (e.g. mixtures) of compounds or agents such as, but not limited, to combinatorial libraries and samples from an organism or a natural environment.

The term “treatment or treating” as used herein may refer to an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable.

The term “virus” as used herein may refer to an organism of simple structure, composed of proteins and nucleic acids, and capable of reproducing only within specific living cells, using its metabolism. In some embodiments, the virus is an enveloped virus, a non-enveloped virus, a DNA virus, a single-stranded RNA virus and/or a double-stranded RNA virus. Non-limiting examples of virus include rhinovirus, myxovirus (including influenza virus), paramyxovirus, coronavirus such as SARS-CoV-2, norovirus, rotavirus, herpes simplex virus, pox virus (including variola virus), reovirus, adenovirus, enterovirus, encephalomyocarditis virus, cytomegalovirus, varicella zoster virus, rabies lyssavirus and retrovirus (including HIV).

The term “recognition moiety” as used herein may refer to a moiety comprising a molecule (e.g. compound) such as, but not limited to, a DNAzyme, aptamer, enzyme, antibody, and/or nucleic acid that is able to recognize the presence of an analyte (e.g. bind to the analyte). In some embodiments, the recognition moiety is able to recognize and cleave the analyte. In some embodiments, the recognition moiety comprises a nuclease. In some embodiments, the recognition moiety comprises a DNAzyme.

The term “reporter moiety” as used herein may refer to a moiety comprising a molecule (e.g. compound) for reporting the presence of an analyte. For example, the moiety is used for transducing the presence of an analyte recognized by the recognition moiety to a detectable signal. The reporter moiety may be a detectable label alone, or alternatively, a molecule modified with a detectable label. In some embodiments, the reporter moiety comprises a detectable label that generates a fluorescent, colorimetric, electrochemical, surface plasmon resonance (SPR) or radioactive signal. In some embodiments, the reporter moiety comprises a biopolymer modified with a detectable label. In some embodiments, the reporter moiety comprises a nucleic acid modified with a detectable label.

The term “capture probe” as used herein may refer to a probe that recognizes and binds, directly or indirectly, to a reporter moiety. In some embodiments, the capture probe is immobilized on a solid support. In some embodiments, the capture probe comprises a biopolymer. In some embodiments, the capture probe comprises a nucleic acid sequence that hybridizes to a complementary sequence.

The term “nucleic acid” as used herein may refer to a biopolymer comprising monomers of nucleotides, such as deoxyribonucleic acid (DNA), ribonucleic acid (RNA) and other polynucleotides of modified nucleotides and/or nucleotide derivatives, and may be either double stranded (ds) or single stranded (ss). In some embodiments, modified nucleotides may contain one or more modified bases (e.g. unusual bases such as inosine, and functional modifications to the bases such as amino), modified backbones (e.g. peptide nucleic acid, PNA) and/or other chemically, enzymatically, or metabolically modified forms.

The term “aptamer” as used herein may refer to a short, chemically synthesized nucleic acid molecule or oligonucleotide sequence which can be generated by in vitro selection to fold into specific three-dimensional structures that bind to a specific analyte with dissociation constants, for example, in the pico- to nano-molar range. Aptamers may be single-stranded DNA, and may include RNA, modified nucleotides and/or nucleotide derivatives. Aptamers may also be naturally occurring RNA aptamers termed “riboswitches”. Functional aptamer sequences may also be rationally designed, truncated, conjugated or otherwise modified from original parent (or full length) sequences.

The term “catalytic nucleic acid”, “catalytic DNA”, “deoxyribozyme”, “DNA enzyme” or “DNAzyme” as used herein may refer to a nucleic acid molecule or oligonucleotide sequence that can catalyze or initiate a reaction. DNAzymes may be single-stranded DNA, and may include RNA, modified nucleotides and/or nucleotide derivatives. In some embodiments, the DNAzyme is “RNA-cleaving” and catalyzes the cleavage of a particular substrate, for example a nucleic acid sequence comprising one or more ribonucleotides, at a defined cleavage site. In some embodiments, the substrate is a target nucleic acid in a sample. In some embodiments, the DNAzyme cleaves a single ribonucleotide linkage. In some embodiments, the single ribonucleotide linkage is in a nucleic acid sequence wherein the remaining nucleotides are ribonucleotides. In some embodiments, the single ribonucleotide linkage is in a nucleic acid sequence wherein the remaining nucleotides are deoxyribonucleotides. In some embodiments, the DNAzyme cleaves a nucleic acid sequence at a single ribonucleotide linkage thereby producing a nucleic acid cleavage fragment.

The term “nuclease” as used herein may refer to a protein, such as an enzyme, capable of catalyzing the degradation of a nucleic acid into smaller components by cleaving the phosphodiester bonds between nucleotides of the nucleic acid. Nucleases may be an exonuclease that cleaves a nucleic acid from the ends or an endonuclease that can act on regions in the middle of a nucleic acid. Nucleases may be further subcategorized as a deoxyribonuclease that digests DNA and a ribonuclease that digests RNA.

The term “hybridizes”, “hybridized” or “hybridization” as used herein refers to the sequence specific non-covalent binding interaction with a complementary, or partially complementary, nucleic acid sequence.

The term “rolling circle amplification” or “RCA” as used herein may refer to a unidirectional nucleic acid replication that can rapidly synthesize multiple copies of circular nucleic acid molecules. In some embodiments, rolling circle amplification is an isothermal enzymatic process where a short DNA or RNA primer is amplified to form a long single stranded DNA or RNA using a circular nucleic acid template and an appropriate DNA or RNA polymerase. The product of this process is a concatemer containing ten to thousands of tandem repeats that are complementary to the circular template. A method of RCA comprises annealing a primer to a circular template where the circular template comprises a region complementary to the primer and amplifying the circular template under conditions that allow rolling circle amplification.

Rolling circle amplification conditions are known in the art. For example, rolling circle amplification occurs in the presence of a polymerase that possesses both strand displacement ability and high processivity in the presence of template, primer and nucleotides. In some embodiments, rolling circle amplification conditions comprise temperatures from about 20° C. to about 42° C., or about 22° C. to about 30° C., a reaction time sufficient for the generation of detectable amounts of amplicon and performing the reaction in a buffer. In some embodiments, the rolling circle amplification conditions comprise the presence of Phi29-, Bst-, or Vent exo-DNA polymerase. In some embodiments, the rolling circle amplification conditions comprise the presence of Phi29-DNA polymerase.

The term “sequester” as used herein may refer to a molecule such as nucleic acid that is not available for interaction until it has been released. For example, a first nucleic acid may be in a duplex formation through partial hybridization to a second nucleic acid having an incomplete complementary sequence, and in the presence of a third nucleic acid that has a stronger binding affinity to the second nucleic acid compared to the first nucleic acid, the first nucleic acid is displaced from its interaction with the second nucleic acid, thereby released from its sequestration. As a further example, a bDNA (bridging DNA) may be in a duplex formation through partial hybridization to a tDNA (toehold DNA) such that some amount of the tDNA sequence hangs off the end (i.e. the toehold). In this instance, the bDNA is sequestered. By using the toehold DNA displacement mechanism, the presence of the RCA product (RCAP), the higher complementarity of the tDNA to the RCAP causes the bDNA/tDNA duplex to dissociate, releasing the bDNA from sequestration, making it available for subsequent interactions.

Although methods and materials similar or equivalent to those described herein can be used in the practice or testing of this disclosure, suitable methods and materials are described below.

### II. Recognition Moiety, Biosensors and Biosensor Systems of the Disclosure

The present disclosure discloses a recognition moiety for detecting nucleic acid targets such as SARS-CoV-2 viral RNA.

Accordingly, provided herein is a recognition moiety comprising a catalytic nucleic acid,

wherein the recognition moiety recognizes a target nucleic acid and cleaves the target nucleic acid upon contact to produce a cleavage fragment that acts as a primer for rolling circle amplification (RCA) to generate single-stranded nucleic acid molecules; and

wherein the target nucleic acid is from SARS-CoV-2.

In some embodiments, the catalytic nucleic acid acts as a circular DNA template for performing RCA. In some embodiments, the catalytic nucleic acid comprises a nucleic acid molecule having a sequence as set forth in at least one of SEQ ID NO: 10-15, 17-19, 21, 22, 24, 25, 27, 28, 30, 31, 33, 34, 36, 37, 39, 40, 42, 43, 45, 46, 48, 49, 51, 63-96, and 105-295. In some embodiments, the recognition moiety comprises a nucleic acid molecule having a sequence as set forth in at least one of SEQ ID NO: 16, 20, 23, 26, 29, 32, 41, 72, 76, 80, 81, 86-93, 95, 96, 106-109, 111-117, 119-126, 129, 130, 131, 133, 135, 137, 139, 143, 145, 146, 148, 149, 151, 156-160, 162, 164-168, 176, 179, 181-193, 215, 230, 236, 249, 259, 262, 266, 268, 277, and 284-286. In some embodiments, the recognition moiety comprises a nucleic acid molecule having a sequence as set forth in at least one of SEQ ID NO: 17-19, 21, 22, 66, 80, 81, 91, 92, 96, 109, 123, 112, 114, 130, 139, 145, 151, 160, 179, 182, 203, 215, 230, 236, 249, 259, 262, 266, 268, and 284. In some embodiments, the recognition moiety comprises a nucleic acid molecule having a sequence as set forth in SEQ ID NO: 80. In some embodiments, the recognition moiety comprises a nucleic acid molecule having a sequence as set forth in SEQ ID NO: 92. In some embodiments, the recognition moiety comprises a nucleic acid molecule having a sequence as set forth in SEQ ID NO: 109. In some embodiments, the recognition moiety comprises a nucleic acid molecule having a sequence as set forth in SEQ ID NO: 123. In some embodiments, the recognition moiety comprises a nucleic acid molecule having a sequence as set forth in SEQ ID NO: 130. In some embodiments, the recognition moiety comprises a nucleic acid molecule having a sequence as set forth in SEQ ID NO: 139. In some embodiments, the recognition moiety comprises a nucleic acid molecule having a sequence as set forth in SEQ ID NO: 151. In some embodiments, the recognition moiety comprises a nucleic acid molecule having a sequence as set forth in SEQ ID NO: 179. In some embodiments, the recognition moiety comprises a nucleic acid molecule having a sequence as set forth in SEQ ID NO: 182. In some embodiments, the recognition moiety comprises a nucleic acid molecule having a sequence as set forth in SEQ ID NO: 215. In some embodiments, the recognition moiety comprises a nucleic acid molecule having a sequence as set forth in SEQ ID NO: 249. In some embodiments, the recognition moiety comprises a nucleic acid molecule having a sequence as set forth in SEQ ID NO: 259. In some embodiments, the recognition moiety comprises a nucleic acid molecule having a sequence as set forth in SEQ ID NO: 262. In some embodiments, the recognition moiety comprises a nucleic acid molecule having a sequence as set forth in SEQ ID NO: 266. In some embodiments, the recognition moiety comprises a nucleic acid molecule having a sequence as set forth in SEQ ID NO: 268. In some embodiments, the recognition moiety comprises a nucleic acid molecule having a sequence as set forth in SEQ ID NO: 284. In some embodiments, the recognition moiety comprises a nucleic acid molecule having a sequence as set forth in SEQ ID NO: 112. In some embodiments, the recognition moiety comprises a nucleic acid molecule having a sequence as set forth in SEQ ID NO: 114. In some embodiments, the recognition moiety comprises a nucleic acid molecule having a sequence as set forth in SEQ ID NO: 81. In some embodiments, the recognition moiety comprises a nucleic acid molecule having a sequence as set forth in SEQ ID NO: 91. In some embodiments, the recognition moiety comprises a nucleic acid molecule having a sequence as set forth in SEQ ID NO: 160. In some embodiments, the recognition moiety comprises a nucleic acid molecule having a sequence as set forth in SEQ ID NO: 145. In some embodiments, the recognition moiety comprises a nucleic acid molecule having a sequence as set forth in SEQ ID NO: 230. In some embodiments, the recognition moiety comprises a nucleic acid molecule having a sequence as set forth in SEQ ID NO: 236. In some embodiments, the recognition moiety comprises a nucleic acid molecule having a sequence as set forth in SEQ ID NO: 203. In some embodiments, the recognition moiety comprises a nucleic acid molecule having a sequence as set forth in SEQ ID NO: 96. In some embodiments, the recognition moiety comprises a nucleic acid molecule having a sequence as set forth in SEQ ID NO: 19. In some embodiments, the recognition moiety comprises a nucleic acid molecule having a sequence as set forth in SEQ ID NO: 66. In some embodiments, the recognition moiety comprises a nucleic acid molecule having a sequence as set forth in SEQ ID NO: 22.

In some embodiments, the recognition moiety cleaves a target nucleic acid, wherein the target nucleic acid has a sequence as set forth in at least one of SEQ ID NO: 1-9, 97-104, and 296-307. In some embodiments, the target nucleic acid has a sequence as set forth in at least one of SEQ ID NO: 97-104, 296-300, 302, and 303. In some embodiments, the recognition moiety comprises a nucleic acid molecule having a sequence as set forth in SEQ ID NO: 80, and the target nucleic acid has a sequence as set forth in SEQ ID NO: 98. In some embodiments, the recognition moiety comprises a nucleic acid molecule having a sequence as set forth in SEQ ID NO: 92, and the target nucleic acid has a sequence as set forth in SEQ ID NO: 99. In some embodiments, the recognition moiety comprises a nucleic acid molecule having a sequence as set forth in SEQ ID NO: 109, and the target nucleic acid has a sequence as set forth in SEQ ID NO: 297. In some embodiments, the recognition moiety comprises a nucleic acid molecule having a sequence as set forth in SEQ ID NO: 123, and the target nucleic acid has a sequence as set forth in SEQ ID NO: 298. In some embodiments, the recognition moiety comprises a nucleic acid molecule having a sequence as set forth in SEQ ID NO: 130, and the target nucleic acid has a sequence as set forth in SEQ ID NO: 299. In some embodiments, the recognition moiety comprises a nucleic acid molecule having a sequence as set forth in SEQ ID NO: 139, and the target nucleic acid has a sequence as set forth in SEQ ID NO: 300. In some embodiments, the recognition moiety comprises a nucleic acid molecule having a sequence as set forth in SEQ ID NO: 151, and the target nucleic acid has a sequence as set forth in SEQ ID NO: 300. In some embodiments, the recognition moiety comprises a nucleic acid molecule having a sequence as set forth in SEQ ID NO: 179, and the target nucleic acid has a sequence as set forth in SEQ ID NO: 303. In some embodiments, the recognition moiety comprises a nucleic acid molecule having a sequence as set forth in SEQ ID NO: 182, and the target nucleic acid has a sequence as set forth in SEQ ID NO: 303. In some embodiments, the recognition moiety comprises a nucleic acid molecule having a sequence as set forth in SEQ ID NO: 215, and the target nucleic acid has a sequence as set forth in SEQ ID NO: 305. In some embodiments, the recognition moiety comprises a nucleic acid molecule having a sequence as set forth in SEQ ID NO: 249, and the target nucleic acid has a sequence as set forth in SEQ ID NO: 306. In some embodiments, the recognition moiety comprises a nucleic acid molecule having a sequence as set forth in SEQ ID NO: 259, and the target nucleic acid has a sequence as set forth in SEQ ID NO: 306. In some embodiments, the recognition moiety comprises a nucleic acid molecule having a sequence as set forth in SEQ ID NO: 262, and the target nucleic acid has a sequence as set forth in SEQ ID NO: 306. In some embodiments, the recognition moiety comprises a nucleic acid molecule having a sequence as set forth in SEQ ID NO: 266, and the target nucleic acid has a sequence as set forth in SEQ ID NO: 306. In some embodiments, the recognition moiety comprises a nucleic acid molecule having a sequence as set forth in SEQ ID NO: 268, and the target nucleic acid has a sequence as set forth in SEQ ID NO: 307. In some embodiments, the recognition moiety comprises a nucleic acid molecule having a sequence as set forth in SEQ ID NO: 284, and the target nucleic acid has a sequence as set forth in SEQ ID NO: 307. In some embodiments, the recognition moiety comprises a nucleic acid molecule having a sequence as set forth in SEQ ID NO: 112, and the target nucleic acid has a sequence as set forth in SEQ ID NO: 297. In some embodiments, the recognition moiety comprises a nucleic acid molecule having a sequence as set forth in SEQ ID NO: 114, and the target nucleic acid has a sequence as set forth in SEQ ID NO: 297. In some embodiments, the recognition moiety comprises a nucleic acid molecule having a sequence as set forth in SEQ ID NO: 81, and the target nucleic acid has a sequence as set forth in SEQ ID NO: 98. In some embodiments, the recognition moiety comprises a nucleic acid molecule having a sequence as set forth in SEQ ID NO: 91, and the target nucleic acid has a sequence as set forth in SEQ ID NO: 99. In some embodiments, the recognition moiety comprises a nucleic acid molecule having a sequence as set forth in SEQ ID NO: 160, and the target nucleic acid has a sequence as set forth in SEQ ID NO: 302. In some embodiments, the recognition moiety comprises a nucleic acid molecule having a sequence as set forth in SEQ ID NO: 145, and the target nucleic acid has a sequence as set forth in SEQ ID NO: 300. In some embodiments, the recognition moiety comprises a nucleic acid molecule having a sequence as set forth in SEQ ID NO: 230, and the target nucleic acid has a sequence as set forth in SEQ ID NO: 301. In some embodiments, the recognition moiety comprises a nucleic acid molecule having a sequence as set forth in SEQ ID NO: 236, and the target nucleic acid has a sequence as set forth in SEQ ID NO: 301. In some embodiments, the recognition moiety comprises a nucleic acid molecule having a sequence as set forth in SEQ ID NO: 203, and the target nucleic acid has a sequence as set forth in SEQ ID NO: 304. In some embodiments, the recognition moiety comprises a nucleic acid molecule having a sequence as set forth in SEQ ID NO: 96, and the target nucleic acid has a sequence as set forth in SEQ ID NO: 296. In some embodiments, the recognition moiety comprises a nucleic acid molecule having a sequence as set forth in SEQ ID NO: 19, and the target nucleic acid has a sequence as set forth in SEQ ID NO: 1 or 97. In some embodiments, the recognition moiety comprises a nucleic acid molecule having a sequence as set forth in SEQ ID NO: 66, and the target nucleic acid has a sequence as set forth in SEQ ID NO: 97. In some embodiments, the recognition moiety comprises a nucleic acid molecule having a sequence as set forth in SEQ ID NO: 22, and the target nucleic acid has a sequence as set forth in SEQ ID NO: 2 or 97.

The present disclosure also discloses cleavage-amplification biosensor platform for detecting nucleic acid targets, such as SARS-CoV-2 viral RNA, for use as a simple, non-reverse transcription based POCT.

Accordingly, provided herein is a biosensor for detecting a target nucleic acid comprising a recognition moiety comprising a catalytic nucleic acid, a polynucleotide kinase or phosphatase, and reagents for performing rolling circle amplification (RCA), wherein the recognition moiety cleaves the target nucleic acid to produce a cleavage fragment and the polynucleotide kinase or phosphatase removes cyclic phosphate from the cleavage fragment, producing a dephosphorylated cleavage fragment that acts as a primer for RCA to generate single-stranded nucleic acid molecules. In some embodiments, the biosensor comprises a polynucleotide kinase. In some embodiments, the biosensor comprises a polynucleotide phosphatase.

In some embodiments, the recognition moiety comprises a nuclease. In some embodiments, the recognition moiety comprises a ribonuclease. In some embodiments, the recognition moiety comprises RNase I.

In some embodiments, the reagents for performing RCA comprise a DNA polymerase and deoxyribonucleoside triphosphates. In some embodiments, the reagents for performing RCA comprise a circular DNA template. In some embodiments, the circular DNA template comprises a nucleic acid having a sequence as set forth in any one of SEQ ID NO: 10-15, 17-19, 21, 22, 24, 25, 27, 28, 30, 31, 33, 34, 36, 37, 39, 40, 42, 43, 45, 46, 48, 49, 51, 63-96 and 105-295. In some embodiments, the circular DNA template comprises a nucleic acid having a sequence as set forth in any one of SEQ ID NO: 308-342. In some embodiments, the catalytic nucleic acid is circularized. In some embodiments, the circularized catalytic nucleic acid acts as a circular DNA template for performing RCA. In some embodiments, the target nucleic acid hybridizes to the circular DNA template prior to cleavage by the nuclease.

In some embodiments, the recognition moiety comprises a nucleic acid having a sequence as set forth in SEQ ID NO: 80 and the circular DNA template comprises a nucleic acid having a sequence as set forth in SEQ ID NO: 308. In some embodiments, the recognition moiety comprises a nucleic acid having a sequence as set forth in SEQ ID NO: 81 and the circular DNA template comprises a nucleic acid having a sequence as set forth in SEQ ID NO: 308. In some embodiments, the recognition moiety comprises a nucleic acid having a sequence as set forth in SEQ ID NO: 86 and the circular DNA template comprises a nucleic acid having a sequence as set forth in SEQ ID NO: 309. In some embodiments, the recognition moiety comprises a nucleic acid having a sequence as set forth in SEQ ID NO: 87 and the circular DNA template comprises a nucleic acid having a sequence as set forth in SEQ ID NO: 310. In some embodiments, the recognition moiety comprises a nucleic acid having a sequence as set forth in SEQ ID NO: 88 and the circular DNA template comprises a nucleic acid having a sequence as set forth in SEQ ID NO: 311. In some embodiments, the recognition moiety comprises a nucleic acid having a sequence as set forth in SEQ ID NO: 90 and the circular DNA template comprises a nucleic acid having a sequence as set forth in SEQ ID NO: 312. In some embodiments, the recognition moiety comprises a nucleic acid having a sequence as set forth in SEQ ID NO: 112 and the circular DNA template comprises a nucleic acid having a sequence as set forth in SEQ ID NO: 313. In some embodiments, the recognition moiety comprises a nucleic acid having a sequence as set forth in SEQ ID NO: 215 and the circular DNA template comprises a nucleic acid having a sequence as set forth in SEQ ID NO: 314. In some embodiments, the recognition moiety comprises a nucleic acid having a sequence as set forth in SEQ ID NO: 125 and the circular DNA template comprises a nucleic acid having a sequence as set forth in SEQ ID NO: 315. In some embodiments, the recognition moiety comprises a nucleic acid having a sequence as set forth in SEQ ID NO: 129 and the circular DNA template comprises a nucleic acid having a sequence as set forth in SEQ ID NO: 316. In some embodiments, the recognition moiety comprises a nucleic acid having a sequence as set forth in SEQ ID NO: 131 and the circular DNA template comprises a nucleic acid having a sequence as set forth in SEQ ID NO: 317. In some embodiments, the recognition moiety comprises a nucleic acid having a sequence as set forth in SEQ ID NO: 133 and the circular DNA template comprises a nucleic acid having a sequence as set forth in SEQ ID NO: 318. In some embodiments, the recognition moiety comprises a nucleic acid having a sequence as set forth in SEQ ID NO: 135 and the circular DNA template comprises a nucleic acid having a sequence as set forth in SEQ ID NO: 319. In some embodiments, the recognition moiety comprises a nucleic acid having a sequence as set forth in SEQ ID NO: 137 and the circular DNA template comprises a nucleic acid having a sequence as set forth in SEQ ID NO: 320. In some embodiments, the recognition moiety comprises a nucleic acid having a sequence as set forth in SEQ ID NO: 145 and the circular DNA template comprises a nucleic acid having a sequence as set forth in SEQ ID NO: 321. In some embodiments, the recognition moiety comprises a nucleic acid having a sequence as set forth in SEQ ID NO: 151 and the circular DNA template comprises a nucleic acid having a sequence as set forth in SEQ ID NO: 322. In some embodiments, the recognition moiety comprises a nucleic acid having a sequence as set forth in SEQ ID NO: 157 and the circular DNA template comprises a nucleic acid having a sequence as set forth in SEQ ID NO: 323. In some embodiments, the recognition moiety comprises a nucleic acid having a sequence as set forth in SEQ ID NO: 158 and the circular DNA template comprises a nucleic acid having a sequence as set forth in SEQ ID NO: 324. In some embodiments, the recognition moiety comprises a nucleic acid having a sequence as set forth in SEQ ID NO: 160 and the circular DNA template comprises a nucleic acid having a sequence as set forth in SEQ ID NO: 325. In some embodiments, the recognition moiety comprises a nucleic acid having a sequence as set forth in SEQ ID NO: 168 and the circular DNA template comprises a nucleic acid having a sequence as set forth in SEQ ID NO: 326. In some embodiments, the recognition moiety comprises a nucleic acid having a sequence as set forth in SEQ ID NO: 179 and the circular DNA template comprises a nucleic acid having a sequence as set forth in SEQ ID NO: 327. In some embodiments, the recognition moiety comprises a nucleic acid having a sequence as set forth in SEQ ID NO: 181 and the circular DNA template comprises a nucleic acid having a sequence as set forth in SEQ ID NO: 328. In some embodiments, the recognition moiety comprises a nucleic acid having a sequence as set forth in SEQ ID NO: 188 and the circular DNA template comprises a nucleic acid having a sequence as set forth in SEQ ID NO: 329. In some embodiments, the recognition moiety comprises a nucleic acid having a sequence as set forth in SEQ ID NO: 189 and the circular DNA template comprises a nucleic acid having a sequence as set forth in SEQ ID NO: 330. In some embodiments, the recognition moiety comprises a nucleic acid having a sequence as set forth in SEQ ID NO: 193 and the circular DNA template comprises a nucleic acid having a sequence as set forth in SEQ ID NO: 331. In some embodiments, the recognition moiety comprises a nucleic acid having a sequence as set forth in SEQ ID NO: 249 and the circular DNA template comprises a nucleic acid having a sequence as set forth in SEQ ID NO: 332. In some embodiments, the recognition moiety comprises a nucleic acid having a sequence as set forth in SEQ ID NO: 259 and the circular DNA template comprises a nucleic acid having a sequence as set forth in SEQ ID NO: 333. In some embodiments, the recognition moiety comprises a nucleic acid having a sequence as set forth in SEQ ID NO: 262 and the circular DNA template comprises a nucleic acid having a sequence as set forth in SEQ ID NO: 334. In some embodiments, the recognition moiety comprises a nucleic acid having a sequence as set forth in SEQ ID NO: 266 and the circular DNA template comprises a nucleic acid having a sequence as set forth in SEQ ID NO: 335. In some embodiments, the recognition moiety comprises a nucleic acid having a sequence as set forth in SEQ ID NO: 268 and the circular DNA template comprises a nucleic acid having a sequence as set forth in SEQ ID NO: 336. In some embodiments, the recognition moiety comprises a nucleic acid having a sequence as set forth in SEQ ID NO: 277 and the circular DNA template comprises a nucleic acid having a sequence as set forth in SEQ ID NO: 337. In some embodiments, the recognition moiety comprises a nucleic acid having a sequence as set forth in SEQ ID NO: 284 and the circular DNA template comprises a nucleic acid having a sequence as set forth in SEQ ID NO: 338. In some embodiments, the recognition moiety comprises a nucleic acid having a sequence as set forth in SEQ ID NO: 285 and the circular DNA template comprises a nucleic acid having a sequence as set forth in SEQ ID NO: 339. In some embodiments, the recognition moiety comprises a nucleic acid having a sequence as set forth in SEQ ID NO: 286 and the circular DNA template comprises a nucleic acid having a sequence as set forth in SEQ ID NO: 340. In some embodiments, the recognition moiety comprises a nucleic acid having a sequence as set forth in SEQ ID NO: 230 and the circular DNA template comprises a nucleic acid having a sequence as set forth in SEQ ID NO: 341. In some embodiments, the recognition moiety comprises a nucleic acid having a sequence as set forth in SEQ ID NO: 236 and the circular DNA template comprises a nucleic acid having a sequence as set forth in SEQ ID NO: 342.

In some embodiments, the reagents for performing RCA are comprised in a stabilized composition. In some embodiments, the recognition moiety is comprised in a stabilized composition. In some embodiments, the stabilized composition comprises a stabilizing matrix. In some embodiments, the reagents and/or recognition moiety are encapsulated in a stabilizing matrix. In some embodiments, the stabilizing matrix is a water soluble solid polymeric matrix. In some embodiments, the water soluble solid polymeric matrix is a polysaccharide. In some embodiments, the water soluble solid polymeric matrix comprises pullulan. In some embodiments, the reagents are encapsulated with pullulan. Pullulan is a natural polysaccharide produced by the fungus Aureobasidium pullulans. It readily dissolves in water but resolidifies into films upon drying.

In some embodiments, the biosensor comprises lysis agents. In some embodiments, the lysis agents comprise non-denaturing detergents. In some embodiments, the lysis agents are comprised in a stabilized composition. In some embodiments, the lysis agents are encapsulated in a stabilizing matrix. In some embodiments, the lysis agents are encapsulated with pullulan.

In some embodiments, the biosensor comprises a sample collection device, including, but is not limited to, a vial, a test tube and a microcentrifuge tube. In some embodiments, the biosensor comprises multiple sample collection devices.

In some embodiments, the biosensor comprises a reporter moiety for detection of a signal through RCA. In some embodiments, detection of a signal through RCA indicates the presence of the target in a sample. In some embodiments, the lack of detection of a signal through RCA indicates the absence of the target in a sample. In some embodiments, detection of a signal through RCA indicates presence of single-stranded nucleic acid molecules generated from the RCA reaction. A person skilled in the art would understand that there are numerous ways to detect the presence of single-stranded nucleic acid molecules generated through RCA and includes, without limitation, fluorescent, radioactive, electrochemical, spectroscopic and colorimetric detection and/or quantification. For example, the single-stranded nucleic acid molecules generated through RCA can be labeled radioactively or detected by hybridizing with a complementary nucleic acid molecule, optionally coupled to a detectable label. In some embodiments, the reporter moiety comprises a detectable label that generates a fluorescent, colorimetric, electrochemical, surface plasmon resonance, spectroscopic, or radioactive signal. In some embodiments, the detectable label generates a fluorescent signal. In some embodiments, the detectable label is a fluorescent dye for binding nucleic acids. In some embodiments, the fluorescent dye is SYBR™ Gold, SYBR™ Green or SYBR™ Safe. In some embodiments, the detectable label is an electrochemical label, such as a redox moiety.

In some embodiments, the target nucleic acid comprises RNA. In some embodiments, the target nucleic acid is from a pathogen. In some embodiments, the target nucleic acid is from a virus. In some embodiments, the virus is a coronavirus. In some embodiments, the coronavirus is SARS-CoV-2.

In some embodiments, the recognition moiety comprises nucleic acid molecule having a sequence as set forth in at least one of SEQ ID NO: 10-15, 17-19, 21, 22, 24, 25, 27, 28, 30, 31, 33, 34, 36, 37, 39, 40, 42, 43, 45, 46, 48, 49, 51, 63-96 and 105-295. In some embodiments, the recognition moiety comprises a nucleic acid molecule having a sequence as set forth in at least one of SEQ ID NO: 16, 20, 23, 26, 29, 32, 41, 72, 76, 80, 81, 86-93, 95, 96, 106-109, 111-117, 119-126, 129, 130, 131, 133, 135, 137, 139, 143, 145, 146, 148, 149, 151, 156-160, 162, 164-168, 176, 179, and 181-193. In some embodiments, the recognition moiety comprises a nucleic acid molecule having a sequence as set forth in at least one of SEQ ID NO: 17-19, 21, 22, 66, 80, 81, 91, 92, 96, 109, 123, 112, 114, 130, 139, 145, 151, 160, 179, 182, 203, 215, 230, 236, 249, 259, 262, 266, 268, and 284. In some embodiments, the recognition moiety comprises a nucleic acid molecule having a sequence as set forth in SEQ ID NO: 80. In some embodiments, the target nucleic acid has a sequence as set forth in at least one of SEQ ID NO: 1-9, 97-104, and 296-307. In some embodiments, the target nucleic acid has a sequence as set forth in at least one of SEQ ID NO: 97-104 and 296-300, 302, and 303. In some embodiments, the recognition moiety comprises a nucleic acid molecule having a sequence as set forth in SEQ ID NO: 80, and the target nucleic acid has a sequence as set forth in SEQ ID NO: 98.

In some embodiments, the sample is a biological sample from a subject suspected of having an infection. In some embodiment, the sample is a biological sample from a subject suspected of having a viral infection. In some embodiments, the sample is a biological sample from a subject suspected of having COVID-19. In some embodiments, the biological sample is a sample of saliva, sputum and/or nasopharyngeal secretions, for example, an oropharyngeal and/or nasopharyngeal swab from the subject. In some embodiments, the biological sample is a sample of saliva from the subject.

In some embodiments, the biosensor is for use in screening, diagnostics, and/or health monitoring. In some embodiments, the biosensor is a point-of-care test.

In some embodiments, the biosensor comprises a lateral flow device for detecting the target nucleic acid.

Accordingly, also provided herein is a biosensor system for detecting a target nucleic acid comprising the biosensor as described herein, a second single-stranded oligonucleotide comprising a first domain and a second domain, wherein the single-stranded oligonucleotide is sequestered by a partially complementary oligonucleotide prior to RCA, a reporter moiety complementary to the first domain of the single-stranded oligonucleotide, a capture probe complementary to the second domain of the single-stranded oligonucleotide; and a solid support comprising the capture probe.

In some embodiments, the single-stranded oligonucleotide is partially hybridized to a second single-stranded oligonucleotide complementary to repeating segments of the single-stranded nucleic acid molecules. In some embodiments, the second single-stranded oligonucleotide preferentially hybridizes to the repeating segments of the single-stranded nucleic acid molecules.

In some embodiments, the single-stranded oligonucleotide is generated by cleaving a repeating segment of the single-stranded nucleic acid molecules. In some embodiments, the single-stranded nucleic acid molecules are cleaved by a nicking enzyme. In some embodiments, the nicking enzyme is Nb.BbvCl.

In some embodiments, the solid support comprises a lateral flow test strip. In some embodiments, the lateral flow test strip further comprises a sample pad, a conjugate pad, and an adsorption pad. In some embodiments, the sample pad is a first end of a lateral flow test strip. In some embodiments, the adsorption pad is a second end of a lateral flow test strip. In some embodiments, the reporter moiety is disposed on a conjugate pad on the lateral flow test strip. In some embodiments, the reporter moiety comprises a detectable label. In some embodiments, the detectable label is colorimetric. In some embodiments, the detectable label is a gold nanoparticle. In some embodiments, the capture probe is immobilized on the lateral flow test strip in a visualization area. In some embodiments, the single-stranded oligonucleotide hybridizes to the reporter moiety and the capture probe upon flowing up the lateral flow test strip.

In some embodiments, the solid support comprises an electrode. In some embodiments, the capture probe is immobilized on a sensing region of the electrode. In some embodiments, the single-stranded oligonucleotide hybridizes to the reporter moiety and the capture probe upon disposition on the sensing region of the electrode.

In some embodiments, the biosensor system comprises an aptamer for detecting a non-nucleic acid target in a sample. In some embodiments, detecting a non-nucleic acid target in a sample triggers RCA to generate single-stranded nucleic acid molecules. In some embodiments, the non-nucleic acid target comprises protein. In some embodiments, the non-nucleic acid target is from a pathogen. In some embodiments, the non-nucleic acid target is from a virus. In some embodiments, the virus is a coronavirus. In some embodiments, the coronavirus is SARS-CoV-2. In some embodiments, the aptamer comprises a nucleic acid assembly comprising a primer for RCA. In some embodiments, binding of the aptamer to the non-nucleic acid target releases the primer for RCA to generate single-stranded nucleic acid molecules. In some embodiments, the single-stranded nucleic acid molecules generated through RCA initiated from aptamer binding are detected using the signal detection methods described herein.

In some embodiments, the biosensor system is for use in screening, diagnostics, and/or health monitoring. In some embodiments, the biosensor system is a point-of-care test.

### III. Methods of Detection and Kits of the Disclosure

The present disclosure also provides a method of detecting the presence of a target nucleic acid in a sample comprising contacting the biosensor or biosensor system as described herein with the sample in a solution, allowing for production of an RCA, detecting single-stranded nucleic acid molecules generated from RCA, wherein detection of the single-stranded nucleic acid molecules generated from RCA indicates presence of the target nucleic acid in the sample.

Accordingly, provided is a method for detecting the presence of a target nucleic acid in a sample comprising contacting the sample with a recognition moiety, wherein the recognition moiety cleaves the target nucleic acid to produce a cleavage fragment; removing cyclic phosphate from the cleavage fragment with a polynucleotide kinase or phosphatase; performing rolling circle amplification (RCA) on the cleavage fragment under conditions to generate single-stranded nucleic acid molecules; and detecting the single-stranded nucleic acid molecules generated through RCA wherein detection of the single-stranded nucleic acid molecules generated through RCA indicates presence of the target nucleic acid in the sample. In some embodiments, the method comprises removing cyclic phosphate from the cleavage fragment with a polynucleotide kinase. In some embodiments, the method comprises removing cyclic phosphate from the cleavage fragment with a polynucleotide phosphatase.

In some embodiments, the method comprises contacting the sample with lysis agents prior to contacting the sample with the recognition moiety.

In some embodiments, detection of the single-stranded nucleic acid molecules is indicated by a fluorescent, colorimetric, electrochemical, surface plasmon resonance, spectroscopic, or radioactive signal. In some embodiments, detection of the single-stranded nucleic acid molecules is indicated by a fluorescent signal. In some embodiments, an increase in the fluorescence signal indicates presence of the target nucleic acid in the sample.

In some embodiments, detection of the single-stranded nucleic acid molecules comprises providing a first single-stranded oligonucleotide partially hybridized to a second single-stranded oligonucleotide prior to RCA; preferentially hybridizing the second single-stranded oligonucleotide to repeating segments of the single-stranded nucleic acid molecules produced from the RCA, displacing the first single-stranded oligonucleotide; hybridizing a first domain of the first single-stranded oligonucleotide to a reporter moiety, wherein the reporter moiety is disposed near a first end of lateral flow test strip; flowing the reporter moiety hybridized to the first domain of the first single-stranded oligonucleotide from a first end of the lateral flow test strip towards a second end of the lateral flow test strip; and hybridizing a second domain of the first single-stranded oligonucleotide to a capture probe, wherein the capture probe is immobilized on the lateral flow test strip in a visualization area.

In some embodiments, detection of the single-stranded nucleic acid molecules comprises cleaving a repeating segment of the single-stranded nucleic acid molecules to generate a single-stranded oligonucleotide; hybridizing a first domain of the single-stranded oligonucleotide to a reporter moiety, wherein the reporter moiety is disposed near a first end of lateral flow test strip; flowing the reporter moiety hybridized to the first domain of the single-stranded oligonucleotide from a first end of the lateral flow test strip towards a second end of the lateral flow test strip; and hybridizing a second domain of the single-stranded oligonucleotide to a capture probe, wherein the capture probe is immobilized on the lateral flow test strip in a visualization area.

Provided herein is also a kit for detection of a target nucleic acid in a sample comprising the biosensor or biosensor system as described herein and/or components required for the methods as described herein, and instructions for use of the kit.

In some embodiments, the biosensor, biosensor system, kit and/or method of detection described herein can be used for detecting any suitable analyte, such as, and without being limited thereto, a wide range of small molecule, protein and nucleic acid analytes, including infection-causing pathogens in point-of-care testing for screening, diagnostics and/or health monitoring. Accordingly, provided the use of the biosensor, biosensor system and/or kit as described herein to determine the presence of an analyte in a sample.

In some embodiments, the sample is a biological sample, and the presence of the target nucleic acid in the sample is indicative of, or associated, with a disease, disorder or condition.

In some embodiments, the target nucleic acid comprises RNA. In some embodiments, the target nucleic acid is from a pathogen. In some embodiments, the target nucleic acid is from a virus. Accordingly, provided is a method of detecting a viral infection in a subject comprising testing a sample from the subject for the presence of a target nucleic acid using the biosensor, biosensor system and/or kit described herein, wherein presence of a target nucleic acid indicates that the subject has a viral infection.

In some embodiments, the virus is a coronavirus. In some embodiments, the coronavirus is SARS-CoV-2. In some embodiment, the coronavirus causes COVID-19. In some embodiments, the biosensor, biosensor system and/or kit as disclosed herein can be used in clinical screening and diagnosis of COVID-19. Accordingly, provided herein is a method of detecting COVID-19 in a subject comprising testing a sample from the subject for the presence of SARS-CoV-2 RNA by the methods disclosed herein, wherein the presence of SARS-CoV-2 RNA indicates that the subject has COVID-19. In some embodiments, the method further comprises testing the sample for the presence of SARS-CoV-2 RNA using PCR for validation purposes.

Also provided is a use of the biosensor, biosensor system described herein to determine the presence of a target nucleic acid described herein in a sample.

In accordance with another aspect, there is provided a kit for detection of a target nucleic acid in a sample comprising the biosensor or biosensor system described herein and instructions for use.

In accordance with another aspect, there is provided a kit for detection of a target nucleic acid in a sample, wherein the kit comprises the components required for the methods described herein and instructions for use of the kit.

In accordance with another aspect, there is provided use of the biosensor described herein to determine the presence of an analyte in a sample.

In accordance with another aspect, there is provided use of the biosensor system described herein to determine the presence of an analyte in a sample.

In accordance with another aspect, there is provided use of the kit described herein to determine the presence of an analyte in a sample.

The above disclosure generally describes the present disclosure. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

### EXAMPLES

The following non-limiting examples are illustrative of the present disclosure:

A simple, point-of-care test (POCT) for SARS-CoV-2 that does not require RT and thermophilic DNA polymerases or the expensive equipment used in the current tests has been developed. The tests can be formatted as solution-based fluorescence assays for use with portable fluorescence readers suitable for physician's offices; as color-based lateral flow tests (similar to pregnancy tests) or as electrochemical sensors (similar to glucose meters) to allow for self-testing by untrained users. Such tests would be suitable to be performed by home users and could improve the rate of testing for priority populations such as older adults, residents of long-term care homes, and those in remote locations who do not have access to centralized testing facilities.

### Example 1. DNAzyme-Based Detection of Viral RNA

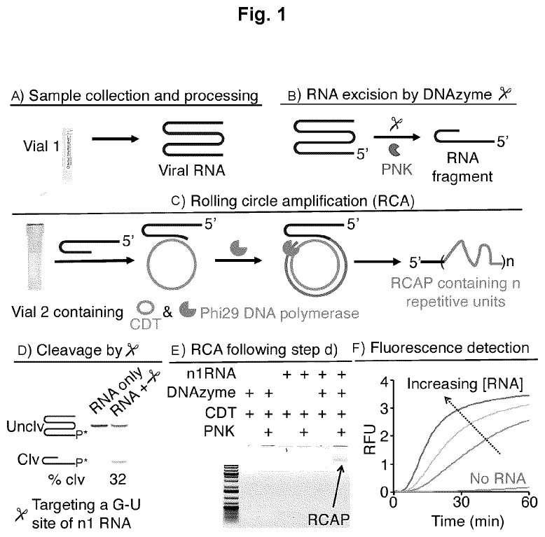

Key RNA sequences of the SARS-CoV-2 virus have been validated and used by, for example, government health institutes (e.g. China's CDC, Germany's Charite, Japan's National Institute of Infectious Diseases and USA's CDC) for diagnosing COVID-19 using RT-PCR assays. Therefore, to develop a simple and rapid test that avoids the need for the common reagents used for RT-PCR based tests and minimize false positives and negatives, DNAzymes sequences (see all oligonucleotide sequences in Table 1) were designed to cleave the SARS-CoV-2 viral RNA genome at positions within or near these key RNA genomic sequence regions (such as the RNA of the E, 5-UTR and N genes; Table 2). Further, DNAzymes were designed based on RNA secondary structure prediction of viral genes, targeting weakly structured regions (denoted as “dZ” series in Tables 1 and 2). A schematic overview of the DNAzyme-based POCT for detecting SARS-CoV-2 viral RNA is depicted in FIG. 1. Briefly, a swab can be used to collect oropharyngeal or nasopharyngeal samples (of saliva, sputum and/or other mucosal secretions that may contain the virus if a person is infected). The swab can be added to a container, such as a small vial (denoted “Vial 1”), containing non-denaturing detergent based viral lysis agents to release viral RNA (and proteins) in a small volume (<1 mL; FIG. 1A).^{[4]} A 10-23 RNA-cleaving DNAzyme,^{[5,6]} is designed to specifically cut the viral RNA at specific target sites, which were selected based on the presence of a purine-pyrimidine dinucleotide junction suitable for cleavage by the 10-23 DNAzyme. High sensitivity is achieved by linking the RNA recognition and catalytic event to an equipment-free room temperature isothermal DNA amplification method known as “rolling circle amplification” (RCA).^{[7,8]} To facilitate RCA, PNK is used to remove the 2′,3′-cyclic phosphate at the end of cleavage product (FIG. 1B).^{[9]} After 10 min, this sample is added directly to “Vial 2”, containing reagents for RCA (including Phi29DP, a CDT and dNTPs), with no need for an RNA extraction step. As shown in FIG. 1C, RCA proceeds by Phi29DP using the cleaved viral RNA as a primer to perform round-by-round extension around the CDT. Importantly, this method can operate at room temperature, avoiding the need for equipment for temperature control. Previous work using an exponentially amplifying version of RCA, known as hyperbranched RCA (HRCA), for detecting microRNAs, has shown this method is extremely sensitive,^{[8]} which should permit robust detection of ˜100 virus copies in about 30 min, which is significantly lower than the reported viral load (10³-10⁷ copies/mL) in saliva or sputum.^{[10]}

The lysis and RCA reagents in Vial 1 and Vial 2, respectively, can be formed as a dry tablet formulated with pullulan,^{[11,12]} which stabilizes enzymes and other molecules. Addition of samples to each vial, causes rehydration of the tablet allowing the entrapped enzymes and other molecules to function without having been degraded while in the dry form.

Using the dry tablet format to stabilize reaction reagents, the procedure may also be further simplified in a single vial format using, for example, tablets of different sizes or compositions to rehydrate at different rates.

### Methods

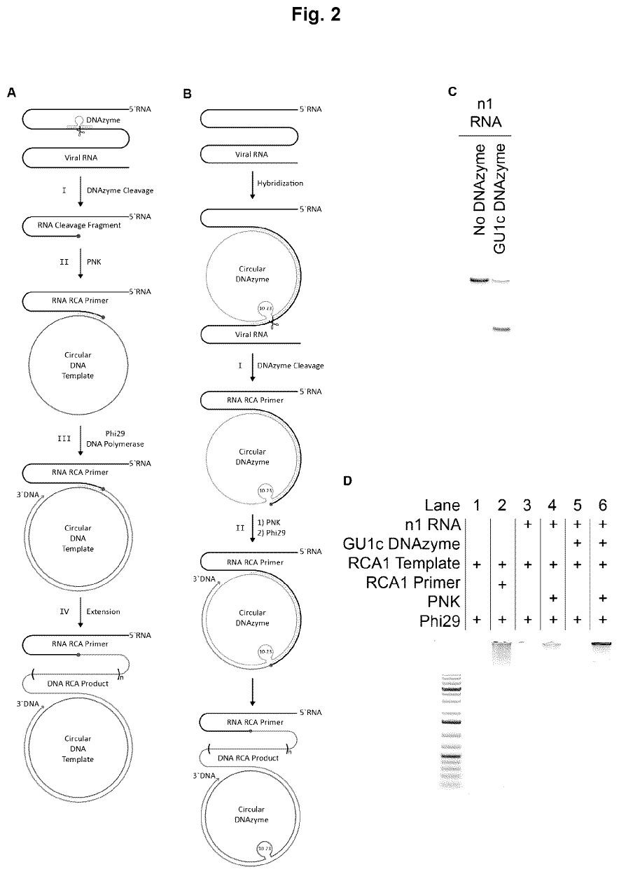

Conceptual design and preparation of oligonucleotides: RNA substrates (SEQ ID NO: 1-9, 97-104 and 296-307) were designed to provide test substrates for DNAzyme analysis based on the cleavage targets of DNAzymes (Table 3). For example, RNA substrates were generated by subcloning 105 bp fragments from a vector containing a SARS-CoV-2 nucleocapsid (N) gene followed by RNA transcription with T7 RNA polymerase (Invitrogen T7 RNA Polymerase). Transcripts were dephosphorylated by alkaline phosphatase (Thermo FastAP), 5′ radiolabeled with γ32p-ATP by PNK (Thermo PNK) reaction and purified by denaturing urea PAGE. The 10-23 DNAzyme sequences were designed with binding arms targeting a specific site within the SARS-CoV-2 viral RNA genome, such that site-directed DNAzyme cleavage of the RNA generates an RNA primer for RCA as depicted in the schematic of FIG. 2A. In I) an RNA substrate is specifically bound by a 10-23 DNAzyme and cleaved, II) the 3′ RNA cleavage fragment is activated for priming by removal of 3′ cyclic phosphate using PNK, III) Phi29DP catalyzes the polymerization of DNA from the 3′ RNA terminal templated by a complementary circular DNA (RCA1), IV) Phi29DP continues polymerization around the circular DNA template generating long repetitive sequence DNA. An alternative scheme is depicted in FIG. 2B using a DNAzyme embedded within a circular RCA template such that the DNAzyme not only cleaves the RNA sequence but is involved in the RCA reaction.

10-23 DNAzyme sequences designed with binding arms targeting a specific site within the SARS-CoV-2 N1 nucleocapsid gene (n1 RNA), such as GU1c, were made first for initial testing (Table 3). DNA sequences were ordered from IDT and purined by denaturing PAGE.

DNAzyme cleavage screening: 10-23 DNAzyme sequences were designed with binding arms targeting a specific site within the SARS-Cov-2 viral gene transcripts based on secondary structure prediction performed using RNAfold WebServer (http://ma.tbi.univie.ac.at/cgi-bin/RNAWebSuite/RNAfold.cgi). Cleavage reactions were performed with 500 nM 10-23 DNAzyme and <50 nM 32P-RNA in reaction buffer (50 nM HEPES pH 7.4, 100 mM NaCl and 10 mM MgCl₂). Reactions were initiated by addition of reaction buffer followed by incubation at 23° C. for 10 minutes. Reactions were quenched by addition of EDTA to 30 mM. Cleavage fragments were analyzed by resolution on 10% and/or 5% urea PAGE.

DNAzyme mediated cleavage of N1 nucleocapsid RNA: A reaction containing 100 nM 5′³²P radiolabeled RNA (n1 RNA) and 500 nM n1GU1c DNAzyme was annealed by heating at 90° C. for 2 minutes and cooling at 23° C. for 5 minutes. The cleavage reaction was initiated by addition of Buffer 1 to 1× (50 nM HEPES pH 7.4, 10 mM MgCl2, 100 nM NaCl) and IOU PNK (Thermo Fisher Scientific) and incubated at 23° C. for 10 minutes or 1 hour for FIG. 1 and FIG. 2, respectively, final volume 10p. Reactions were stopped by addition of EDTA to 30 mM final concentration. Reaction products were resolved on 10% TBE 7 M urea PAGE. RNA cleavage products were visualized by storage phosphor screen and imaged on a Typhoon Biomolecular Imaging system. Band densitometry was performed with ImageJ and calculation of cleavage fraction was done with Microsoft Excel.

Analysis of RCA product from DNAzyme cleavage reactions: For FIG. 1, cleavage reactions were diluted 1:3 by supplementation with 33 nM RCA1 CDT, 1× buffer Phi29DP, 333 μM dNTP and IOU Phi29DP (Thermo Fisher Scientific), final volume 30 μl. Reactions were incubated at 30° C. for 10 minutes. For FIG. 2, replicate cleavage reactions from panel c) subjected to 10 U PNK (Thermo Fisher Scientific) or received no PNK as indicated and incubated at 37° C. for 30 minutes. Reactions were then diluted 1:3 by supplementation with 33 nM RCA1 CDT, 1×Phi29DP buffer, 333 μM dNTP, 33 nM RCA1 primer control as indicated and 10 U Phi29DP (Thermo Fisher Scientific), final volume 30 μl. Reactions were incubated at 30° C. for 30 minutes. Reactions products were run on 1% TAE agarose cast with 1×SYBR™ Safe gel stain (Invitrogen). 2 μl Generuler 1 KB+ was run as size reference (Thermo Fisher Scientific). Gel was visualized by fluorescence scan using a Typhoon Biomolecular Imaging system.

Fluorescence detection of viral RNA cleavage fragments: DNAzyme cleavage reactions were performed as described above, with a range of n1 RNA concentrations ranging from 0-30 nM. Cleavage reactions were diluted 1:3 by supplementation with 33 nM RCA1 CDT, 1×Phi29DP buffer, 1×SYBR™ Gold nucleic acid stain (Invitrogen), 333 μM dNTP and 10 U Phi29DP (Thermo Fisher Scientific), final volume 30 μl. Reactions were incubated at 30° C. in a BioRad CFX-96 realtime thermal cycler and fluorescence measurement collected at one minute intervals for one hour. Raw fluorescence measurements were normalized and plotted using Microsoft Excel.

### Results

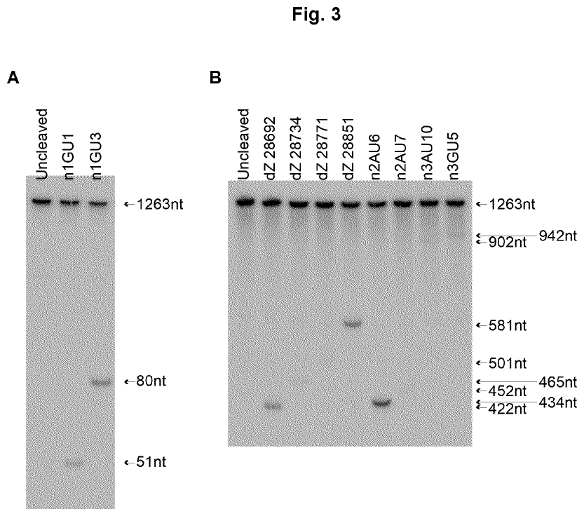

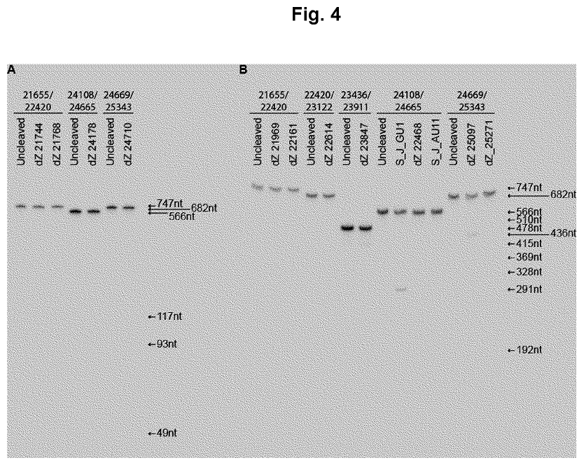

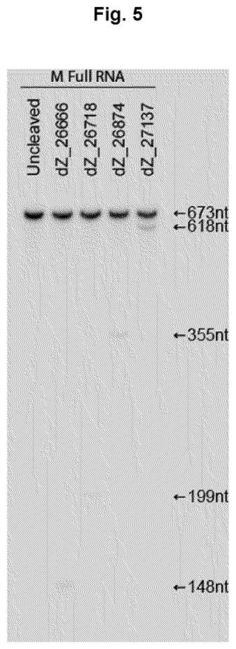

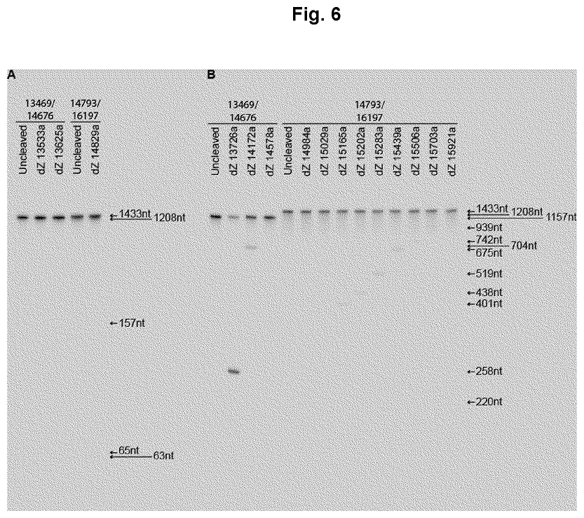

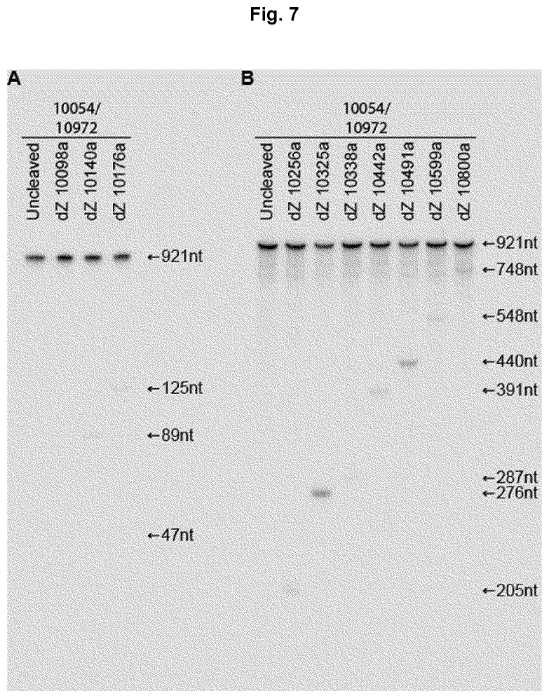

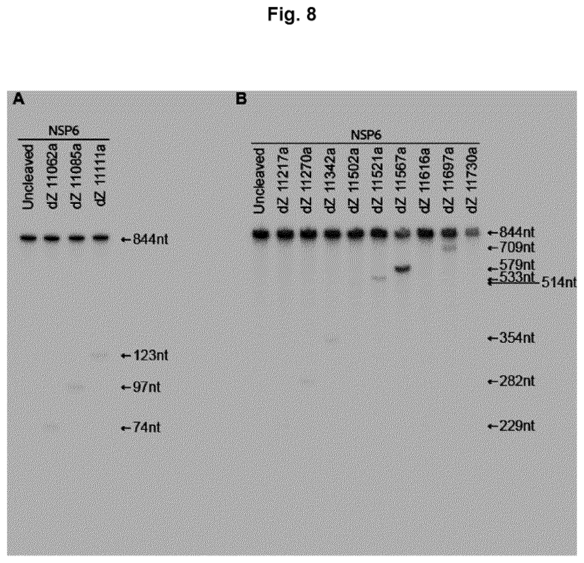

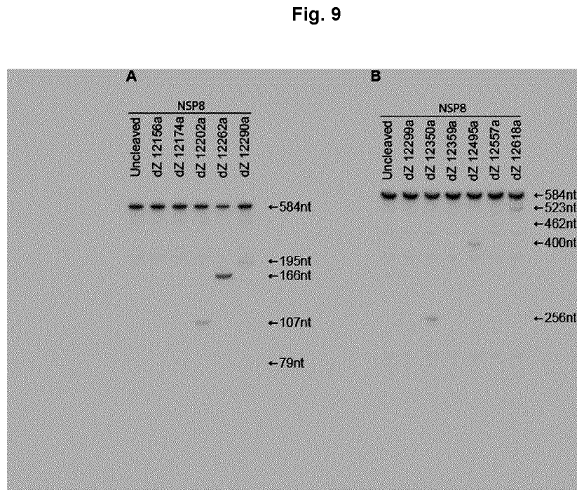

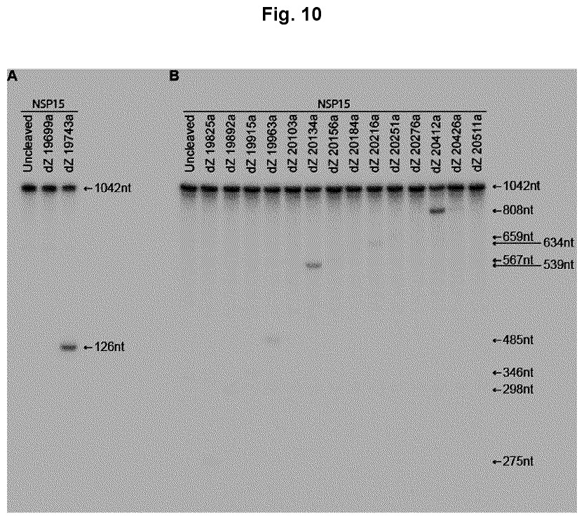

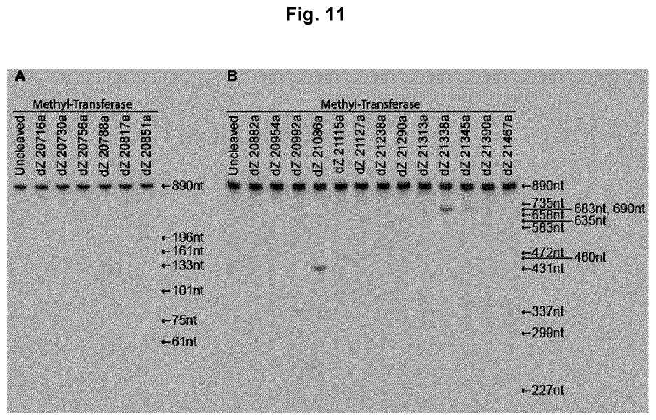

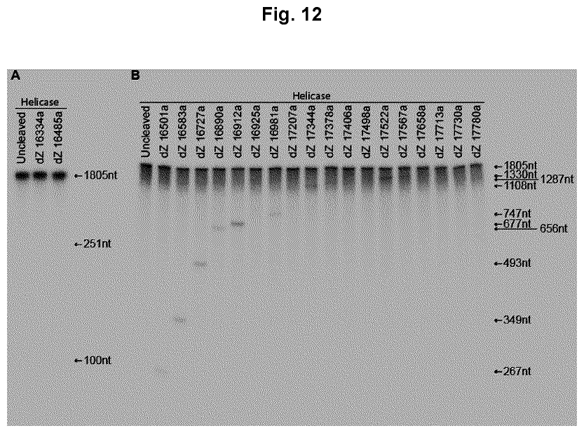

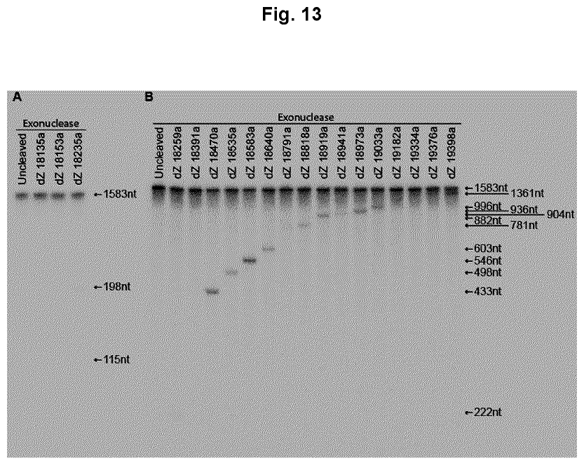

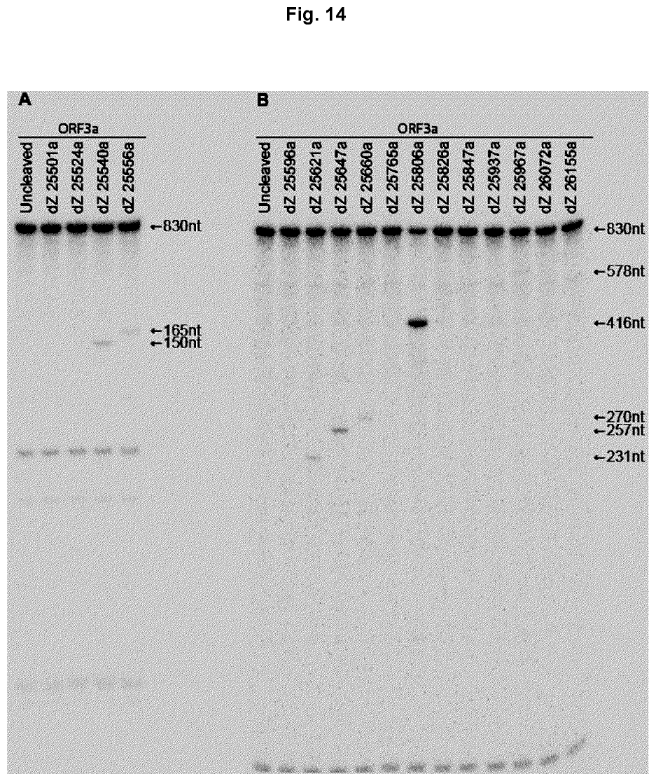

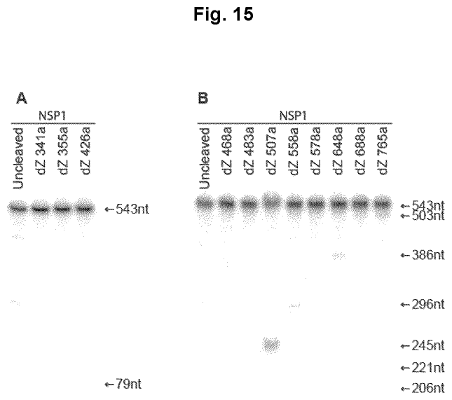

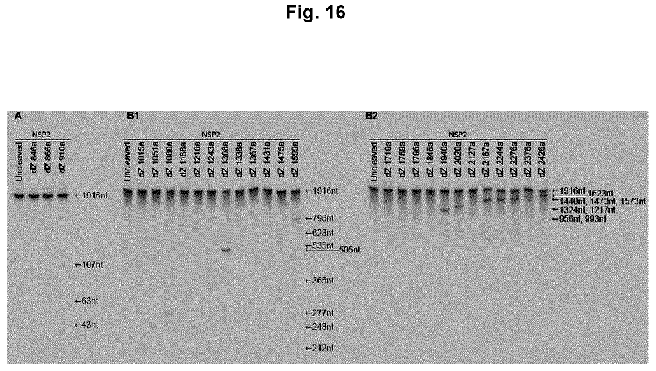

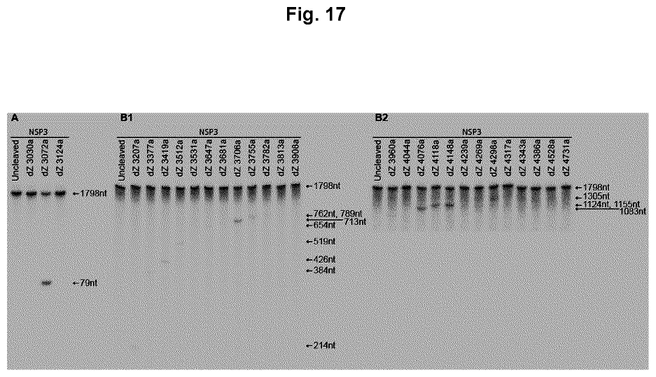

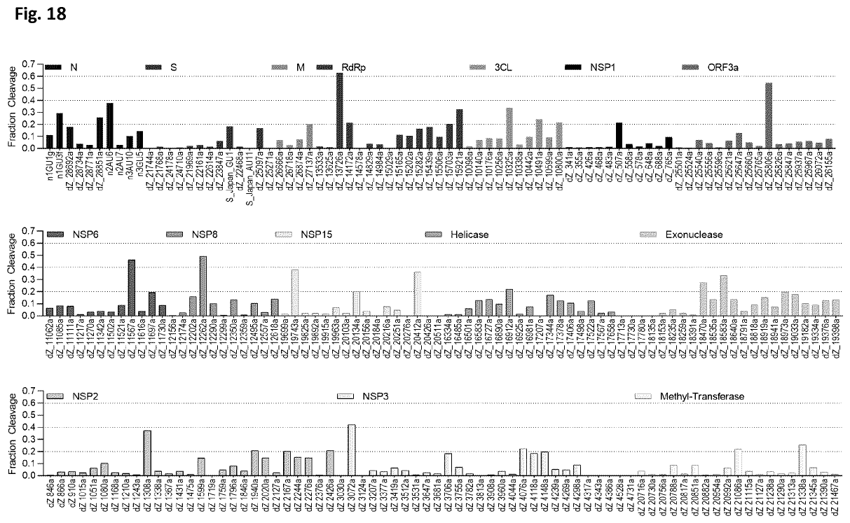

Cleavage by DNAzyme sequences designed for targeting the full nucleocapsid (FIG. 3), spike 21655/2240, 22420/23122, 23436/23911, 24108/24665 and 24669/25343 (FIG. 4), membrane 26523/27192 (FIG. 5), RdRp 13469/14676 and 14793/16197 (FIG. 6), 3CL 10054/10972 (FIG. 7), NSP6 10992/11832 (FIG. 8), NSP8 12098/12679 (FIG. 9), NSP15 19620/20659 (FIG. 10), methyltransferase 20659/21545 (FIG. 11), helicase 16236/18039 (FIG. 12), exonuclease 18040/19620 (FIG. 13), ORF3a 25393/26220 (FIG. 14), NSP1 266/805 (FIG. 15), NSP2 805/2719 (FIG. 16) and NSP3 3027/4791 (FIG. 17) substrate transcripts were assessed. Fraction cleavage of screened DNAzymes is summarized in FIG. 18.

The GU1c DNAzyme is capable of efficiently cleaving N1 nucleocapsid RNA at a specific G-U junction (FIG. 1D and FIG. 2C; the RNA has a radioactive 5′-phosphate, P*). In 10 minutes, the DNAzyme cleaved ˜30% of the total RNA (“Clv”: 5′-cleavage fragment, which runs faster than uncleaved RNA, “Unclv”, on polyacrylamide gel).

This reaction mixture was then used to conduct RCA in Vial 2, as the RNA cleavage fragments generated by DNAzyme cleavage serve as primers to complementary circular templates for RCA (Table 4), generate a large amount of output DNA (product of the RCA reaction) for detection.

As shown in FIG. 1E and FIG. 2D, significant RCAP is generated by DNAzyme cleaved RNA. The RCAP can be detected visually on a gel (as well as imaged and quantified) by labeling the RCAP with fluorescent DNA-binding dyes, such as SYBR™ Safe gel stain. Directly monitoring the RCA reaction and generation of RCAP by fluorescence (FIG. 1F) allows for the development of lab-based tests using assay formats amenable to multiplexing and high-throughput screening such as fluorescence-based microtiter well plate readers.

### Example 2. RNase I Activated RCA

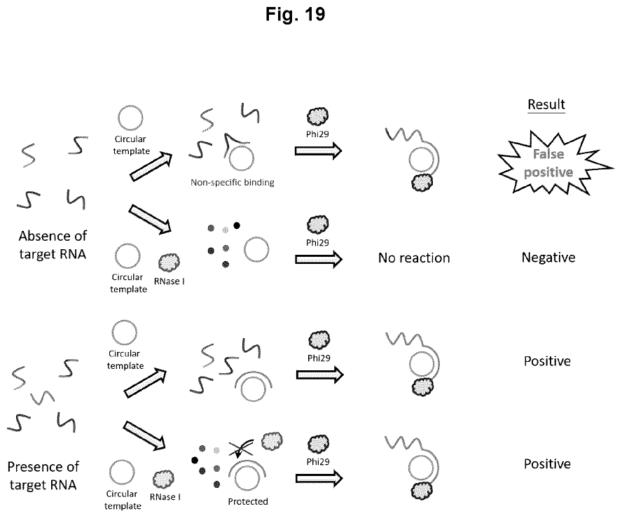

As shown in FIG. 19, RNase I was used to specifically cleave target RNA and activate RCA. In the absence of target RNA, when the sample was incubated with circular template, non specific binding of RNA fragments to the circular template could occur, which could initiate RCA by Phi29DP, and lead to a false positive. To mitigate this issue, RNase I was incubated with the sample and CDT. This led to the digestion of the non-specific RNA fragments, and no RCA product was produced. In the presence of the target RNA the RNase I still functioned to decrease background amplification by eliminating competitive non-specific RNA fragments. In the presence of the target RNA and circular template, the target RNA bound to the CDT and initiated RCA, to yield a positive test result. When RNase I was added, it degraded competing and non-competing non-specific RNA fragments allowing for the efficient and specific amplification of the target RNA by Phi29DP to produce an RCA product, leading to a positive test.

### Methods

Digestion of n1 RNA by RNase I: The reaction was assembled by combining 10 nM ³²P labelled n1 RNA (1 μL), 0.1 μM RCA1 CDT (1 μL), Phi29DP reaction buffer (1 μL), and ddH₂O to a total of 9 μL. RNase I (1 μL) was then added and mixed by pipette. The reaction was incubated at 30° C. for 10 minutes. To analyze the reaction, the reaction product (10 μL) was run on a 10% urea denaturing PAGE at 35 W for 20 min.

RNase I concentration optimization: the reaction was assembled by combining 10 nM ³²P labelled n1 RNA (1 μL), 0.1 μM RCA1 CDT (1 μL), Phi29DP reaction buffer (1 μL), and ddH₂O up to 9 μL. Subsequently RNase I (1 μL) was added and mixed by pipette. The reaction was incubated at 30° C. for 10 minutes, then the reaction product (10 μL) was analyzed using 10% urea denaturing PAGE at 35 W for 20 min.

Optimization of circular templates for n1 RNA complementarity and RNase I activated RCA: circular sequences with various complementarity that ranged from 16 nt to 35 nt to the n1 RNA target were designed and are shown in Table 3. To examine which oligonucleotide showed the best protective effect, reactions were assembled by combining 10 nM ³²P labelled n1 RNA (1 μL), 0.1 μM CDT (1 μL), Phi29DP reaction buffer (1 μL), and ddH₂O to 9 μL. Subsequently, 0.005 U RNase I (1 μL) was added and mixed by pipette. The reactions were incubated at 30° C. for 10 minutes, then the reaction product (10 μL) was run on a 10% urea denaturing PAGE at 35 W for 20 min.

RCA reaction with extended circular template: the reaction was assembled by combining 0.1 μM CDT (1 μL), 0.005 U RNase I (1 μL), 10 U Phi29 (1 μL), 10 mM dNTP (1 μL), Phi29DP reaction buffer (1 μL), and ddH₂O up to 9 μL. Subsequently, n1 RNA (1 μL) was added and mixed by pipette. The reactions were incubated at room temperature for 15 minutes then the reaction product (10 μL) was run on a 0.6% agarose gel stained with SYBR™ Safe at 100 W for 60 min.

RNase I activated RCA in the presence of n1 RNA: the reaction was prepared by adding 0.1 μM CDT (1 μL), 0.05 U RNase I (1 μL), 10 U Phi29DP (1 μL), 10 mM dNTP (1 μL), Phi29 reaction buffer (1 μL), and ddH₂O to 9 μL. Subsequently, n1 RNA (1 μL) was added and the reaction was mixed by pipette. The reactions were incubated at room temperature for 15 minutes. Half of the reaction product was mixed with 50 nM cDNA and BamHI for single unit digestion. Finally, the reactions were analyzed by 0.6% agarose gel stained with SYBR™ Safe at 100 W for 60 min.

### Results

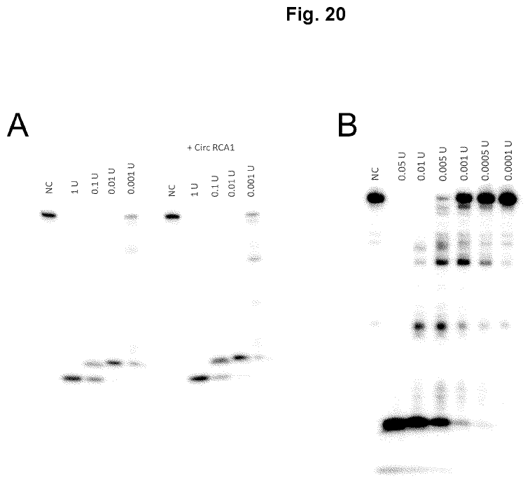

To begin to examine the RNase I activated RCA method, first the digestion of n1 RNA by RNase I was investigated. FIG. 20A show that the digestion of ³²P-labelled n1 RNA by RNase I was achieved in the absence of the CDT, and decreased in the presence of a CDT (+Circ RCA1). This trend was most evident at the RNase I concentration of 0.001 U, where additional bands are evident in the presence of the +Circ RCA1 compared to in its absence. This indicates that the CDT RCA1 prevented the digestion of n1 RNA by RNase I, and that n1 RNA can be used as primer of RCA reaction. The negative controls (NC) in the panels were ³²P-labelled n1 RNA and RCA buffer only, without the CDT or RNase I.

The concentration of RNase I was then optimized for best performance of activated RCA reaction (FIG. 20B). At the concentration equal and lower than 0.0005 U, only minor fraction of n1 RNA was digested and the fragments of digested n1 RNA were barely observed. On the other hand, the n1 RNA is completely digested with the RNase I concentration higher than 0.05 U and almost no fragments were observed. Therefore, using appropriate RNase I concentration is critical to provide as many n1 RNA fragments for the RCA reaction as possible. The negative control (NC) in this figure contained ³²P-labelled n1 RNA, CDT RCA1 and RCA buffer, without RNase I.

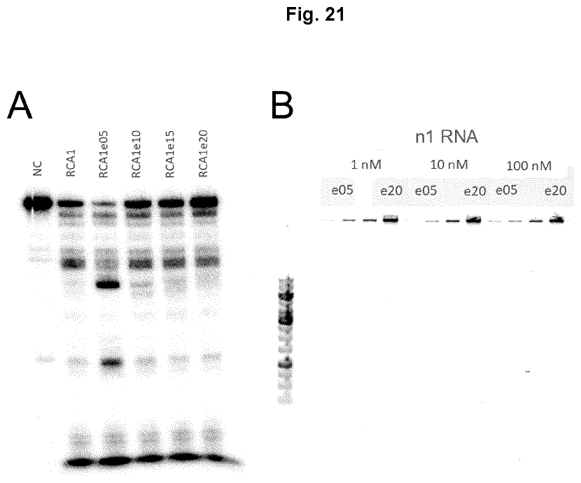

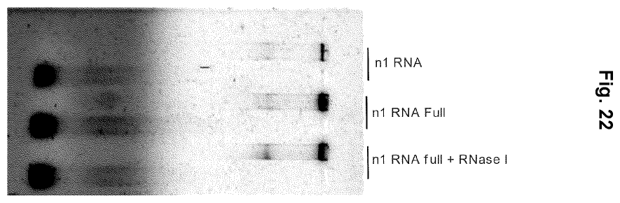

The n1 RNA digestion by RNase I is inhibited by adding complementary sequence (FIG. 21A). Herein, four additional CDTs with extended regions for hybridization were examined. The hybridized base pairs with n1 RNA were 16 nt (RCA1), 21 nt (RCA1e05), 26 nt (RCA1e10), 31 nt (RCA1e15) and 36 nt (RCA1e20), in length respectively. The negative control (NC) in this experiment contained the ³²P-labelled n1 RNA, CDT RCA1, and RCA buffer. No RNase I was included. This assay revealed that the more base pairs hybridized between the two oligonucleotides, the better the protection from RNase I digestion. However, a higher digestion ratio of RCA1e05 was observed at lane 3 in FIG. 20A. This unusual trend is due to the intramolecular interaction of RCA1e05, the secondary structure of RCA1e05 made a lesser fraction of n1 RCA hybridize to the CDT and be protected from RNase I digestion. This phenomenon was further verified by the estimated Tm values of RCA1e05 (69.4° C.) and RCA1 (71.7° C.).

As shown in FIG. 21B, the RNase I activated RCA products were significantly increased with extended hybridization region between n1 RNA and the CDT. These results were indicative that the stronger binding between n1 RNA and the CDT, the more products produced by the RNase I activated RCA reaction.

Finally, the full length of n1 RNA was examined as a primer for RNase I activated RCA assay (FIG. 22). In this experiment, each set of reactions was treated with complementary DNA and endonuclease BamHI after the RCA reaction to verify that the bands observed on the image were RCA products. In this experiment the n1 RNA is a 105 nt fragment of the n1 RNA full, which is 1263 nt. As shown in FIG. 22, sets 2 (n1 RNA full, lanes 4 and 5) and 3 (n1 RNA full +RNase I, lanes 6 and 7) indicate the full length of n1 RNA is able to activate the RCA reaction correctly. Moreover, the RNase I digestion initiates more efficient RCA reactions as shown by fewer low molecular weight bands in set 3 than set 2 or set 1 (the control n1 RNA). Importantly, bands from each of the 3 sets were vanished after treating with BamHI (lanes 3, 5, and 7) leading to a large number of short fragments which appeared at lower molecular weight regions on the gel. These results indicated that the higher molecular weight bands observed in lanes 2, 4, and 6, were RCA products that were cleaved into mono units by endonuclease (lanes 3, 5 and 7).

### Example 3. RCA Activated by DNAzyme Cleavage in Saliva Matrix

Fluorescence intensity (relative fluorescence units; RFU) generated from coupled DNAzyme-RCA reactions was measured using DNAzyme sequences for targeting RNA transcripts of RdRp, 3CL, NSP1, NSP2, NSP3, NSP6, NSP8, NSP15, helicase, exonuclease and methyltransferase.

### Methods

Using human pooled saliva (Innovative Research) treated with 2.5 mg/ml Proteinase K (Thermo Scientific) and heated at 90° C. for 10 minutes. Select 10-23 DNAzyme sequences were used to cleave complementary in vitro transcribed RNA substrates (50 nM DNAzyme:10 nM RNA transcript) in reactions containing 50% v/v treated human pooled saliva. RNA cleavage reactions were initiated with reaction buffer (previously described) and incubated at 23° C. for 1 hour. Cleavage reactions are diluted 1:1 with RCA reagents (10 nM circular RCA template, 250 μM dNTP, 1× SybrGold, 0.25 U/μl PNK, 0.25 U/μl phi29 DNA polymerase and 1×phi29 reaction buffer) and incubated at 23° C. for 4 hours using a Biorad CFX-96 realtime thermal cycler while monitoring fluorescence.

### Results

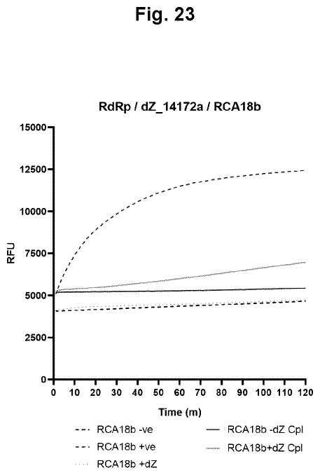

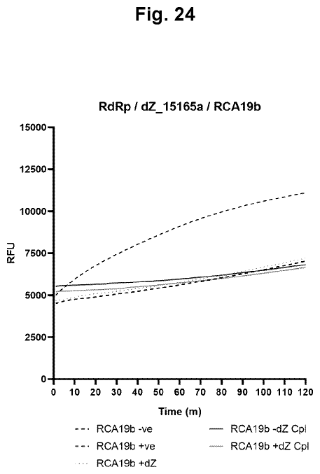

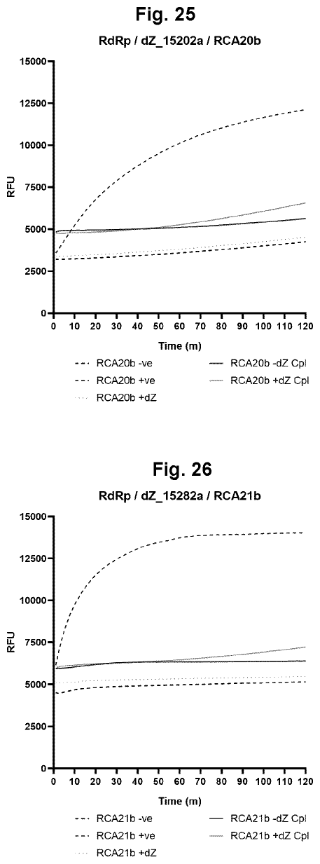

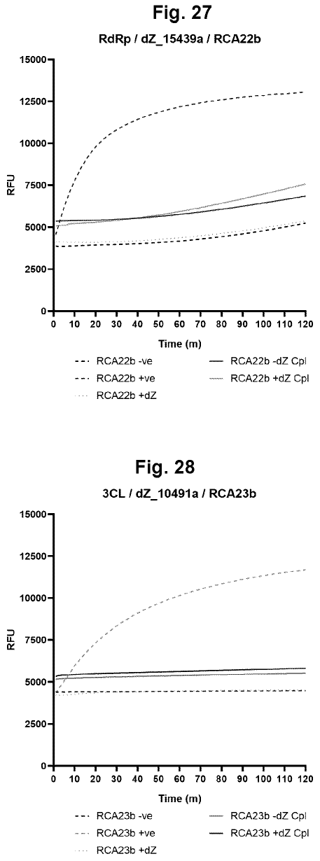

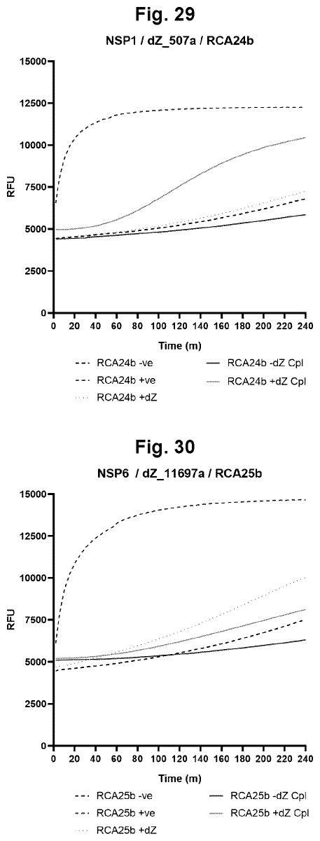

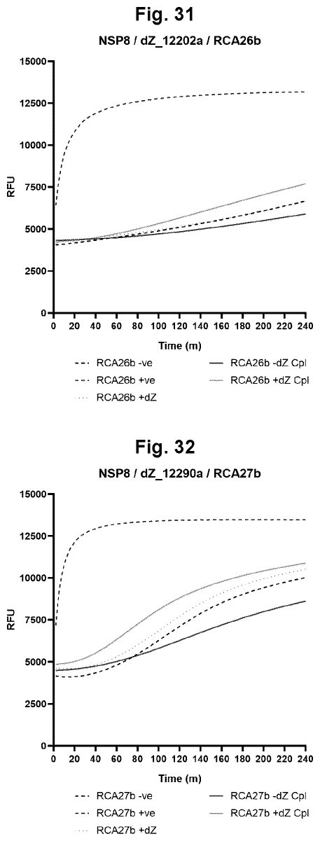

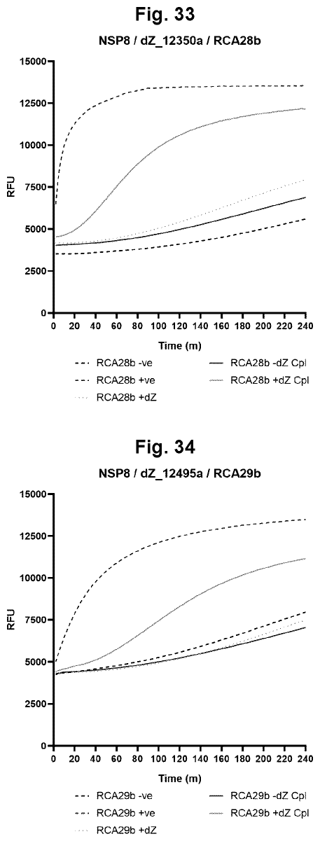

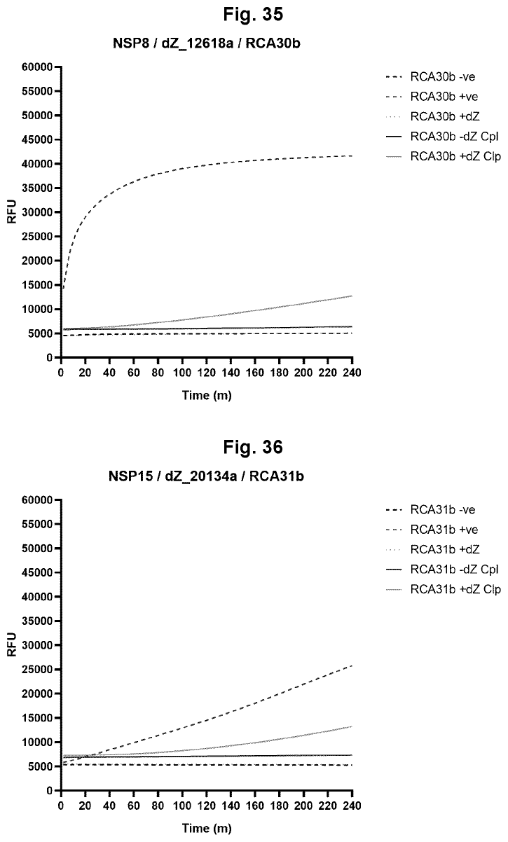

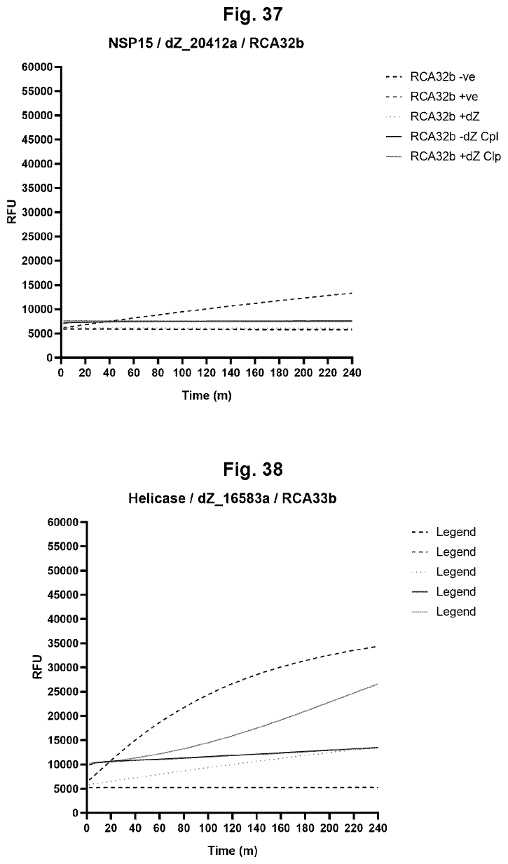

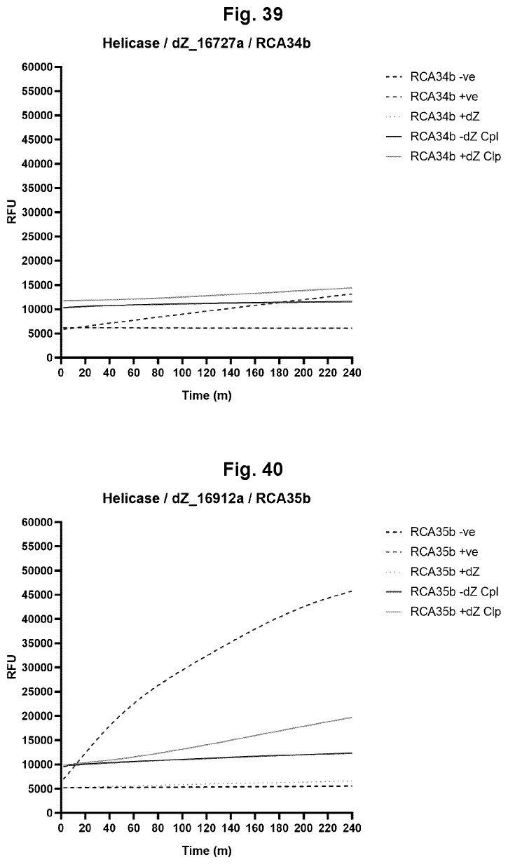

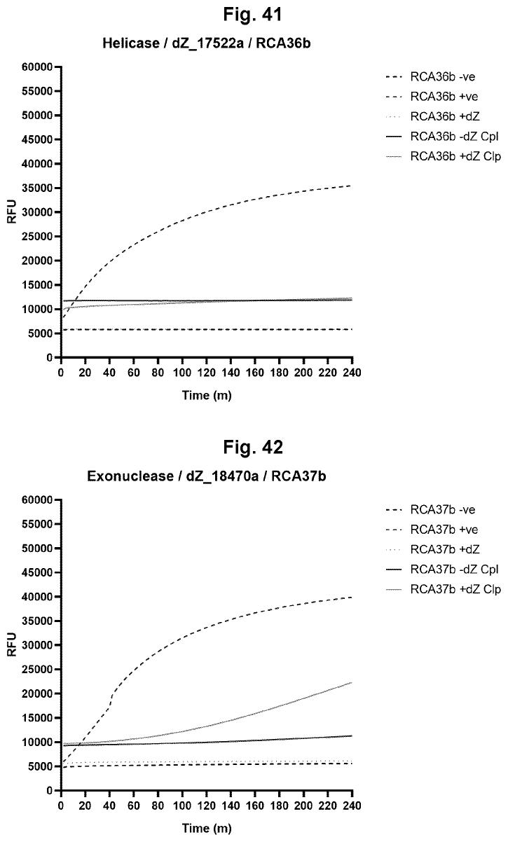

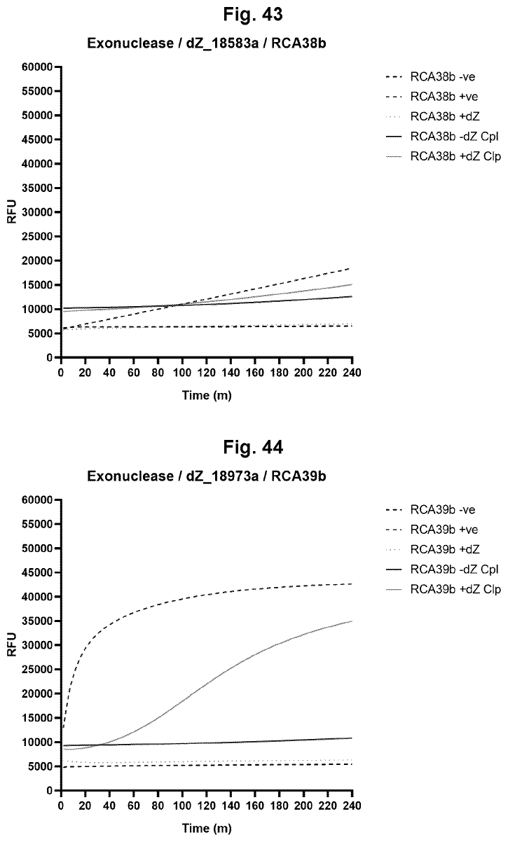

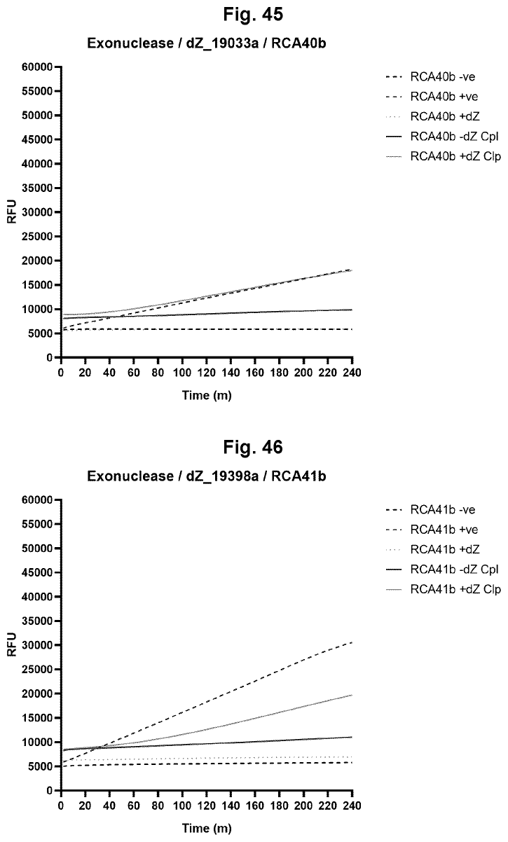

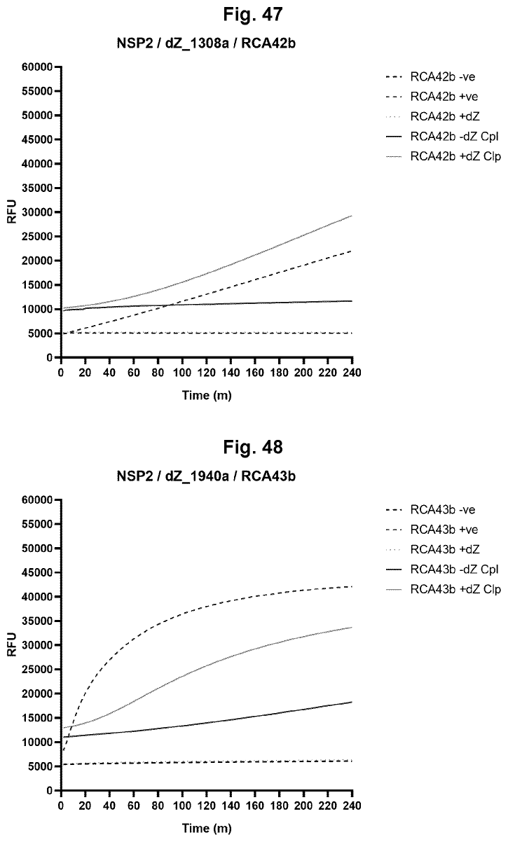

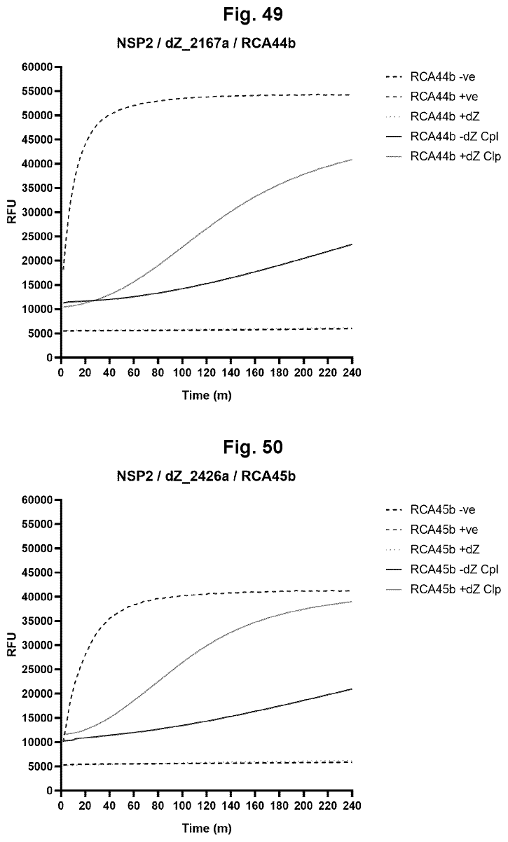

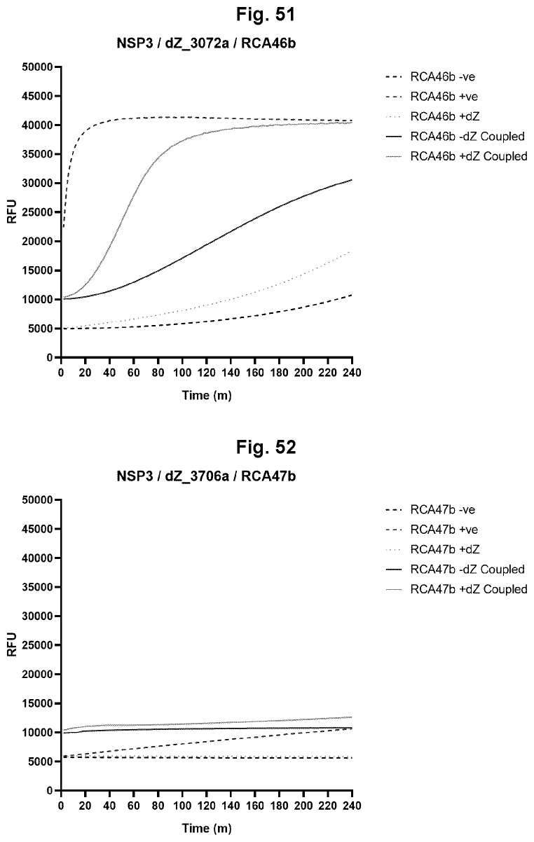

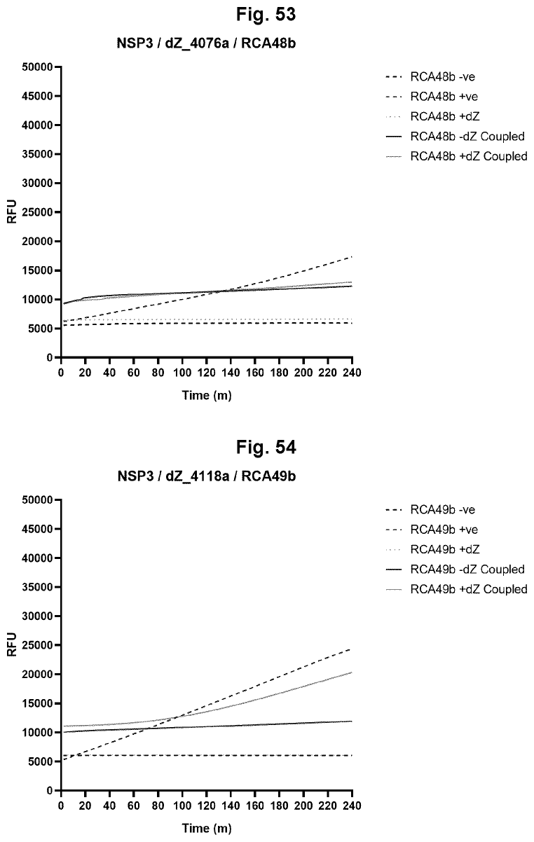

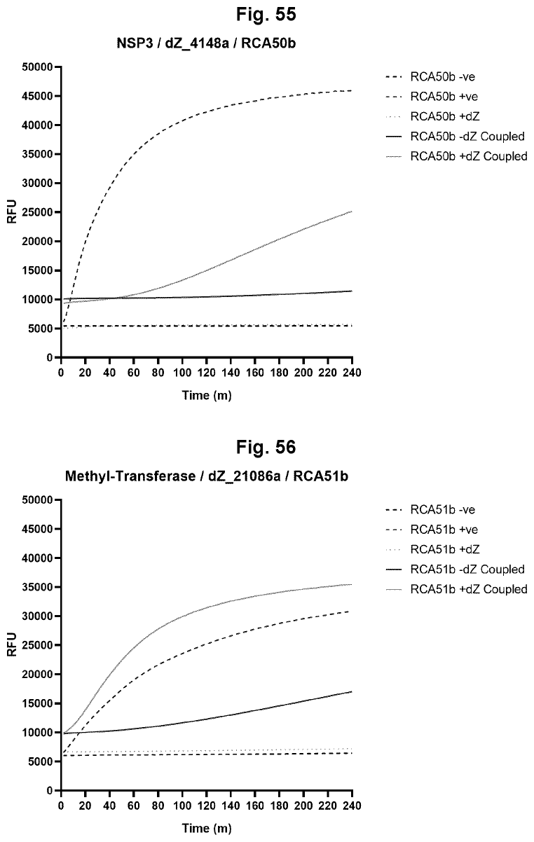

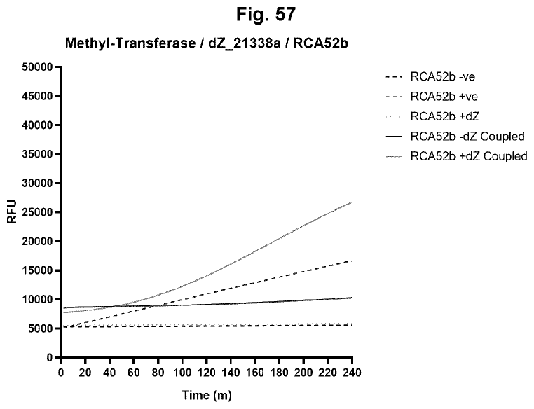

FIG. 23 to FIG. 27 show fluorescence results from coupled DNAzyme-RCA reactions targeting RdRp. FIG. 28 shows fluorescence results from coupled DNAzyme-RCA reactions targeting 3CL. FIG. 29 shows fluorescence results from coupled DNAzyme-RCA reactions targeting NSP1. FIG. 30 shows fluorescence results from coupled DNAzyme-RCA reactions targeting NSP6. FIG. 31 to FIG. 35 show fluorescence results from coupled DNAzyme-RCA reactions targeting NSP8. FIG. 36 and FIG. 37 show fluorescence results from coupled DNAzyme-RCA reactions targeting NSP15. FIG. 38 to FIG. 41 show fluorescence results from coupled DNAzyme-RCA reactions targeting helicase. FIG. 42 to FIG. 46 show fluorescence results from coupled DNAzyme-RCA reactions targeting exonuclease. FIG. 47 to FIG. 50 show fluorescence results from coupled DNAzyme-RCA reactions targeting NSP2. FIG. 51 to FIG. 55 show fluorescence results from coupled DNAzyme-RCA reactions targeting NSP3. FIGS. 56 and 57 shows fluorescence results from coupled DNAzyme-RCA reactions targeting methyltransferase.

### Example 4. RCA Product Detection Using a Lateral Flow Device

Detection of RCAP generated from using RNase I or DNAzyme-cleaved SARS-CoV-2 RNA as RCA primers in a lateral flow device (LFD) format can provide a rapid qualitative (yes/no) answer that is simple to read visually without specialized equipment. A lateral flow device is typically formed by lateral flow test strip with a sample pad and a conjugate pad on one end of the strip and an adsorption pad on the other. A test line providing the visualization area for a positive test result and a control line for visualizing functionality of the test may be located between the two ends of the strip. Given the simplicity of the LFD test, it should be appropriate for home use, eliminating the need for containment facilities, expensive equipment or skilled operators. This diagnostic platform device provides an unmet need for a rapid, low-cost test for COVID-19 and is applicable in low resource settings both in rural and urban settings for equitable testing.

Translation of RNA target binding and cleavage to detection on the LFD is done via RCAP facilitated release of a short DNA strand (denoted as bridging DNA or bDNA) from a bDNA/tDNA duplex (t: toehold) using the toehold DNA displacement mechanism.^{[13,14]} Briefly, the bDNA and tDNA in the duplex are not fully hybridized (i.e. these sequences are not completely complementarity) such that some amount of the tDNA sequence hangs off the end (i.e. the toehold). In the presence of the RCAP, the higher complementarity of the tDNA to the RCAP causes the bDNA/tDNA duplex to dissociate, releasing the bDNA. A portion of the free bDNA is designed to be complementary to an oligonucleotide sequence (denoted as cDNA1) attached to a gold nanoparticle (AuNP). The other portion of the bDNA is free to bind another complementary oligonucleotide sequence (denoted as cDNA2) attached to the surface of the LFD such that bDNA binding to the cDNA2 captures the bDNA/cDNA1/AuNP complex on the LFD.

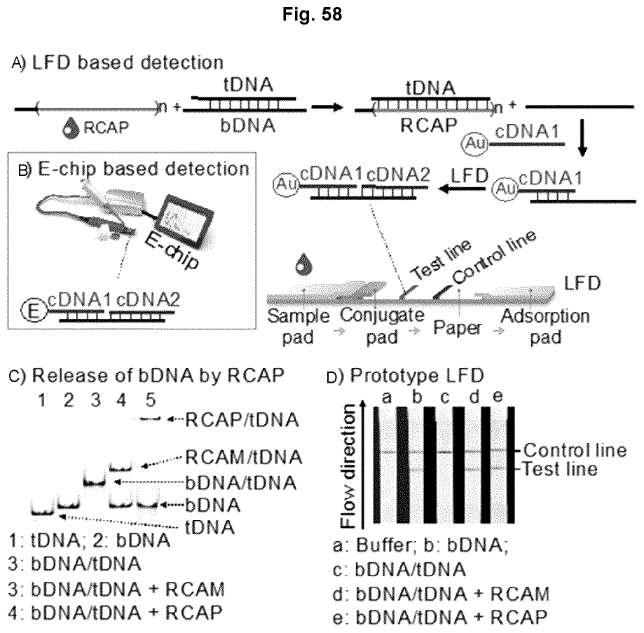

When an LFD modified with cDNA1 and cDNA2 is added to Vial 2 (already containing bDNA and tDNA) after RCA, the solution containing displaced bDNA will be flowed up the LFD (FIG. 58A). Flow of bDNA past a conjugate pad causes one end of bDNA to bind to cDNA1 modified with AuNP, which then moves further up the LFD for capture by cDNA2 printed at the test line. The assay also contains a control RNA to produce a control line demonstrating a successful test.

As RCA produces many repeating units in an RCAP per input RNA molecule, the method releases many bDNA per RNA cleavage by the DNAzyme. As such, bDNA concentration increases when there is a higher level of viral RNA to bridge more cDNA1 and cDNA2, producing a darker test line on the LFD.

The toehold mechanism can also be used to develop an electrochemical sensing assay where target-dependent current is measured by a portable potentiostat reader (FIG. 58B), in a design similar to the LFD except for (1) replacing AuNP with an electrochemical tag (denoted as cDNA1 labeled with E) and (2) immobilizing cDNA2 on an electrode chip such that capturing the released bDNA with cDNA2 produces an electronic signal.

This toehold-mechanism-to-LFD design allows for multiplexed assay format, where different regions of the genomic RNA are probed simultaneously to increase the test specificity.

### Methods

Synthesis of gold nanoparticles (GNPs): Gold nanoparticles of ˜20 nm diameter were synthesized in 100 mL volume. First, all glassware, including two sets of a necked round-bottom flask, stirrer bar, and condenser were washed with Aqua Regia (3:1 HCl: HNO₃) to remove all contaminants which can potentially lead to the aggregation of particles during synthesis or storage. Afterwards, all glasswares were washed with copious amounts of ddH₂O water and dried. Next, 100 mL of 2.2 mM sodium citrate was heated at 100° C. with a heating mantle in a 250 mL two-necked round-bottomed flask for 30 min under vigorous stirring. A cleaned condenser was equipped in one neck to prevent solvent evaporation during synthesis. The second neck was closed using a rubber septum. Once boiling commenced, 668 μL of HAuCl₄ (25 mM) was injected through the second neck. The color of the solution changed from yellow to dark blue and then to cherry red in 10 min. The heating at 100° C. was continued for a total of 25 min and then lowered to 90° C. for an additional 30 min. next, 668 μL of HAuCl₄ (25 mM) was injected again and heated for 30 min under vigorous stirring. The addition of of HAuCl₄ (25 mM) was repeated for two more rounds to produce ˜20 nm GNP (0.8 nM). The resulting suspension was analyzed using UV-Vis for their size and concentration.

Coupling of DNA with citrate capped AuNP: 600 μL of the gold nanoparticle (AuNP) suspension was taken in a glass vial. To this AuNP suspension, 20 μL (100 μM stock) of thiol-DNA (control and test DNA were coupled in separate vials) was added to the above vial followed by 380 μL water to adjust the volume up to 1.0 mL. After brief vortex, the suspension was incubated at room temperature for 24 h. 10 μL of Tris-HCl (1 M, pH.7.5) and 90 μL NaCl (1 M) were mixed in the suspension and incubated for another 24 h. 5 μL of Tris-HCl (1 M, pH.7.5) and 50 μL NaCl (1 M) were added and the reaction was incubated at room temperature for another 24 h. Finally, the AuNP suspension was centrifuged at 14000 rpm (˜21000 g) at room temperature for 20 min. The clear supernatant was discarded and the particles were re-dispersed again with 500 μL buffer (20 mM, pH 7.5, NaCl 150 mM). The washing step was repeated one more time and resuspended in 500 uL buffer (20 mM, pH 7.5, NaCl 150 mM, 250 mM sucrose) and this ready to use suspension was stored at 4° C.

Fabrication of LFD: TL-DNA (test line DNA) and CL-DNA (control line DNA) were printed on nitrocellulose paper (NCP) as follows: 5 μM of streptavidin (Millipore, Burlington, Canada) and 25 μM of each of TL- and CL-DNA were individually mixed in 200 μL of PBS (pH 7.4) and incubated at room temperature for 30 min. After incubation, the streptavidin-DNA conjugate was passed through centrifugal column (Amicon @Ultra-0.5 mL, Millipore) of 30K molecular cut off size for 10 min at 14000 g. The conjugate was washed twice with 200 μL of PBS. After washing, the concentrated streptavidin-DNA was recovered by placing the filter device upside down into a clean micro centrifuge tube and centrifugation at 1000 g for 2 min. The recovered streptavidin-DNA was diluted to a final volume of 100 μL using PBS buffer. Nitrocellulose paper (NCP, Immunopore FP grade from GE Healthcare) was cut into a 25×300 mm piece. Control and test lines (0.5 mm diameter) were printed on the NCP ˜22 mm below the top edge with 5 mm inter line distance using a Scienion sciflexarrayer s5 non-contact microarray printer. After printing, the NCP was air dried for 30 min. The printed NCP obtained in the above step was attached onto the backing card for cutting and handling. Meanwhile, the absorbent pad (Ahlstrome grade 270) was cut into 20×300 mm in size and attached on the backing cardjust above the prineted lines of NCP obtained in the above step. The assembled pieces were then cut into 4 mm diameter (wide) by CM5000 Guillotine Cutter (BioDot). Glass fiber was used as sample pad and conjugate pads both in 4×10 mm size. Before cutting the sample pad glass fibre, it was immersed in the sample pad buffer (Tris-HCl 25 mM, pH 7.5, including 300 mM NaCl, 0.1% SDS and dried for 2 hrs. In the conjugate pad glass fibre, mixture of gold conjugates (mixture of equivalent amount of both test and control) was pipetted twice and dried at room temperature before cutting. Next, the glass fibres were cut into 4×10 mm size and attached in the designated location (bottom of the LFD) with 0.5 mm overlap of each pad. This ready to use dipstick device was stored at room temperature until use.

RCA: sequences design and LFD test: Four DNA sequences were designed (Table 6): 1) a template for converting into a circle, 2) a ligation template to make the circle, 3) a toehold sequence (tDNA) and 4) a bridging sequence (bDNA). tDNA was completely complementary to a part of the RCA product while tDNA and bDNA are partially complementary to each other. In this case, if there is no RCA product tDNA and bDNA will remain as duplex and will not bind to the test AuNP-DNA and no line will be generated in the test line. If there is RCA product, the tDNA will be hybridized with the RCA product releasing the bDNA available for binding to TL-DNA and be captured in the test line generating a red line. The duplex between tDNA and bDNA was native PAGE purified so that there is no free bDNA to generate false positive results.

Preparing the DNA circle: One nanomole of circular template was phosphorylated at the 5′-end by treating with 10 U of PNK in presence of 10 mM ATP and 1×PNK buffer A for 35 min at 37 C in 100 uL volume. The reaction was quenched by heating at 90 C for 5 min. Next, an equivalent amount of the ligation template was added to the reaction mixture and heated at 90 C for 1 min. To this mixture sequentially added 30 uL PEG4000, 30 uL of 10×T4 DNA ligase buffer and 5 uL of T4 DNA ligase. The volume was adjusted to 300 uL by ddH2O. The ligation reaction was conducted at room temperature for 1 h. The circle was isolated by ethanol precipitation and purified by 10% denaturing PAGE (dPAGE), recovered from the gel using elution buffer (10 mM Tris-HCl, pH 7.5, 100 mM NaCl, 1 mM EDTA)), dissolved in ddH2O, quantified by UV and stored at −20° C. until use.

RCA and LFD test: RCA reaction was conducted in 100 uL volume in 1×Phi29DP buffer including 10 nM each of circle and primers, 0.5 mM dNTPs, 50 nM of tDNA-bDNA duplex for 10 min at room temperature. LFD was directly dipped into this reaction mixture and allowed to flow for min before taking the photograph (strip e in FIG. 19D). The control tests for the LFDs were: a) in buffer alone without any DNA, b) bDNA alone (positive control), c) bDNA-tDNA duplex only and d) bDNA-tDNA duplex in presence of the monomeric RCA product.

### Results

FIG. 58C shows toehold-mediated bDNA displacement using gel electrophoresis. Both tDNA (lane 1) and bDNA (lane 2) were fluorophore-labeled. The bDNA was initially engaged into the bDNA/tDNA duplex (lane 3). Upon mixing with either synthetic RCAP monomer (RCAM, a positive control; lane 4) or RCAP (lane 5), bDNA was displaced. FIG. 58D shows an LFD in which the presence of RCAM (strip d) or RCAP (strip e) clearly led to a strong red test line (other strips are controls). The signal generation only took ˜5 min. Counting RNA cleavage (10 min), RCA (10 min) and signal development on LFD (˜5 min), the entire process took less than 30 min, which would be further reduced when HRCA is incorporated.

### Example 5. RCA Detection Using RCA-Coupled Nicking

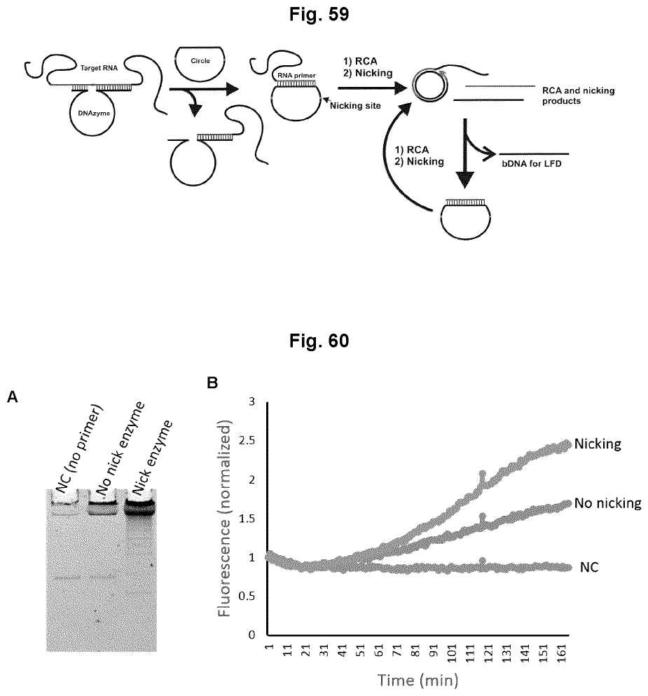

An alternative route for generating bDNA is depicted in the schematic representation of bDNA generation by DNAzyme initiated RCA coupled nicking enzyme (FIG. 59). Target RNA is first cleaved by DNAzyme. The 5′ fragment of the cleaved product is used as primer for initiating RCA, which is conducted in the presence of nicking enzyme (Nb.BbvCI). The circle contained two nicking sites so that two fragments will be generated after one successful round of RCA and nicking. One nicking product will serve as a primer of a second CDT, or the same CDT (in this case, an excess amount of CDT needs to be added) and another fragment will serve as bDNA. Overtime, more and more bDNA will accumulate to generate strong signal in the test line of a LFD.

### Methods

The RCA-coupled nicking was tested using a CDT with nicking sites (Nick-CDT) and RCA primer (Nick-primer) as shown in Table 7. Similarly, CDTs with nicking sites. First, the ligation reaction to make circle was conducted in 30 μL reaction volume in 1× splintR ligase buffer (NEB) at 37° C. for 20 min in the presence of 33 nM of N1PdL2 (5′ phosphorylated), 1 nM of target RNA and 12 units of SplintR ligase. Next, to this reaction mixture, sequentially 1 μL of primer (1 μL stock), 5 μL 10×Phi29 buffer, 2.5 μL dNTPs (10 mM stock), 0.5 μL BSA (20 mg/mL stock), 5 units of Phi29 DNA polymerase and 5 units of Nb.BbvCI nicking enzyme were added. The reaction volume was adjusted to 50 μL with autoclaved ddH2O and the reaction as conducted at 30° C. for 30 min. Two control experiments were conducted. In the first control, ligation was conducted in the absence of RCA-primer whereas in the second control, nicking enzyme was omitted. The reaction mixtures were analyzed by denaturing PAGE. Similarly, target RNA triggered RCA-coupled nicking can be performed using CTDs complementary to target RNA, such as n1 RNA using sequences provided in Table 7.

### Results

The results showed that the RCA in the presence of nicking enzyme produced significantly higher RCA product compared to the RCA reaction that was conducted in the absence of nicking enzyme (FIG. 60A).

FIG. 60B shows that this was further demonstrated by real time fluorescence measurement by plate reader (Tecan M100). In this case, the ligation reaction was conducted in 30 μL volume in the same way as described above for dPAGE. For fluorescence monitoring, the RCA reaction volume was increased to 100 μL and the other reagents (10 uL 10×Phi29 buffer, 10 Units of Phi29 DNA polymerase, 10 units of nicking enzyme, and 1 μL of BSA) were doubled. Additionally, 0.5×SYBR™ gold (Invitrogen) was added for fluorescence signal generation. The reactions were conducted in a 96 well black plate, clear bottom with the wavelength set up: excitation 495 nm and emission 537 nm.

### Example 6. Multiplexing with Non-RNA Targets

This DNAzyme-based LFD platform can be further multiplexed by linking with other functional nucleic acids, such as DNA aptamers^{[15]} for the detection of specific SARS-CoV-2 protein biomarkers (e.g. S1, N and RdRP proteins). As nucleic acids, aptamers for these target proteins can be integrated with the RCA detection platform to develop an aptamer-initiated RCA assay.^{[16,17]} Linking protein-aptamer binding to RCA can be done using a method, “digestion-initiated RCA”,^{[17]} that makes use of the ability for Phi29DP to carry out 3′-5′ exonucleolytic degradation of single-stranded DNA, in addition to polymerization and strand displacement.^{[18]} Briefly, it uses a tripartite DNA assembly made of a CDT, a pre-primer (PP) and an aptamer probe (AP). Their sequences are designed to allow the formation of two DNA duplexes, one involving the CDT and the 5′-end of the PP and other involving the 3′-end of the PP and the 5′-end of the AP. In the absence of the target, the formation of the two duplexes prevents RCA by Phi29DP. With the target, the AP makes a partner switch from the PP to the target. This event produces a single-stranded region in the PP, which is trimmed by Phi29DP, converting the PP into a mature primer (MP) for RCA. Detection of the RCAP generated from aptamer detection can then be designed similarly using the toehold mechanism integrated with a simple LFD readout such that a single POCT can detect both viral RNA and viral proteins simultaneously. This simple integration allows for testing of multiple different targets for increased accuracy.

The POCT systems described herein allow for the rapid detection of SARS-CoV-2 that is highly specific and sensitive both analytically and clinically, simple to use, produced with easy to obtain reagents, cost-efficient and performed at room temperature with no extraction step. This can make such POCTs available for wide-spread deployment from common to non-standard and remote testing locations, including screening at places of employment, ports of entry, or at home, to improve patient-centered care. The simplicity of a one-stop sample-to-answer test that can be used anywhere by anyone will be crucial to drive down the spread of the virus, allow more rapid contact tracing, and thus limit outbreaks at an earlier stage.

While the present disclosure has been described with reference to examples, it is to be understood that the scope of the claims should not be limited by the embodiments set forth in the examples, but should be given the broadest interpretation consistent with the description as a whole.

TABLE 1
Oligonucleotide sequences.

| Sequence | | |
|---|---|---|
| ID | | |
| Number | Name | Sequence (5′→3′) |
| 1 | n1 RNA | GGGAUGUCUGAUAAUGGACCCCAAAAUCAG |
| | | CGAAAUGCACCCCGCAUUACGUUUGGUGGA |
| | | CCCUCAGAUUCAACUGGCAGUAACCAGAAU |
| | | GGAGAACGCAGUGGG |
| 2 | n2 RNA | GGGUAUGGGUUGCAACUGAGGGAGCCUUGA |
| | | AUACACCAAAAGAUCACAUUGGCACCCGCA |
| | | AUCCUGCUAACAAUGCUGCAAUCGUGCUAC |
| | | AACUUCCUCAAGG |
| 3 | n3 RNA | GGGCCAGGAACUAAUCAGACAAGGAACUGA |
| | | UUACAAACAUUGGCCGCAAAUUGCACAAUU |
| | | UGCCCCCAGCGCUUCAGCGUUCUUCGGAAU |
| | | GUCGCGCAUUGGC |
| 4 | nCov_ORF1ab_13470_T7_R | GGGUUUGCGGUGUAAGUGCAGCCCGUCUUA |
| | NA | CACCGUGCGGCACAGGCACUAGUACUGAUG |
| | | UCGUAU |
| 5 | nCov_ORF1ab_13513_T7_R | GGGCACUAGUACUGAUGUCGUAUACAGGGC |
| | NA | UUUUGACAUCUACAAUGAUAAAGUAGCUGG |
| | | UUUUGC |
| 6 | nCov_S_24356_T7_RNA | GGGCAAAAUUCAAGACUCACUUUCUUCCAC |
| | | AGCAAGUGCACUUGGAAAACUUCAAGAUGU |
| | | GGUCAA |
| 7 | nCov_S 24526 T7 RNA | GGGCUGAAGUGCAAAUUGAUAGGUUGAUCA |
| | | CAGGCAGACUUCAAAGUUUGCAGACAUAUG |
| | | UGACUC |
| 8 | nCov_E_26286_T7_RNA | GGGUAAUAGCGUACUUCUUUUUCUUGCUUU |
| | | CGUGGUAUUCUUGCUAGUUACACUAGCCAU |
| | | CCUUACUG |
| 9 | nCov_E_26329_T7_RNA | GGGUUACACUAGCCAUCCUUACUGCGCUUC |
| | | GAUUGUGUGCGUACUGCUGCAAUAUUGUUA |
| | | ACGUGAG |
| 10 | N_CDCn1_GU1_1023b | CCACCAAAGGCTAGCTACAACGAGTAATGC |
| 11 | N_CDCn1_GU1_1023c | GGGTCCACCAAAGGCTAGCTACAACGAGTA |
| | (GU1c) | ATGC |
| 12 | N_CDCn1_GU1_1023d | AGGGTCCACCAAAGGCTAGCTACAACGAGT |
| | | AATGCG |
| 13 | N_CDCn1_GU1_1023e | GAGGGTCCACCAAAGGCTAGCTACAACGAG |
| | | TAATGCG |
| 14 | N_CDCn1_GU1_1023f | CTGAGGGTCCACCAAAGGCTAGCTACAACG |
| | | AGTAATGCG |
| 15 | N_CDCn1_GU1_1023g | TGAATCTGAGGGTCCACCAAAGGCTAGCTA |
| | | CAACGAGTAATGCG |
| 16 | N_CDCn1_GU1_1023_DNA | TGCACCCCGCATTACG |
| 17 | N_CDCn1_GU3_1023b | TCTGGTTAGGCTAGCTACAACGATGCCAGT |
| 18 | N_CDCn1_GU3_1023c | TCCATTCTGGTTAGGCTAGCTACAACGATG |
| | | CCAGT |
| 19 | N_CDCn1_GU3_1023f | TTCTCCATTCTGGTTAGGCTAGCTACAACG |
| | | ATGCCAGTT |
| 20 | N_CDCn1_GU3_1023_DNA | CAGATTCAACTGGCAG |
| 21 | N_CDCn2_AU6_1023b | CAATGTGAGGCTAGCTACAACGACTTTTGG |
| 22 | N_CDCn2_AU6_1023f | GCGGGTGCCAATGTGAGGCTAGCTACAACG |
| | | ACTTTTGGT |
| 23 | N_CDCn2_AU6_1023_DNA | TGAATACACCAAAAGA |
| 24 | N_CDCn2_AU7_1023b | TAGCAGGAGGCTAGCTACAACGATGCGGGT |
| 25 | N_CDCn2_AU7_1023f | AGCATTGTTAGCAGGAGGCTAGCTACAACG |
| | | ATGCGGGTG |
| 26 | N_CDCn2_AU7_1023_DNA | ACATTGGCACCCGCAA |
| 27 | N_CDCn3_AU10_1023b | GCGGCCAAGGCTAGCTACAACGAGTTTGTA |
| 28 | N_CDCn3_AU10_1023f | TGCAATTTGCGGCCAAGGCTAGCTACAACG |
| | | AGTTTGTAA |
| 29 | N_CDCn3_AU10_1023_DN | GAACTGATTACAAACA |
| | A | |
| 30 | N_CDCn3_GU5_1023b | CCGAAGAAGGCTAGCTACAACGAGCTGAAG |
| 31 | N_CDCn3_GU5_1023f | GCGACATTCCGAAGAAGGCTAGCTACAACG |
| | | AGCTGAAGC |
| 32 | N_CDCn3_GU5_1023_DNA | CCCCAGCGCTTCAGCG |
| 33 | ORF1ab_CCDC_GU4_1023b | GTGTAAGAGGCTAGCTACAACGAGGGCTGC |
| 34 | ORF1ab_CCDC_GU4_1023f | GCCGCACGGTGTAAGAGGCTAGCTACAACG |
| | | AGGGCTGCA |
| 35 | ORF1ab_CCDC_GU4_1023_ | GTGTAAGTGCAGCCCG |
| | DNA | |
| 36 | ORF1ab_CCDC_AU3_1023b | ATTGTAGAGGCTAGCTACAACGAGTCAAAA |
| 37 | ORF1ab_CCDC_AU3_1023f | ACTTTATCATTGTAGAGGCTAGCTACAACG |
| | | AGTCAAAAG |
| 38 | ORF1ab _CCDC_AU3_1023_ | TACAGGGCTTTTGACA |
| | DNA | |
| 39 | S_Japan GU1_1023b | CAAGTGCAGGCTAGCTACAACGATTGCTGT |
| 40 | S_Japan_GU1_1023f | AAGTTTTCCAAGTGCAGGCTAGCTACAACG |
| | | ATTGCTGTG |
| 41 | S_Japan_GU1_1023_DNA | TTTCTTCCACAGCAAG |
| 42 | S_Japan_AU11_1023b | GCCTGTGAGGCTAGCTACAACGACAACCTA |
| 43 | S_Japan_AU11_1023f | TGAAGTCTGCCTGTGAGGCTAGCTACAACG |
| | | ACAACCTAT |
| 44 | S_Japan_AU11_1023_DNA | CAAATTGATAGGTTGA |
| 45 | E_Germany AU3_1023b | AGCAAGAAGGCTAGCTACAACGAACCACGA |
| 46 | E_Germany_AU3_1023f | GTGTAACTAGCAAGAAGGCTAGCTACAACG |
| | | AACCACGAA |
| 47 | E_Germany_AU3_1023_DN | TCTTGCTTTCGTGGTA |
| | A | |
| 48 | E_Germany_AU5_1023b | GCACACAAGGCTAGCTACAACGACGAAGCG |
| 49 | E_Germany_AU5_1023f | AGCAGTACGCACACAAGGCTAGCTACAACG |
| | | ACGAAGCGC |
| 50 | E_Germany_AU5_1023_DN | CCTTACTGCGCTTCGA |
| | A | |
| 51 | N_CDCn2-3_M1_1023b | CAATGTGAGGCTAGCTACAACGTCTTTTGG |
| | | TGTATTCAGGATCCGCGGCCAAGGCTAGCT |
| | | ACAACGTGTTTGTAATCAGTTC |
| 52 | M1_Lig_Tmp | CCTCACATTGGAACTGATTA |
| 53 | M1_n2_DNA | TGAATACACCAAAAGA |
| 54 | M1_n3_DNA | GAACTGATTACAAACA |
| 55 | RCA1 | CGTAATGCGGGGTGCAGGATCCTGTTTGTA |
| | | ATCAGTTCCTCTTTTGGTGTATTCA |
| 56 | RCA1_Lig_Tmp | CCGCATTACGTGAATACACC |
| 57 | RCA2 | CTGCCAGTTGAATCTGGGATCCTTGCGGGT |
| | | GCCAATGTCGCTGAAGCGCTGGGG |
| 58 | RCA2_Lig_Tmp | CAACTGGCAGCCCCAGCGCT |
| 59 | RCA3 | CGGGCTGCACTTACACGGATCCCTTGCTGT |
| | | GGAAGAAATACCACGAAAGCAAGA |
| 60 | RCA3_Lig_Tmp | GTGCAGCCCGTCTTGCTTTC |
| 61 | RCA4 | TGTCAAAAGCCCTGTAGGATCCTCAACCTA |
| | | TCAATTTGTCGAAGCGCAGTAAGG |
| 62 | RCA4_Lig_Tmp | GCTTTTGACACCTTACTGCG |
| 63 | dZ_28692a | GTGATCTTTTGGTGTAGGCTAGCTACAACG |
| | | ATCAAGGCT |
| 64 | dZ_28734a | TAGCACGATTGCAGCAGGCTAGCTACAACG |
| | | ATGTTAGCA |
| 65 | dZ_28771a | AGAAGCCTTTTGGCAAGGCTAGCTACAACG |
| | | AGTTGTTCC |
| 66 | dZ_28851a | AGTTGAATTTCTTGAAGGCTAGCTACAACG |
| | | ATGTTGCGA |
| 67 | dZ_21744a | ATGGAACCAAGTAACAGGCTAGCTACAACG |
| | | ATGGAAAAG |
| 68 | dZ_21768a | ATTGGTCCCAGAGACAGGCTAGCTACAACG |
| | | AGTATAGCA |
| 69 | dZ_21969a | CAAAAATGGATCATTAGGCTAGCTACAACG |
| | | AAAAATTGA |
| 70 | dZ_22161a | AGAATATATTTTAAAAGGCTAGCTACAACG |
| | | AAACCATCA |
| 71 | dZ_22614a | CTTCCTGTTCCAAGCAGGCTAGCTACAACG |
| | | AAAACAGAT |
| 72 | dZ_23847a | TTAAAGCACGGTTTAAGGCTAGCTACAACG |
| | | ATGTGTACA |
| 73 | dZ_24178a | ACAGTGCAGAAGTGTAGGCTAGCTACAACG |
| | | ATGAGCAAT |
| 74 | dZ_24468a | TGAAATTGCACCAAAAGGCTAGCTACAACG |
| | | ATGGAGCTA |
| 75 | dZ_24710a | GACTGAGGGAAGGACAGGCTAGCTACAACG |
| | | AAAGATGAT |
| 76 | dZ_25097a | TCAATTTCTTTTTGAAGGCTAGCTACAACG |
| | | AGTTTACAA |
| 77 | dZ_25271a | CTACAGCAACTGGTCAGGCTAGCTACAACG |
| | | AACAGCAAA |
| 78 | dZ_13533a | TGTCAAAAGCCCTGTAGGCTAGCTACAACG |
| | | AACGACATC |
| 79 | dZ_13625a | ATCAATTAAATTGTCAGGCTAGCTACAACG |
| | | ACTTCGTCC |
| 80 | dZ_13726a | AAGTCATGTTTAGCAAGGCTAGCTACAACG |
| | | AAGCTGGAC |
| 81 | dZ_14172a | CCCTGGTCAAGGTTAAGGCTAGCTACAACG |
| | | AATAGGCAT |
| 82 | dZ_14578a | CCAGAAGCAGCGTGCAGGCTAGCTACAACG |
| | | AAGCAGGGT |
| 83 | dZ_14829a | GTTGTCTGATATCACAGGCTAGCTACAACG |
| | | AATTGTTGG |
| 84 | dZ_14984a | ACTCATTGAATCATAAGGCTAGCTACAACG |
| | | AAAAGTCTA |
| 85 | dZ_15029a | GACATTACGTTTTGTAGGCTAGCTACAACG |
| | | AATGCGAAA |
| 86 | dZ_15165a | CGGCTATTGATTTCAAGGCTAGCTACAACG |
| | | AAATTTTTG |
| 87 | dZ_15202a | TTGCTTGTTCCAATTAGGCTAGCTACAACG |
| | | ATACAGTAG |
| 88 | dZ_15282a | GGATAATCCCAACCCAGGCTAGCTACAACG |
| | | AAAGGTGAG |
| 89 | dZ_15506a | AAAAACACTATTAGCAGGCTAGCTACAACG |
| | | AAAGCAGTT |
| 90 | dZ_15439a | GAACCGCCACACATGAGGCTAGCTACAACG |
| | | ACATTTCAC |
| 91 | dZ_15703a | TCAGAGAGTATCATCAGGCTAGCTACAACG |
| | | ATGAGAAAT |
| 92 | dZ_15921a | CTGGGTAAGGAAGGTAGGCTAGCTACAACG |
| | | AACATAATC |
| 93 | dZ_26666a | AGGAAAATTAACTTAAGGCTAGCTACAACG |
| | | ATATATACA |
| 94 | dZ_26718a | TAAACAGCAGCAAGCAGGCTAGCTACAACG |
| | | AAAAACAAG |
| 95 | dZ_26874a | GGCACGTTGAGAAGAAGGCTAGCTACAACG |
| | | AGTTAGTTT |
| 96 | dZ_27137a | AATGGTCTGTGTTTAAGGCTAGCTACAACG |
| | | ATTATAGTT |
| 97 | Nucleocapsid Full | GGGAUGUCUGAUAAUGGACCCCAAAAUCAG |
| | | CGAAAUGCACCCCGCAUUACGUUUGGUGGA |
| | | CCCUCAGAUUCAACUGGCAGUAACCAGAAU |
| | | GGAGAACGCAGUGGGGCGCGAUCAAAACAA |
| | | CGUCGGCCCCAAGGUUUACCCAAUAAUACU |
| | | GCGUCUUGGUUCACCGCUCUCACUCAACAU |
| | | GGCAAGGAAGACCUUAAAUUCCCUCGAGGA |
| | | CAAGGCGUUCCAAUUAACACCAAUAGCAGU |
| | | CCAGAUGACCAAAUUGGCUACUACCGAAGA |
| | | GCUACCAGACGAAUUCGUGGUGGUGACGGU |
| | | AAAAUGAAAGAUCUCAGUCCAAGAUGGUAU |
| | | UUCUACUACCUAGGAACUGGGCCAGAAGCU |
| | | GGACUUCCCUAUGGUGCUAACAAAGACGGC |
| | | AUCAUAUGGGUUGCAACUGAGGGAGCCUUG |
| | | AAUACACCAAAAGAUCACAUUGGCACCCGC |
| | | AAUCCUGCUAACAAUGCUGCAAUCGUGCUA |
| | | CAACUUCCUCAAGGAACAACAUUGCCAAAA |
| | | GGCUUCUACGCAGAAGGGAGCAGAGGCGGC |
| | | AGUCAAGCCUCUUCUCGUUCCUCAUCACGU |
| | | AGUCGCAACAGUUCAAGAAAUUCAACUCCA |
| | | GGCAGCAGUAGGGGAACUUCUCCUGCUAGA |
| | | AUGGCUGGCAAUGGCGGUGAUGCUGCUCUU |
| | | GCUUUGCUGCUGCUUGACAGAUUGAACCAG |
| | | CUUGAGAGCAAAAUGUCUGGUAAAGGCCAA |
| | | CAACAACAAGGCCAAACUGUCACUAAGAAA |
| | | UCUGCUGCUGAGGCUUCUAAGAAGCCUCGG |
| | | CAAAAACGUACUGCCACUAAAGCAUACAAU |
| | | GUAACACAAGCUUUCGGCAGACGUGGUCCA |
| | | GAACAAACCCAAGGAAAUUUUGGGGACCAG |
| | | GAACUAAUCAGACAAGGAACUGAUUACAAA |
| | | CAUUGGCCGCAAAUUGCACAAUUUGCCCCC |
| | | AGCGCUUCAGCGUUCUUCGGAAUGUCGCGC |
| | | AUUGGCAUGGAAGUCACACCUUCGGGAACG |
| | | UGGUUGACCUACACAGGUGCCAUCAAAUUG |
| | | GAUGACAAAGAUCCAAAUUUCAAAGAUCAA |
| | | GUCAUUUUGCUGAAUAAGCAUAUUGACGCA |
| | | UACAAAACAUUCCCACCAACAGAGCCUAAA |
| | | AAGGACAAAAAGAAGAAGGCUGAUGAAACU |
| | | CAAGCCUUACCGCAGAGACAGAAGAAACAG |
| | | CAAACUGUGACUCUUCUUCCUGCUGCAGAU |
| | | UUGGAUGAUUUCUCCAAACAAUUGCAACAA |
| | | UCCAUGAGCAGUGCUGACUCAACUCAGGCC |
| | | UAA |
| 98 | RdRp 13469/14676 | GGGUUUGCGGUGUAAGUGCAGCCCGUCUUA |
| | | CACCGUGCGGCACAGGCACUAGUACUGAUG |
| | | UCGUAUACAGGGCUUUUGACAUCUACAAUG |
| | | AUAAAGUAGCUGGUUUUGCUAAAUUCCUAA |
| | | AAACUAAUUGUUGUCGCUUCCAAGAAAAGG |
| | | ACGAAGAUGACAAUUUAAUUGAUUCUUACU |
| | | UUGUAGUUAAGAGACACACUUUCUCUAACU |
| | | ACCAACAUGAAGAAACAAUUUAUAAUUUAC |
| | | UUAAGGAUUGUCCAGCUGUUGCUAAACAUG |
| | | ACUUCUUUAAGUUUAGAAUAGACGGUGACA |
| | | UGGUACCACAUAUAUCACGUCAACGUCUUA |
| | | CUAAAUACACAAUGGCAGACCUCGUCUAUG |
| | | CUUUAAGGCAUUUUGAUGAAGGUAAUUGUG |
| | | ACACAUUAAAAGAAAUACUUGUCACAUACA |
| | | AUUGUUGUGAUGAUGAUUAUUUCAAUAAAA |
| | | AGGACUGGUAUGAUUUUGUAGAAAACCCAG |
| | | AUAUAUUACGCGUAUACGCCAACUUAGGUG |
| | | AACGUGUACGCCAAGCUUUGUUAAAAACAG |
| | | UACAAUUCUGUGAUGCCAUGCGAAAUGCUG |
| | | GUAUUGUUGGUGUACUGACAUUAGAUAAUC |
| | | AAGAUCUCAAUGGUAACUGGUAUGAUUUCG |
| | | GUGAUUUCAUACAAACCACGCCAGGUAGUG |
| | | GAGUUCCUGUUGUAGAUUCUUAUUAUUCAU |
| | | UGUUAAUGCCUAUAUUAACCUUGACCAGGG |
| | | CUUUAACUGCAGAGUCACAUGUUGACACUG |
| | | ACUUAACAAAGCCUUACAUUAAGUGGGAUU |
| | | UGUUAAAAUAUGACUUCACGGAAGAGAGGU |
| | | UAAAACUCUUUGACCGUUAUUUUAAAUAUU |
| | | GGGAUCAGACAUACCACCCAAAUUGUGUUA |
| | | ACUGUUUGGAUGACAGAUGCAUUCUGCAUU |
| | | GUGCAAACUUUAAUGUUUUAUUCUCUACAG |
| | | UGUUCCCACCUACAAGUUUUGGACCACUAG |
| | | UGAGAAAAAUAUUUGUUGAUGGUGUUCCAU |
| | | UUGUAGUUUCAACUGGAUACCACUUCAGAG |
| | | AGCUAGGUGUUGUACAUAAUCAGGAUGUAA |
| | | ACUUACAUAGCUCUAGACUUAGUUUUAAGG |
| | | AAUUACUUGUGUAUGCUGCUGACCCUGCUA |
| | | UGCACGCUGCUUCUGGUAAUCUAUUACUAG |
| | | AUAAACGCACUACGUGCUUUUCAGUAGCUG |
| | | CACUUACUAACAAUGUUGCUUUUCAAACUG |
| | | UCAAACCC |
| 99 | RdRp 14793/16197 | GGGCUCAGGAUGGUAAUGCUGCUAUCAGCG |
| | | AUUAUGACUACUAUCGUUAUAAUCUACCAA |
| | | CAAUGUGUGAUAUCAGACAACUACUAUUUG |
| | | UAGUUGAAGUUGUUGAUAAGUACUUUGAUU |
| | | GUUACGAUGGUGGCUGUAUUAAUGCUAACC |
| | | AAGUCAUCGUCAACAACCUAGACAAAUCAG |
| | | CUGGUUUUCCAUUUAAUAAAUGGGGUAAGG |
| | | CUAGACUUUAUUAUGAUUCAAUGAGUUAUG |
| | | AGGAUCAAGAUGCACUUUUCGCAUAUACAA |
| | | AACGUAAUGUCAUCCCUACUAUAACUCAAA |
| | | UGAAUCUUAAGUAUGCCAUUAGUGCAAAGA |
| | | AUAGAGCUCGCACCGUAGCUGGUGUCUCUA |
| | | UCUGUAGUACUAUGACCAAUAGACAGUUUC |
| | | AUCAAAAAUUAUUGAAAUCAAUAGCCGCCA |
| | | CUAGAGGAGCUACUGUAGUAAUUGGAACAA |
| | | GCAAAUUCUAUGGUGGUUGGCACAACAUGU |
| | | UAAAAACUGUUUAUAGUGAUGUAGAAAACC |
| | | CUCACCUUAUGGGUUGGGAUUAUCCUAAAU |
| | | GUGAUAGAGCCAUGCCUAACAUGCUUAGAA |
| | | UUAUGGCCUCACUUGUUCUUGCUCGCAAAC |
| | | AUACAACGUGUUGUAGCUUGUCACACCGUU |
| | | UCUAUAGAUUAGCUAAUGAGUGUGCUCAAG |
| | | UAUUGAGUGAAAUGGUCAUGUGUGGCGGUU |
| | | CACUAUAUGUUAAACCAGGUGGAACCUCAU |
| | | CAGGAGAUGCCACAACUGCUUAUGCUAAUA |
| | | GUGUUUUUAACAUUUGUCAAGCUGUCACGG |
| | | CCAAUGUUAAUGCACUUUUAUCUACUGAUG |
| | | GUAACAAAAUUGCCGAUAAGUAUGUCCGCA |
| | | AUUUACAACACAGACUUUAUGAGUGUCUCU |
| | | AUAGAAAUAGAGAUGUUGACACAGACUUUG |
| | | UGAAUGAGUUUUACGCAUAUUUGCGUAAAC |
| | | AUUUCUCAAUGAUGAUACUCUCUGACGAUG |
| | | CUGUUGUGUGUUUCAAUAGCACUUAUGCAU |
| | | CUCAAGGUCUAGUGGCUAGCAUAAAGAACU |
| | | UUAAGUCAGUUCUUUAUUAUCAAAACAAUG |
| | | UUUUUAUGUCUGAAGCAAAAUGUUGGACUG |
| | | AGACUGACCUUACUAAAGGACCUCAUGAAU |
| | | UUUGCUCUCAACAUACAAUGCUAGUUAAAC |
| | | AGGGUGAUGAUUAUGUGUACCUUCCUUACC |
| | | CAGAUCCAUCAAGAAUCCUAGGGGCCGGCU |
| | | GUUUUGUAGAUGAUAUCGUAAAAACAGAUG |
| | | GUACACUUAUGAUUGAACGGUUCGUGUCUU |
| | | UAGCUAUAGAUGCUUACCCACUUACUAAAC |
| | | AUCCUAAUCAGGAGUAUGCUGAUGUCUUUC |
| | | AUUUGUACUUACAAUACAUAAGAAAGCUAC |
| | | AUGAUGAGUUAACAGGACACAUGUUAGACA |
| | | UGUAUUCUGUUAUGCUUACUAAUGAUAACA |
| | | CUUCAAGGUAUUGGGAACCUGAG |
| 100 | Spike 21655/22420 | GGGUUUCACACGUGGUGUUUAUUACCCUGA |
| | | CAAAGUUUUCAGAUCCUCAGUUUUACAUUC |
| | | AACUCAGGACUUGUUCUUACCUUUCUUUUC |
| | | CAAUGUUACUUGGUUCCAUGCUAUACAUGU |
| | | CUCUGGGACCAAUGGUACUAAGAGGUUUGA |
| | | UAACCCUGUCCUACCAUUUAAUGAUGGUGU |
| | | UUAUUUUGCUUCCACUGAGAAGUCUAACAU |
| | | AAUAAGAGGCUGGAUUUUUGGUACUACUUU |
| | | AGAUUCGAAGACCCAGUCCCUACUUAUUGU |
| | | UAAUAACGCUACUAAUGUUGUUAUUAAAGU |
| | | CUGUGAAUUUCAAUUUUGUAAUGAUCCAUU |
| | | UUUGGGUGUUUAUUACCACAAAAACAACAA |
| | | AAGUUGGAUGGAAAGUGAGUUCAGAGUUUA |
| | | UUCUAGUGCGAAUAAUUGCACUUUUGAAUA |
| | | UGUCUCUCAGCCUUUUCUUAUGGACCUUGA |
| | | AGGAAAACAGGGUAAUUUCAAAAAUCUUAG |
| | | GGAAUUUGUGUUUAAGAAUAUUGAUGGUUA |
| | | UUUUAAAAUAUAUUCUAAGCACACGCCUAU |
| | | UAAUUUAGUGCGUGAUCUCCCUCAGGGUUU |
| | | UUCGGCUUUAGAACCAUUGGUAGAUUUGCC |
| | | AAUAGGUAUUAACAUCACUAGGUUUCAAAC |
| | | UUUACUUGCUUUACAUAGAAGUUAUUUGAC |
| | | UCCUGGUGAUUCUUCUUCAGGUUGGACAGC |
| | | UGGUGCUGCAGCUUAUUAUGUGGGUUAUCU |
| | | UCAACCUAGGACUUUUCUAUUAAAAUAUAA |
| | | UGAAAAUGGAACCAUUACA |
| 101 | Spike 22420/23122 | GGGAUGCUGUAGACUGUGCACUUGACCCUC |
| | | UCUCAGAAACAAAGUGUACGUUGAAAUCCU |
| | | UCACUGUAGAAAAAGGAAUCUAUCAAACUU |
| | | CUAACUUUAGAGUCCAACCAACAGAAUCUA |
| | | UUGUUAGAUUUCCUAAUAUUACAAACUUGU |
| | | GCCCUUUUGGUGAAGUUUUUAACGCCACCA |
| | | GAUUUGCAUCUGUUUAUGCUUGGAACAGGA |
| | | AGAGAAUCAGCAACUGUGUUGCUGAUUAUU |
| | | CUGUCCUAUAUAAUUCCGCAUCAUUUUCCA |
| | | CUUUUAAGUGUUAUGGAGUGUCUCCUACUA |
| | | AAUUAAAUGAUCUCUGCUUUACUAAUGUCU |
| | | AUGCAGAUUCAUUUGUAAUUAGAGGUGAUG |
| | | AAGUCAGACAAAUCGCUCCAGGGCAAACUG |
| | | GAAAGAUUGCUGAUUAUAAUUAUAAAUUAC |
| | | CAGAUGAUUUUACAGGCUGCGUUAUAGCUU |
| | | GGAAUUCUAACAAUCUUGAUUCUAAGGUUG |
| | | GUGGUAAUUAUAAUUACCUGUAUAGAUUGU |
| | | UUAGGAAGUCUAAUCUCAAACCUUUUGAGA |
| | | GAGAUAUUUCAACUGAAAUCUAUCAGGCCG |
| | | GUAGCACACCUUGUAAUGGUGUUGAAGGUU |
| | | UUAAUUGUUACUUUCCUUUACAAUCAUAUG |
| | | GUUUCCAACCCACUAAUGGUGUUGGUUACC |
| | | AACCAUACAGAGUAGUAGUACUUUCUUUUG |
| | | AACUUCUACAUGCA |
| 102 | Spike 23436/23911 | GGGUGCAGAUCAACUUACUCCUACUUGGCG |
| | | UGUUUAUUCUACAGGUUCUAAUGUUUUUCA |
| | | AACACGUGCAGGCUGUUUAAUAGGGGCUGA |
| | | ACAUGUCAACAACUCAUAUGAGUGUGACAU |
| | | ACCCAUUGGUGCAGGUAUAUGCGCUAGUUA |
| | | UCAGACUCAGACUAAUUCUCCUCGGCGGGC |
| | | ACGUAGUGUAGCUAGUCAAUCCAUCAUUGC |
| | | CUACACUAUGUCACUUGGUGCAGAAAAUUC |
| | | AGUUGCUUACUCUAAUAACUCUAUUGCCAU |
| | | ACCCACAAAUUUUACUAUUAGUGUUACCAC |
| | | AGAAAUUCUACCAGUGUCUAUGACCAAGAC |
| | | AUCAGUAGAUUGUACAAUGUACAUUUGUGG |
| | | UGAUUCAACUGAAUGCAGCAAUCUUUUGUU |
| | | GCAAUAUGGCAGUUUUUGUACACAAUUAAA |
| | | CCGUGCUUUAACUGGAAUAGCUGUUGAACA |
| | | AGACAAAAACACCCAAGAAGUUUUUGCA |
| 103 | Spike 24108/24665 | GGGUUUCCCCAUUUGUGCACAAAAGUUUAA |
| | | CGGCCUUACUGUUUUGCCACCUUUGCUCAC |
| | | AGAUGAAAUGAUUGCUCAAUACACUUCUGC |
| | | ACUGUUAGCGGGUACAAUCACUUCUGGUUG |
| | | GACCUUUGGUGCAGGUGCUGCAUUACAAAU |
| | | ACCAUUUGCUAUGCAAAUGGCUUAUAGGUU |
| | | UAAUGGUAUUGGAGUUACACAGAAUGUUCU |
| | | CUAUGAGAACCAAAAAUUGAUUGCCAACCA |
| | | AUUUAAUAGUGCUAUUGGCAAAAUUCAAGA |
| | | CUCACUUUCUUCCACAGCAAGUGCACUUGG |
| | | AAAACUUCAAGAUGUGGUCAACCAAAAUGC |
| | | ACAAGCUUUAAACACGCUUGUUAAACAACU |
| | | UAGCUCCAAUUUUGGUGCAAUUUCAAGUGU |
| | | UUUAAAUGAUAUCCUUUCACGUCUUGACAA |
| | | AGUUGAGGCUGAAGUGCAAAUUGAUAGGUU |
| | | GAUCACAGGCAGACUUCAAAGUUUGCAGAC |
| | | AUAUGUGACUCAACAAUUAAUUAGAGCUGC |
| | | AGAAAUCAGAGCUUCUGCUAAUCUUGCUGC |
| | | UACUAAAAUGUCAGAGUGUGUACUUG |
| 104 | Spike 24669/25343 | GGGCAAAAAUCAAAAAGAGUUGAUUUUUGU |
| | | GGAAAGGGCUAUCAUCUUAUGUCCUUCCCU |
| | | CAGUCAGCACCUCAUGGUGUAGUCUUCUUG |
| | | CAUGUGACUUAUGUCCCUGCACAAGAAAAG |
| | | AACUUCACAACUGCUCCUGCCAUUUGUCAU |
| | | GAUGGAAAAGCACACUUUCCUCGUGAAGGU |
| | | GUCUUUGUUUCAAAUGGCACACACUGGUUU |
| | | GUAACACAAAGGAAUUUUUAUGAACCACAA |
| | | AUCAUUACUACAGACAACACAUUUGUGUCU |
| | | GGUAACUGUGAUGUUGUAAUAGGAAUUGUC |
| | | AACAACACAGUUUAUGAUCCUUUGCAACCU |
| | | GAAUUAGACUCAUUCAAGGAGGAGUUAGAU |
| | | AAAUAUUUUAAGAAUCAUACAUCACCAGAU |
| | | GUUGAUUUAGGUGACAUCUCUGGCAUUAAU |
| | | GCUUCAGUUGUAAACAUUCAAAAAGAAAUU |
| | | GACCGCCUCAAUGAGGUUGCCAAGAAUUUA |
| | | AAUGAAUCUCUCAUCGAUCUCCAAGAACUU |
| | | GGAAAGUAUGAGCAGUAUAUAAAAUGGCCA |
| | | UGGUACAUUUGGCUAGGUUUUAUAGCUGGC |
| | | UUGAUUGCCAUAGUAAUGGUGACAAUUAUG |
| | | CUUUGCUGUAUGACCAGUUGCUGUAGUUGU |
| | | CUCAAGGGCUGUUGUUCUUGUGGAUCCUGC |
| | | UGCAAAUUUGAUGAAGACGACU |
| 105 | dZ_10098a | TACTTGTACCATACAAGGCTAGCTACAACG |
| | | ACCTCAACT |
| 106 | dZ_10140a | GTCATCAAGCCAAAGAGGCTAGCTACAACG |
| | | ACGTTAAGT |
| 107 | dZ_10176a | AGAGGTGCAGATCACAGGCTAGCTACAACG |
| | | AGTCTTGGA |
| 108 | dZ_10256a | ACATTACCAGCCTGTAGGCTAGCTACAACG |
| | | ACAAGAAAT |
| 109 | dZ_10325a | GGATTGGCTGTATCAAGGCTAGCTACAACG |
| | | ACTTAAGCT |
| 110 | dZ_10338a | CTTAGGTGTCTTAGGAGGCTAGCTACAACG |
| | | ATGGCTGTA |
| 111 | dZ_10442a | GTGAAATTGGGCCTCAGGCTAGCTACAACG |
| | | AAGCACATT |
| 112 | dZ_10491a | GTTAAAACCAACACTAGGCTAGCTACAACG |
| | | ACACATGAA |
| 113 | dZ_10599a | GTCAACAAAAGGTCCAGGCTAGCTACAACG |
| | | AAAAAGTTA |
| 114 | dZ_10800a | GAAAGAGGTCCTAGTAGGCTAGCTACAACG |
| | | AGTCAACAT |
| 115 | dZ_11062a | AAAAGAACAAAGACCAGGCTAGCTACAACG |
| | | ATGAGTACT |
| 116 | dZ_11085a | TAAAAAGGCATTTTCAGGCTAGCTACAACG |
| | | AACAAAAAA |
| 117 | dZ_11111a | ATAGCAATAATACCCAGGCTAGCTACAACG |
| | | AAGCAAAAG |
| 118 | dZ_11217a | AGGCATATAGACCATAGGCTAGCTACAACG |
| | | ATAAAATAA |
| 119 | dZ_11270a | AAACTAGTATCAACCAGGCTAGCTACAACG |
| | | AATCCAACC |
| 120 | dZ_11342a | CTTGCTGTCATAAGGAGGCTAGCTACAACG |
| | | ATAGTAACA |
| 121 | dZ_11502a | GACAGTTGTAACTACAGGCTAGCTACAACG |
| | | ACTGAGTAG |
| 122 | dZ_11521a | TACCTCTGGCCAAAAAGGCTAGCTACAACG |
| | | AATGACAGT |
| 123 | dZ_11567a | CCAGTTATGAAGAAAAGGCTAGCTACAACG |
| | | AAGGGCAAT |
| 124 | dZ_11616a | AAAATAGCCTAAGAAAGGCTAGCTACAACG |
| | | AAATAAACT |
| 125 | dZ_11697a | AGAAACTAAGTAATCAGGCTAGCTACAACG |
| | | AAAACACCA |
| 126 | dZ_11730a | TCCCTGTGAATTCATAGGCTAGCTACAACG |
| | | AATCTAAAC |
| 127 | dZ_12156a | AGCAACAGCCTGCTCAGGCTAGCTACAACG |
| | | AAAGCTTCT |
| 128 | dZ_12174a | AACTTCAGAATCACCAGGCTAGCTACAACG |
| | | ATAGCAACA |
| 129 | dZ_12202a | TCAAAGACTTCTTCAAGGCTAGCTACAACG |
| | | ATTTTTAAG |
| 130 | dZ_12262a | CATCTTTTCCAACTTAGGCTAGCTACAACG |
| | | AGTTGCATG |
| 131 | dZ_12290a | TTATACATTTGGGTCAGGCTAGCTACAACG |
| | | AAGCTTGAT |
| 132 | dZ_12299a | CTAGCCTGTTTATACAGGCTAGCTACAACG |
| | | ATTGGGTCA |
| 133 | dZ_12350a | AAAAGCATTGTCTGCAGGCTAGCTACAACG |
| | | AAGCACTAG |
| 134 | dZ_12359a | AGCATAGTGAAAAGCAGGCTAGCTACAACG |
| | | ATGTCTGCA |
| 135 | dZ_12495a | ATTTTTATATGTGTTAGGCTAGCTACAACG |
| | | AAGTCTGGT |
| 136 | dZ_12557a | TCTACAACCTGTTGGAGGCTAGCTACAACG |
| | | ATTCCCACA |
| 137 | dZ_12618a | TGCTAAATTAGGTGAAGGCTAGCTACAACG |
| | | ATGTCCATA |
| 138 | dZ_19699a | TAAACAGTGTTATTAAGGCTAGCTACAACG |
| | | AGATAGAAA |
| 139 | dZ_19743a | TTTTATTTTCAAACAAGGCTAGCTACAACG |
| | | ATCTACATC |
| 140 | dZ_19825a | TTATTGAGTATTTTCAGGCTAGCTACAACG |
| | | ACTCTGGTA |
| 141 | dZ_19892a | TATATGTGCTGGAGCAGGCTAGCTACAACG |
| | | ACTCTTTTG |
| 142 | dZ_19915a | ATAGAACAAACACCAAGGCTAGCTACAACG |
| | | AAGTAGATA |
| 143 | dZ_19963a | GTGAGTGGTGCACAAAGGCTAGCTACAACG |
| | | ACGTTTCAG |
| 144 | dZ_20103a | TGTGACTCCATTAAGAGGCTAGCTACAACG |
| | | ATAGCTTGT |
| 145 | dZ_20134a | TTGAACTGTGTTTTTAGGCTAGCTACAACG |
| | | AGGCTTCTC |
| 146 | dZ_20156a | ACCATCAACTTTCTTAGGCTAGCTACAACG |
| | | AAATAATTG |
| 147 | dZ_20184a | AGTAAGTTTCAGGTAAGGCTAGCTACAACG |
| | | ATGTTGGAC |
| 148 | dZ_20216a | TTTAAATTCTTGTAAAGGCTAGCTACAACG |
| | | ATTCTACTC |
| 149 | dZ_20251a | AATTCTAAGAAATCAAGGCTAGCTACAACG |
| | | ATTCCATTT |
| 150 | dZ_20276a | CCGTTCAATGAATTCAGGCTAGCTACAACG |
| | | ACCATAGCT |
| 151 | dZ_20412a | GAATAAAATCTTCTAAGGCTAGCTACAACG |
| | | ATCAAAAGG |
| 152 | dZ_20426a | GTACTGTCCATAGGAAGGCTAGCTACAACG |
| | | AAAAATCTT |
| 153 | dZ_20511a | CAAAATCATCAAGTAAGGCTAGCTACAACG |
| | | AAAATCAAT |
| 154 | dZ_16334a | TGATGTTGATATGACAGGCTAGCTACAACG |
| | | AGGTCGTAA |
| 155 | dZ_16485a | CTTGTCCATTAGCACAGGCTAGCTACAACG |
| | | AAATGGAAA |
| 156 | dZ_16501a | TTATATAAACCAAAAAGGCTAGCTACAACG |
| | | ATTGTCCAT |
| 157 | dZ_16583a | AATGTAATCACCAGCAGGCTAGCTACAACG |
| | | ATTGTCCAG |
| 158 | dZ_16727a | AACTTCCCATGAAAGAGGCTAGCTACAACG |
| | | AGTAATTCT |
| 159 | dZ_16890a | AATCACCAACATTTAAGGCTAGCTACAACG |
| | | ATTGTAAGT |
| 160 | dZ_16912a | GTATGTGATGTCAGCAGGCTAGCTACAACG |
| | | AAAAATAAT |
| 161 | dZ_16925a | TAATGGCATTACTGTAGGCTAGCTACAACG |
| | | AGTGATGTC |
| 162 | dZ_16981a | GGGTATAAGCCAGTAAGGCTAGCTACAACG |
| | | ATCTAACAT |
| 163 | dZ_17207a | TTTATCTATAGGCAAAGGCTAGCTACAACG |
| | | AATTTTAAT |
| 164 | dZ_17344a | TCATCAAAGACAACTAGGCTAGCTACAACG |
| | | AATCTGCTG |
| 165 | dZ_17378a | AACACTCAAATCATAAGGCTAGCTACAACG |
| | | ATTGTGGCC |
| 166 | dZ_17406a | AGTGCTTAGCACGTAAGGCTAGCTACAACG |
| | | ACTGGCATT |
| 167 | dZ_17498a | ACACACTGAATTGAAAGGCTAGCTACAACG |
| | | AATTCTGGT |
| 168 | dZ_17522a | GGACCTATAGTTTTCAGGCTAGCTACAACG |
| | | AAAGTCTAC |
| 169 | dZ_17567a | AACAATTTCAGCAGGAGGCTAGCTACAACG |
| | | AAACGCCGA |
| 170 | dZ_17658a | TAACACCCTTATAAAAGGCTAGCTACAACG |
| | | AATTTTAAA |
| 171 | dZ_17713a | TCTCTTACCACGCCTAGGCTAGCTACAACG |
| | | ATTGTGGCC |
| 172 | dZ_17730a | GGTTACGTGTAAGGAAGGCTAGCTACAACG |
| | | ATCTCTTAC |
| 173 | dZ_17780a | AGCATTCTGTGAATTAGGCTAGCTACAACG |
| | | AAAGGTGAA |
| 174 | dZ_18135a | AACCTTCAGTTTTGAAGGCTAGCTACAACG |
| | | ATTAGTGTC |
| 175 | dZ_18153a | CAGGTATGTCAACACAGGCTAGCTACAACG |
| | | AAAACCTTC |
| 176 | dZ_18235a | TTAGGGTAACCATTAAGGCTAGCTACAACG |
| | | ATTGATAAT |
| 177 | dZ_18259a | GCTTCTTCGCGGGTGAGGCTAGCTACAACG |
| | | AAAACATGT |
| 178 | dZ_18391a | CCTGTAGGTACAGCAAGGCTAGCTACAACG |
| | | ATAGGTTAA |
| 179 | dZ_18470a | GAGGTGTTTAAATTGAGGCTAGCTACAACG |
| | | ACTCCAGGC |
| 180 | dZ_18498a | AAGGAAGTCCTTTGTAGGCTAGCTACAACG |
| | | AATAAGTGG |
| 181 | dZ_18535a | CTTAACATTTGTACAAGGCTAGCTACAACG |
| | | ACTTTATAC |
| 182 | dZ_18583a | GCCCATAAGACAAATAGGCTAGCTACAACG |
| | | AGACTCTGT |
| 183 | dZ_18640a | GTGCGCTCAGGTCCTAGGCTAGCTACAACG |
| | | ATTTCACAA |
| 184 | dZ_18791a | GTTGCTTTGTAGGTTAGGCTAGCTACAACG |
| | | ACTGTAAAA |
| 185 | dZ_18818a | ACCATGGACTTGACAAGGCTAGCTACAACG |
| | | AACAGATCA |
| 186 | dZ_18919a | ATTATAGGATATTCAAGGCTAGCTACAACG |
| | | AAGTCCAGT |
| 187 | dZ_18941a | ATTAATCTTCAGTTCAGGCTAGCTACAACG |
| | | ACACCAATT |
| 188 | dZ_18973a | ACAACCATGTGTTGAAGGCTAGCTACAACG |
| | | ACTTTCTAC |
| 189 | dZ_19033a | GCTTTAGGGTTACCAAGGCTAGCTACAACG |
| | | AGTCGTGAA |
| 190 | dZ_19182a | AATTCCAAAATAGGCAGGCTAGCTACAACG |
| | | AACACCATC |
| 191 | dZ_19334a | AACAAAAGCACTTTTAGGCTAGCTACAACG |
| | | ACAAAAGCT |
| 192 | dZ_19376a | TGGACTGTCAGAGTAAGGCTAGCTACAACG |
| | | AAGAAAAAT |
| 193 | dZ_19398a | CTTGTTTTCCATGAGAGGCTAGCTACAACG |
| | | ATCACATGG |
| 194 | dZ_15501a | GGGAGTGAGGCTTGTAGGCTAGCTACAACG |
| | | ACGGTATCG |
| 195 | dZ_25524a | CGCCAACAATAAGCCAGGCTAGCTACAACG |
| | | ACCGAAAGG |
| 196 | dZ_25540a | ACAGCAAGAAGTGCAAGGCTAGCTACAACG |
| | | AGCCAACAA |
| 197 | dZ_25556a | GAAGCGCTCTGAAAAAGGCTAGCTACAACG |
| | | AAGCAAGAA |
| 198 | dZ_25596a | AGAGTGCTAGTTGCCAGGCTAGCTACAACG |
| | | ACTCTTTTT |
| 199 | dZ_25621a | TTGCAAACAAAGTGAAGGCTAGCTACAACG |
| | | AACCCTTGG |
| 200 | dZ_25647a | AAACTGTTACAAACAAGGCTAGCTACAACG |
| | | AAACAGCAA |
| 201 | dZ_25660a | AAAAGGTGTGAGTAAAGGCTAGCTACAACG |
| | | ATGTTACAA |
| 202 | dZ_25765a | CAAAGCCAAAGCCTCAGGCTAGCTACAACG |
| | | ATATTATTC |
| 203 | dZ_25806a | TGGCATCATAAAGTAAGGCTAGCTACAACG |
| | | AGGGTTTTT |
| 204 | dZ_25826a | ATGCCAGCAAAGAAAAGGCTAGCTACAACG |
| | | AAGTTGGCA |
| 205 | dZ_25847a | ACAATAGTCGTAACAAGGCTAGCTACAACG |
| | | ATAGTATGC |
| 206 | dZ_25937a | ACCAATCTGGTAGTCAGGCTAGCTACAACG |
| | | AGTTCAGAA |
| 207 | dZ_25967a | TTACTCCAGATTCCCAGGCTAGCTACAACG |
| | | ATTTTCAGT |
| 208 | dZ_26072a | GATGAAGAAGGTAACAGGCTAGCTACAACG |
| | | AGTTCAACA |
| 209 | dZ_26155a | ATTACTGGATTAACAAGGCTAGCTACAACG |
| | | ATCCGGATG |
| 210 | dZ_341a | AAGCCACGTACGAGCAGGCTAGCTACAACG |
| | | AGTCGCGAA |
| 211 | dZ_355a | CGTGCCTCTGATAAGAGGCTAGCTACAACG |
| | | ACTCCTCCA |
| 212 | dZ_426a | CCTTTTTCAACTTCTAGGCTAGCTACAACG |
| | | ATAAGCCAC |
| 213 | dZ_468a | ACGTTTGATGAACACAGGCTAGCTACAACG |
| | | AAGGGCTGT |
| 214 | dZ_483a | AGTTCGAGCATCCGAAGGCTAGCTACAACG |
| | | AGTTTGATG |
| 215 | dZ_507a | AACCATAACATGACCAGGCTAGCTACAACG |
| | | AGAGGTGCA |
| 216 | dZ_558a | TGTCTCACCACTACGAGGCTAGCTACAACG |
| | | ACGTACTGA |
| 217 | dZ_578a | ATGAGGGACAAGGACAGGCTAGCTACAACG |
| | | ACAAGTGTC |
| 218 | dZ_648a | GCCACCAGCTCCTTTAGGCTAGCTACAACG |
| | | ATACCGTTC |
| 219 | dZ_688a | CGCCTAAGTCAAATGAGGCTAGCTACAACG |
| | | ATTTAGATC |
| 220 | dZ_765a | TTCACGGGTAACACCAGGCTAGCTACAACG |
| | | ATGCTATGT |
| 221 | dZ_20716a | CACTTTTCTAATAGCAGGCTAGCTACAACG |
| | | ATCTTTGCA |
| 222 | dZ_20730a | AATTTTGAAGGTCACAGGCTAGCTACAACG |
| | | ATTTTCTAA |
| 223 | dZ_20756a | TTTAGGTAATGTTGCAGGCTAGCTACAACG |
| | | ATATCACCA |
| 224 | dZ_20788a | TGAGTATATTTTGCGAGGCTAGCTACAACG |
| | | AATTCATCA |
| 225 | dZ_20817a | TGTTAATGTGTTTAAAGGCTAGCTACAACG |
| | | AATTGACAC |
| 226 | dZ_20851a | AAATGTATAACTCTCAGGCTAGCTACAACG |
| | | AATTATAGG |
| 227 | dZ_20882a | TGGTGCAACTCCTTTAGGCTAGCTACAACG |
| | | ACAGAACCA |
| 228 | dZ_20954a | GACAAAGTCATTAAGAGGCTAGCTACAACG |
| | | ACTGAATCG |
| 229 | dZ_20992a | GTTGCACAATCACCAAGGCTAGCTACAACG |
| | | ACAAAGTTG |
| 230 | dZ_21086a | ACCCTCTTTAGAGTCAGGCTAGCTACAACG |
| | | ATTTCTTTT |
| 231 | dZ_21127a | GCTAGCTTTTGTTGTAGGCTAGCTACAACG |
| | | AAAACCCAC |
| 232 | dZ_21115a | TGTATAAACCCACAAAGGCTAGCTACAACG |
| | | AGTAAGTGA |
| 233 | dZ_21238a | GCATTCACATTAGTAAGGCTAGCTACAACG |
| | | AAAAGGCTG |
| 234 | dZ_21290a | GCGTGGTTTGCCAAGAGGCTAGCTACAACG |
| | | AAATTACAT |
| 235 | dZ_21313a | ATGACATAACCATCTAGGCTAGCTACAACG |
| | | ATTGTTCGC |
| 236 | dZ_21338a | CCTCCAAAATATGTAAGGCTAGCTACAACG |
| | | ATTGCATGC |
| 237 | dZ_21345a | TTGTATTCCTCCAAAAGGCTAGCTACAACG |
| | | AATGTAATT |
| 238 | dZ_21390a | ATTTACTCATGTCAAAGGCTAGCTACAACG |
| | | AAAAGAATA |
| 239 | dZ_21467a | AGAAGAGATAAAATCAGGCTAGCTACAACG |
| | | AATCATTGA |
| 240 | dZ_846a | CTCAAGAGGGTAGCCAGGCTAGCTACAACG |
| | | ACAGGGCCA |
| 241 | dZ_866a | GCTAGAAGGTCTTTAAGGCTAGCTACAACG |
| | | AGCACTCAA |
| 242 | dZ_910a | AGTCCAGTTGTTCGGAGGCTAGCTACAACG |
| | | AAAAGTGCA |
| 243 | dZ_1015a | CAAAAGGTGTCTGCAAGGCTAGCTACAACG |
| | | ATCATAGCT |
| 244 | dZ_1051a | CATTGAAGGTGTCAAAGGCTAGCTACAACG |
| | | ATTCTTTGC |
| 245 | dZ_1080a | TAAGGGAAATACAAAAGGCTAGCTACAACG |
| | | ATTGGACAT |
| 246 | dZ_1168a | CAACTGGATAGACAGAGGCTAGCTACAACG |
| | | ACGAATTCT |
| 247 | dZ_1210a | TGAGAGTTGAAAGGCAGGCTAGCTACAACG |
| | | AATTTGGTT |
| 248 | dZ_1243a | CCATGAAGTTTCACCAGGCTAGCTACAACG |
| | | AAATGATCA |
| 249 | dZ_1308a | ACCTTCTTTAGTCAAAGGCTAGCTACAACG |
| | | ATCTCAGTG |
| 250 | dZ_1338a | ATTTTGGGGTAAGTAAGGCTAGCTACAACG |
| | | ACACAAGTA |
| 251 | dZ_1367a | CATGCTGGACAATAAAGGCTAGCTACAACG |
| | | ATTTAACAA |
| 252 | dZ_1431a | TTTCAAGCCAGATTCAGGCTAGCTACAACG |
| | | ATATGGTAT |
| 253 | dZ_1475a | CAGCCTCCAAAGGCAAGGCTAGCTACAACG |
| | | AAGTGCGAC |
| 254 | dZ_1599a | AAGGTTGTCATTAAGAGGCTAGCTACAACG |
| | | ACTTCGGAA |
| 255 | dZ_1719a | AGTTTCCACAAAAGCAGGCTAGCTACAACG |
| | | ATTGTGGAA |
| 256 | dZ_1759a | CAACAATTTGTTTGAAGGCTAGCTACAACG |
| | | AGCTTTATA |
| 257 | dZ_1796a | GCTTTTCCTTTTGTAAGGCTAGCTACAACG |
| | | ATTTAAAAT |
| 258 | dZ_1846a | GAGGACTCAGTATTGAGGCTAGCTACAACG |
| | | ATTCTGTTC |
| 259 | dZ_1940a | TTCTGTAAAACACGCAGGCTAGCTACAACG |
| | | AAGAATTTT |
| 260 | dZ_2020a | CCAAATCAGATGTGAAGGCTAGCTACAACG |
| | | AATCATAGC |
| 261 | dZ_2127a | GGGTTTGAGTTTTTCAGGCTAGCTACAACG |
| | | AAAACAGTG |
| 262 | dZ_2167a | CTACACCTTCCTTAAAGGCTAGCTACAACG |
| | | ATTCTCTTC |
| 263 | dZ_2244a | ACAATTTGTCCACCGAGGCTAGCTACAACG |
| | | AAATTTCAC |
| 264 | dZ_2276a | TGAACACTCTCCTTAAGGCTAGCTACAACG |
| | | ATTCCTTTG |
| 265 | dZ_2376a | AAATGTTTCACCTAAAGGCTAGCTACAACG |
| | | ATCAAGGCT |
| 266 | dZ_2426a | TCTTCTCTGGATTTAAGGCTAGCTACAACG |
| | | AACACTTTC |
| 267 | dZ_3030a | TCTTCTCTGGATTTAAGGCTAGCTACAACG |
| | | AACACTTTC |
| 268 | dZ_3072a | AAACTCTTCTTCTTCAGGCTAGCTACAACG |
| | | AAATCACCT |
| 269 | dZ_3124a | TTTACCTTGGTAATCAGGCTAGCTACAACG |
| | | ACTTCAGTA |
| 270 | dZ_3207a | TTGTTGACTATCATCAGGCTAGCTACAACG |
| | | ACTAACCAA |
| 271 | dZ_3377a | GCATTTTTAATGTATAGGCTAGCTACAACG |
| | | AATTGTCAG |
| 272 | dZ_3419a | ACCACTGTTGGTTTTAGGCTAGCTACAACG |
| | | ACTTTTTAG |
| 273 | dZ_3512a | TCAGATTCAACTTGCAGGCTAGCTACAACG |
| | | AGGCATTGT |
| 274 | dZ_3531a | ATTAGTAGCTATGTAAGGCTAGCTACAACG |
| | | ACATCAGAT |
| 275 | dZ_3647a | CTCTTAAGAAGTTGAAGGCTAGCTACAACG |
| | | AGTCTTCAC |
| 276 | dZ_3681a | TAGAACTTCGTGCTGAGGCTAGCTACAACG |
| | | ATAAAATTT |
| 277 | dZ_3706a | TACCAGCTGATAATAAGGCTAGCTACAACG |
| | | AGGTGCAAG |
| 278 | dZ_3755a | ACAGTATCTACACAAAGGCTAGCTACAACG |
| | | ATCTTAAAG |
| 279 | dZ_3782a | AAGACAGCTAAGTAGAGGCTAGCTACAACG |
| | | AATTTGTGC |
| 280 | dZ_3813a | TGAAACAAGTTTGTCAGGCTAGCTACAACG |
| | | AAGAGATTT |
| 281 | dZ_3908a | GGTTTACTTTCAGTTAGGCTAGCTACAACG |
| | | AAAATGGCT |
| 282 | dZ_3960a | TCAACACAAGCTTTGAGGCTAGCTACAACG |
| | | ATTTCTTAT |
| 283 | dZ_4044a | TGGATGAAGATTGCCAGGCTAGCTACAACG |
| | | ATAATGTCA |
| 284 | dZ_4076a | ATGTCAATGTCACTAAGGCTAGCTACAACG |
| | | AAAGAGTGG |
| 285 | dZ_4118a | CATCACCCACTATATAGGCTAGCTACAACG |
| | | AGGAGCATC |
| 286 | dZ_4148a | ACCACAGCAGTTAAAAGGCTAGCTACAACG |
| | | AACCCTCTT |
| 287 | dZ_4239a | CGGGTAAGTGGTTATAGGCTAGCTACAACG |
| | | AAATTGTCT |
| 288 | dZ_4269a | CTCTACAGTGTAACCAGGCTAGCTACAACG |
| | | ATTAAACCC |
| 289 | dZ_4298a | TTACACTTTTTAAGCAGGCTAGCTACAACG |
| | | ATGTCTTTG |
| 290 | dZ_4317a | TAGAATGTAAAAGGCAGGCTAGCTACAACG |
| | | ATTTTACAC |
| 291 | dZ_4343a | TGCTTCTCATTAGAGAGGCTAGCTACAACG |
| | | AAATAGATG |
| 292 | dZ_4386a | AAGCATTTCTCGCAAAGGCTAGCTACAACG |
| | | ATCCAAGAA |
| 293 | dZ_4528a | TGGTGTAAAAGTAAAAGGCTAGCTACAACG |
| | | ACTAGCACC |
| 294 | dZ_4590a | TGTAACAAGAGTTTCAGGCTAGCTACAACG |
| | | ATTAGATCG |
| 295 | dZ_4731a | AGAAGAAGAAGTAAGAGGCTAGCTACAACG |
| | | AAACCATTA |
| 296 | Membrane 26523/27192 | GGGATGGCAGATTCCAACGGTACTATTACC |
| | | GTTGAAGAGCTTAAAAAGCTCCTTGAACAA |
| | | TGGAACCTAGTAATAGGTTTCCTATTCCTT |
| | | ACATGGATTTGTCTTCTACAATTTGCCTAT |
| | | GCCAACAGGAATAGGTTTTTGTATATAATT |
| | | AAGTTAATTTTCCTCTGGCTGTTATGGCCA |
| | | GTAACTTTAGCTTGTTTTGTGCTTGCTGCT |
| | | GTTTACAGAATAAATTGGATCACCGGTGGA |
| | | ATTGCTATCGCAATGGCTTGTCTTGTAGGC |
| | | TTGATGTGGCTCAGCTACTTCATTGCTTCT |
| | | TTCAGACTGTTTGCGCGTACGCGTTCCATG |
| | | TGGTCATTCAATCCAGAAACTAACATTCTT |
| | | CTCAACGTGCCACTCCATGGCACTATTCTG |
| | | ACCAGACCGCTTCTAGAAAGTGAACTCGTA |
| | | ATCGGAGCTGTGATCCTTCGTGGACATCTT |
| | | CGTATTGCTGGACACCATCTAGGACGCTGT |
| | | GACATCAAGGACCTGCCTAAAGAAATCACT |
| | | GTTGCTACATCACGAACGCTTTCTTATTAC |
| | | AAATTGGGAGCTTCGCAGCGTGTAGCAGGT |
| | | GACTCAGGTTTTGCTGCATACAGTCGCTAC |
| | | AGGATTGGCAACTATAAATTAAACACAGAC |
| | | CATTCCAGTAGCAGTGACAATATTGCTTTG |
| | | CTTGTACAGTAAG |
| 297 | 3CL 10054/10972 | GGGAGTGGTTTTAGAAAAATGGCATTCCCA |
| | | TCTGGTAAAGTTGAGGGTTGTATGGTACAA |
| | | GTAACTTGTGGTACAACTACACTTAACGGT |
| | | CTTTGGCTTGATGACGTAGTTTACTGTCCA |
| | | AGACATGTGATCTGCACCTCTGAAGACATG |
| | | CTTAACCCTAATTATGAAGATTTACTCATT |
| | | CGTAAGTCTAATCATAATTTCTTGGTACAG |
| | | GCTGGTAATGTTCAACTCAGGGTTATTGGA |
| | | CATTCTATGCAAAATTGTGTACTTAAGCTT |
| | | AAGGTTGATACAGCCAATCCTAAGACACCT |
| | | AAGTATAAGTTTGTTCGCATTCAACCAGGA |
| | | CAGACTTTTTCAGTGTTAGCTTGTTACAAT |
| | | GGTTCACCATCTGGTGTTTACCAATGTGCT |
| | | ATGAGGCCCAATTTCACTATTAAGGGTTCA |
| | | TTCCTTAATGGTTCATGTGGTAGTGTTGGT |
| | | TTTAACATAGATTATGACTGTGTCTCTTTT |
| | | TGTTACATGCACCATATGGAATTACCAACT |
| | | GGAGTTCATGCTGGCACAGACTTAGAAGGT |
| | | AACTTTTATGGACCTTTTGTTGACAGGCAA |
| | | ACAGCACAAGCAGCTGGTACGGACACAACT |
| | | ATTACAGTTAATGTTTTAGCTTGGTTGTAC |
| | | GCTGCTGTTATAAATGGAGACAGGTGGTTT |
| | | CTCAATCGATTTACCACAACTCTTAATGAC |
| | | TTTAACCTTGTGGCTATGAAGTACAATTAT |
| | | GAACCTCTAACACAAGACCATGTTGACATA |
| | | CTAGGACCTCTTTCTGCTCAAACTGGAATT |
| | | GCCGTTTTAGATATGTGTGCTTCATTAAAA |
| | | GAATTACTGCAAAATGGTATGAATGGACGT |
| | | ACCATATTGGGTAGTGCTTTATTAGAAGAT |
| | | GAATTTACACCTTTTGATGTTGTTAGACAA |
| | | TGCTCAGGTGTTACTTTCCAA |
| 298 | NSP6 10992/11832 | GGGTCAAGGGTACACACCACTGGTTGTTAC |
| | | TCACAATTTTGACTTCACTTTTAGTTTTAG |
| | | TCCAGAGTACTCAATGGTCTTTGTTCTTTT |
| | | TTTTGTATGAAAATGCCTTTTTACCTTTTG |
| | | CTATGGGTATTATTGCTATGTCTGCTTTTG |
| | | CAATGATGTTTGTCAAACATAAGCATGCAT |
| | | TTCTCTGTTTGTTTTTGTTACCTTCTCTTG |
| | | CCACTGTAGCTTATTTTAATATGGTCTATA |
| | | TGCCTGCTAGTTGGGTGATGCGTATTATGA |
| | | CATGGTTGGATATGGTTGATACTAGTTTGT |
| | | CTGGTTTTAAGCTAAAAGACTGTGTTATGT |
| | | ATGCATCAGCTGTAGTGTTACTAATCCTTA |
| | | TGACAGCAAGAACTGTGTATGATGATGGTG |
| | | CTAGGAGAGTGTGGACACTTATGAATGTCT |
| | | TGACACTCGTTTATAAAGTTTATTATGGTA |
| | | ATGCTTTAGATCAAGCCATTTCCATGTGGG |
| | | CTCTTATAATCTCTGTTACTTCTAACTACT |
| | | CAGGTGTAGTTACAACTGTCATGTTTTTGG |
| | | CCAGAGGTATTGTTTTTATGTGTGTTGAGT |
| | | ATTGCCCTATTTTCTTCATAACTGGTAATA |
| | | CACTTCAGTGTATAATGCTAGTTTATTGTT |
| | | TCTTAGGCTATTTTTGTACTTGTTACTTTG |
| | | GCCTCTTTTGTTTACTCAACCGCTACTTTA |
| | | GACTGACTCTTGGTGTTTATGATTACTTAG |
| | | TTTCTACACAGGAGTTTAGATATATGAATT |
| | | CACAGGGACTACTCCCACCCAAGAATAGCA |
| | | TAGATGCCTTCAAACTCAACATTAAATTGT |
| | | TGGGTGTTGGTGGCAAACCTTGTATCAAAG |
| | | TAGC |
| 299 | NSP8 12098/12679 | GGGCCTCAGAGTTTAGTTCCCTTCCATCAT |
| | | ATGCAGCTTTTGCTACTGCTCAAGAAGCTT |
| | | ATGAGCAGGCTGTTGCTAATGGTGATTCTG |
| | | AAGTTGTTCTTAAAAAGTTGAAGAAGTCTT |
| | | TGAATGTGGCTAAATCTGAATTTGACCGTG |
| | | ATGCAGCCATGCAACGTAAGTTGGAAAAGA |
| | | TGGCTGATCAAGCTATGACCCAAATGTATA |
| | | AACAGGCTAGATCTGAGGACAAGAGGGCAA |
| | | AAGTTACTAGTGCTATGCAGACAATGCTTT |
| | | TCACTATGCTTAGAAAGTTGGATAATGATG |
| | | CACTCAACAACATTATCAACAATGCAAGAG |
| | | ATGGTTGTGTTCCCTTGAACATAATACCTC |
| | | TTACAACAGCAGCCAAACTAATGGTTGTCA |
| | | TACCAGACTATAACACATATAAAAATACGT |
| | | GTGATGGTACAACATTTACTTATGCATCAG |
| | | CATTGTGGGAAATCCAACAGGTTGTAGATG |
| | | CAGATAGTAAAATTGTTCAACTTAGTGAAA |
| | | TTAGTATGGACAATTCACCTAATTTAGCAT |
| | | GGCCTCTTATTGTAACAGCTTTAAGGGCCA |
| | | ATTCTGCTGTCAAA |
| 300 | NSP15 19620/20659 | GGGAGTTTAGAAAATGTGGCTTTTAATGTT |
| | | GTAAATAAGGGACACTTTGATGGACAACAG |
| | | GGTGAAGTACCAGTTTCTATCATTAATAAC |
| | | ACTGTTTACACAAAAGTTGATGGTGTTGAT |
| | | GTAGAATTGTTTGAAAATAAAACAACATTA |
| | | CCTGTTAATGTAGCATTTGAGCTTTGGGCT |
| | | AAGCGCAACATTAAACCAGTACCAGAGGTG |
| | | AAAATACTCAATAATTTGGGTGTGGACATT |
| | | GCTGCTAATACTGTGATCTGGGACTACAAA |
| | | AGAGATGCTCCAGCACATATATCTACTATT |
| | | GGTGTTTGTTCTATGACTGACATAGCCAAG |
| | | AAACCAACTGAAACGATTTGTGCACCACTC |
| | | ACTGTCTTTTTTGATGGTAGAGTTGATGGT |
| | | CAAGTAGACTTATTTAGAAATGCCCGTAAT |
| | | GGTGTTCTTATTACAGAAGGTAGTGTTAAA |
| | | GGTTTACAACCATCTGTAGGTCCCAAACAA |
| | | GCTAGTCTTAATGGAGTCACATTAATTGGA |
| | | GAAGCCGTAAAAACACAGTTCAATTATTAT |
| | | AAGAAAGTTGATGGTGTTGTCCAACAATTA |
| | | CCTGAAACTTACTTTACTCAGAGTAGAAAT |
| | | TTACAAGAATTTAAACCCAGGAGTCAAATG |
| | | GAAATTGATTTCTTAGAATTAGCTATGGAT |
| | | GAATTCATTGAACGGTATAAATTAGAAGGC |
| | | TATGCCTTCGAACATATCGTTTATGGAGAT |
| | | TTTAGTCATAGTCAGTTAGGTGGTTTACAT |
| | | CTACTGATTGGACTAGCTAAACGTTTTAAG |
| | | GAATCACCTTTTGAATTAGAAGATTTTATT |
| | | CCTATGGACAGTACAGTTAAAAACTATTTC |
| | | ATAACAGATGCGCAAACAGGTTCATCTAAG |
| | | TGTGTGTGTTCTGTTATTGATTTATTACTT |
| | | GATGATTTTGTTGAAATAATAAAATCCCAA |
| | | GATTTATCTGTAGTTTCTAAGGTTGTCAAA |
| | | GTGACTATTGACTATACAGAAATTTCATTT |
| | | ATGCTTTGGTGTAAAGATGGCCATGTAGAA |
| | | ACATTTTACCCAAAATTACAAT |
| 301 | Methyl-Transferase | GGGTCTAGTCAAGCGTGGCAACCGGGTGTT |
| | 20659/21545 | GCTATGCCTAATCTTTACAAAATGCAAAGA |
| | | ATGCTATTAGAAAAGTGTGACCTTCAAAAT |
| | | TATGGTGATAGTGCAACATTACCTAAAGGC |
| | | ATAATGATGAATGTCGCAAAATATACTCAA |
| | | CTGTGTCAATATTTAAACACATTAACATTA |
| | | GCTGTACCCTATAATATGAGAGTTATACAT |
| | | TTTGGTGCTGGTTCTGATAAAGGAGTTGCA |
| | | CCAGGTACAGCTGTTTTAAGACAGTGGTTG |
| | | CCTACGGGTACGCTGCTTGTCGATTCAGAT |
| | | CTTAATGACTTTGTCTCTGATGCAGATTCA |
| | | ACTTTGATTGGTGATTGTGCAACTGTACAT |
| | | ACAGCTAATAAATGGGATCTCATTATTAGT |
| | | GATATGTACGACCCTAAGACTAAAAATGTT |
| | | ACAAAAGAAAATGACTCTAAAGAGGGTTTT |
| | | TTCACTTACATTTGTGGGTTTATACAACAA |
| | | AAGCTAGCTCTTGGAGGTTCCGTGGCTATA |
| | | AAGATAACAGAACATTCTTGGAATGCTGAT |
| | | CTTTATAAGCTCATGGGACACTTCGCATGG |
| | | TGGACAGCCTTTGTTACTAATGTGAATGCG |
| | | TCATCATCTGAAGCATTTTTAATTGGATGT |
| | | AATTATCTTGGCAAACCACGCGAACAAATA |
| | | GATGGTTATGTCATGCATGCAAATTACATA |
| | | TTTTGGAGGAATACAAATCCAATTCAGTTG |
| | | TCTTCCTATTCTTTATTTGACATGAGTAAA |
| | | TTTCCCCTTAAATTAAGGGGTACTGCTGTT |
| | | ATGTCTTTAAAAGAAGGTCAAATCAATGAT |
| | | ATGATTTTATCTCTTCTTAGTAAAGGTAGA |
| | | CTTATAATTAGAGAAAACAACAGAGTTGTT |
| | | ATTTCTAGTGATGTTCTTGT |
| 302 | Helicase 16236/18039 | GGGCTGTTGGGGCTTGTGTTCTTTGCAATT |
| | | CACAGACTTCATTAAGATGTGGTGCTTGCA |
| | | TACGTAGACCATTCTTATGTTGTAAATGCT |
| | | GTTACGACCATGTCATATCAACATCACATA |
| | | AATTAGTCTTGTCTGTTAATCCGTATGTTT |
| | | GCAATGCTCCAGGTTGTGATGTCACAGATG |
| | | TGACTCAACTTTACTTAGGAGGTATGAGCT |
| | | ATTATTGTAAATCACATAAACCACCCATTA |
| | | GTTTTCCATTGTGTGCTAATGGACAAGTTT |
| | | TTGGTTTATATAAAAATACATGTGTTGGTA |
| | | GCGATAATGTTACTGACTTTAATGCAATTG |
| | | CAACATGTGACTGGACAAATGCTGGTGATT |
| | | ACATTTTAGCTAACACCTGTACTGAAAGAC |
| | | TCAAGCTTTTTGCAGCAGAAACGCTCAAAG |
| | | CTACTGAGGAGACATTTAAACTGTCTTATG |
| | | GTATTGCTACTGTACGTGAAGTGCTGTCTG |
| | | ACAGAGAATTACATCTTTCATGGGAAGTTG |
| | | GTAAACCTAGACCACCACTTAACCGAAATT |
| | | ATGTCTTTACTGGTTATCGTGTAACTAAAA |
| | | ACAGTAAAGTACAAATAGGAGAGTACACCT |
| | | TTGAAAAAGGTGACTATGGTGATGCTGTTG |
| | | TTTACCGAGGTACAACAACTTACAAATTAA |
| | | ATGTTGGTGATTATTTTGTGCTGACATCAC |
| | | ATACAGTAATGCCATTAAGTGCACCTACAC |
| | | TAGTGCCACAAGAGCACTATGTTAGAATTA |
| | | CTGGCTTATACCCAACACTCAATATCTCAG |
| | | ATGAGTTTTCTAGCAATGTTGCAAATTATC |
| | | AAAAGGTTGGTATGCAAAAGTATTCTACAC |
| | | TCCAGGGACCACCTGGTACTGGTAAGAGTC |
| | | ATTTTGCTATTGGCCTAGCTCTCTACTACC |
| | | CTTCTGCTCGCATAGTGTATACAGCTTGCT |
| | | CTCATGCCGCTGTTGATGCACTATGTGAGA |
| | | AGGCATTAAAATATTTGCCTATAGATAAAT |
| | | GTAGTAGAATTATACCTGCACGTGCTCGTG |
| | | TAGAGTGTTTTGATAAATTCAAAGTGAATT |
| | | CAACATTAGAACAGTATGTCTTTTGTACTG |
| | | TAAATGCATTGCCTGAGACGACAGCAGATA |
| | | TAGTTGTCTTTGATGAAATTTCAATGGCCA |
| | | CAAATTATGATTTGAGTGTTGTCAATGCCA |
| | | GATTACGTGCTAAGCACTATGTGTACATTG |
| | | GCGACCCTGCTCAATTACCTGCACCACGCA |
| | | CATTGCTAACTAAGGGCACACTAGAACCAG |
| | | AATATTTCAATTCAGTGTGTAGACTTATGA |
| | | AAACTATAGGTCCAGACATGTTCCTCGGAA |
| | | CTTGTCGGCGTTGTCCTGCTGAAATTGTTG |
| | | ACACTGTGAGTGCTTTGGTTTATGATAATA |
| | | AGCTTAAAGCACATAAAGACAAATCAGCTC |
| | | AATGCTTTAAAATGTTTTATAAGGGTGTTA |
| | | TCACGCATGATGTTTCATCTGCAATTAACA |
| | | GGCCACAAATAGGCGTGGTAAGAGAATTCC |
| | | TTACACGTAACCCTGCTTGGAGAAAAGCTG |
| | | TCTTTATTTCACCTTATAATTCACAGAATG |
| | | CTGTAGCCTCAAAGATTTTGGGACTACCAA |
| | | CTCAAACTGTTGATTCATCACAGGGCTCAG |
| | | AATATGACTATGTCATATTCACTCAAACCA |
| | | CTGAAACAGCTCACTCTTGTAATGTAAACA |
| | | GATTTAATGTTGCTATTACCAGAGCAAAAG |
| | | TAGGCATACTTTGCATAATGTCTGATAGAG |
| | | ACCTTTATGACAAGTTGCAATTTACAAGTC |
| | | TTGAAATTCCACGTAGGAATGTGGCAACTT |
| | | TACAA |
| 303 | Exonuclease 18040/19620 | GGGCTGAAAATGTAACAGGACTCTTTAAAG |
| | | ATTGTAGTAAGGTAATCACTGGGTTACATC |
| | | CTACACAGGCACCTACACACCTCAGTGTTG |
| | | ACACTAAATTCAAAACTGAAGGTTTATGTG |
| | | TTGACATACCTGGCATACCTAAGGACATGA |
| | | CCTATAGAAGACTCATCTCTATGATGGGTT |
| | | TTAAAATGAATTATCAAGTTAATGGTTACC |
| | | CTAACATGTTTATCACCCGCGAAGAAGCTA |
| | | TAAGACATGTACGTGCATGGATTGGCTTCG |
| | | ATGTCGAGGGGTGTCATGCTACTAGAGAAG |
| | | CTGTTGGTACCAATTTACCTTTACAGCTAG |
| | | GTTTTTCTACAGGTGTTAACCTAGTTGCTG |
| | | TACCTACAGGTTATGTTGATACACCTAATA |
| | | ATACAGATTTTTCCAGAGTTAGTGCTAAAC |
| | | CACCGCCTGGAGATCAATTTAAACACCTCA |
| | | TACCACTTATGTACAAAGGACTTCCTTGGA |
| | | ATGTAGTGCGTATAAAGATTGTACAAATGT |
| | | TAAGTGACACACTTAAAAATCTCTCTGACA |
| | | GAGTCGTATTTGTCTTATGGGCACATGGCT |
| | | TTGAGTTGACATCTATGAAGTATTTTGTGA |
| | | AAATAGGACCTGAGCGCACCTGTTGTCTAT |
| | | GTGATAGACGTGCCACATGCTTTTCCACTG |
| | | CTTCAGACACTTATGCCTGTTGGCATCATT |
| | | CTATTGGATTTGATTACGTCTATAATCCGT |
| | | TTATGATTGATGTTCAACAATGGGGTTTTA |
| | | CAGGTAACCTACAAAGCAACCATGATCTGT |
| | | ATTGTCAAGTCCATGGTAATGCACATGTAG |
| | | CTAGTTGTGATGCAATCATGACTAGGTGTC |
| | | TAGCTGTCCACGAGTGCTTTGTTAAGCGTG |
| | | TTGACTGGACTATTGAATATCCTATAATTG |
| | | GTGATGAACTGAAGATTAATGCGGCTTGTA |
| | | GAAAGGTTCAACACATGGTTGTTAAAGCTG |
| | | CATTATTAGCAGACAAATTCCCAGTTCTTC |
| | | ACGACATTGGTAACCCTAAAGCTATTAAGT |
| | | GTGTACCTCAAGCTGATGTAGAATGGAAGT |
| | | TCTATGATGCACAGCCTTGTAGTGACAAAG |
| | | CTTATAAAATAGAAGAATTATTCTATTCTT |
| | | ATGCCACACATTCTGACAAATTCACAGATG |
| | | GTGTATGCCTATTTTGGAATTGCAATGTCG |
| | | ATAGATATCCTGCTAATTCCATTGTTTGTA |
| | | GATTTGACACTAGAGTGCTATCTAACCTTA |
| | | ACTTGCCTGGTTGTGATGGTGGCAGTTTGT |
| | | ATGTAAATAAACATGCATTCCACACACCAG |
| | | CTTTTGATAAAAGTGCTTTTGTTAATTTAA |
| | | AACAATTACCATTTTTCTATTACTCTGACA |
| | | GTCCATGTGAGTCTCATGGAAAACAAGTAG |
| | | TGTCAGATATAGATTATGTACCACTAAAGT |
| | | CTGCTACGTGTATAACACGTTGCAATTTAG |
| | | GTGGTGCTGTCTGTAGACATCATGCTAATG |
| | | AGTACAGATTGTATCTCGATGCTTATAACA |
| | | TGATGATCTCAGCTGGCTTTAGCTTGTGGG |
| | | TTTACAAACAATTTGATACTTATAACCTCT |
| | | GGAACACTTTTACAAGACTTCAG |
| 304 | ORF3a 25393/26220 | GGGATGGATTTGTTTATGAGAATCTTCACA |
| | | ATTGGAACTGTAACTTTGAAGCAAGGTGAA |
| | | ATCAAGGATGCTACTCCTTCAGATTTTGTT |
| | | CGCGCTACTGCAACGATACCGATACAAGCC |
| | | TCACTCCCTTTCGGATGGCTTATTGTTGGC |
| | | GTTGCACTTCTTGCTGTTTTTCAGAGCGCT |
| | | TCCAAAATCATAACCCTCAAAAAGAGATGG |
| | | CAACTAGCACTCTCCAAGGGTGTTCACTTT |
| | | GTTTGCAACTTGCTGTTGTTGTTTGTAACA |
| | | GTTTACTCACACCTTTTGCTCGTTGCTGCT |
| | | GGCCTTGAAGCCCCTTTTCTCTATCTTTAT |
| | | GCTTTAGTCTACTTCTTGCAGAGTATAAAC |
| | | TTTGTAAGAATAATAATGAGGCTTTGGCTT |
| | | TGCTGGAAATGCCGTTCCAAAAACCCATTA |
| | | CTTTATGATGCCAACTATTTTCTTTGCTGG |
| | | CATACTAATTGTTACGACTATTGTATACCT |
| | | TACAATAGTGTAACTTCTTCAATTGTCATT |
| | | ACTTCAGGTGATGGCACAACAAGTCCTATT |
| | | TCTGAACATGACTACCAGATTGGTGGTTAT |
| | | ACTGAAAAATGGGAATCTGGAGTAAAAGAC |
| | | TGTGTTGTATTACACAGTTACTTCACTTCA |
| | | GACTATTACCAGCTGTACTCAACTCAATTG |
| | | AGTACAGACACTGGTGTTGAACATGTTACC |
| | | TTCTTCATCTACAATAAAATTGTTGATGAG |
| | | CCTGAAGAACATGTCCAAATTCACACAATC |
| | | GACGGTTCATCCGGAGTTGTTAATCCAGTA |
| | | ATGGAACCAATTTATGATGAACCGACGACG |
| | | ACTACTAGCGTGCCTTTGTAA |
| 305 | NSP1 266/805 | GGGATGGAGAGCCTTGTCCCTGGTTTCAAC |
| | | GAGAAAACACACGTCCAACTCAGTTTGCCT |
| | | GTTTTACAGGTTCGCGACGTGCTCGTACGT |
| | | GGCTTTGGAGACTCCGTGGAGGAGGTCTTA |
| | | TCAGAGGCACGTCAACATCTTAAAGATGGC |
| | | ACTTGTGGCTTAGTAGAAGTTGAAAAAGGC |
| | | GTTTTGCCTCAACTTGAACAGCCCTATGTG |
| | | TTCATCAAACGTTCGGATGCTCGAACTGCA |
| | | CCTCATGGTCATGTTATGGTTGAGCTGGTA |
| | | GCAGAACTCGAAGGCATTCAGTACGGTCGT |
| | | AGTGGTGAGACACTTGGTGTCCTTGTCCCT |
| | | CATGTGGGCGAAATACCAGTGGCTTACCGC |
| | | AAGGTTCTTCTTCGTAAGAACGGTAATAAA |
| | | GGAGCTGGTGGCCATAGTTACGGCGCCGAT |
| | | CTAAAGTCATTTGACTTAGGCGACGAGCTT |
| | | GGCACTGATCCTTATGAAGATTTTCAAGAA |
| | | AACTGGAACACTAAACATAGCAGTGGTGTT |
| | | ACCCGTGAACTCATGCGTGAGCTTAACGGA |
| | | GGG |
| 306 | NSP2 805/2719 | GGGCATACACTCGCTATGTCGATAACAACT |
| | | TCTGTGGCCCTGATGGCTACCCTCTTGAGT |
| | | GCATTAAAGACCTTCTAGCACGTGCTGGTA |
| | | AAGCTTCATGCACTTTGTCCGAACAACTGG |
| | | ACTTTATTGACACTAAGAGGGGTGTATACT |
| | | GCTGCCGTGAACATGAGCATGAAATTGCTT |
| | | GGTACACGGAACGTTCTGAAAAGAGCTATG |
| | | AATTGCAGACACCTTTTGAAATTAAATTGG |
| | | CAAAGAAATTTGACACCTTCAATGGGGAAT |
| | | GTCCAAATTTTGTATTTCCCTTAAATTCCA |
| | | TAATCAAGACTATTCAACCAAGGGTTGAAA |
| | | AGAAAAAGCTTGATGGCTTTATGGGTAGAA |
| | | TTCGATCTGTCTATCCAGTTGCGTCACCAA |
| | | ATGAATGCAACCAAATGTGCCTTTCAACTC |
| | | TCATGAAGTGTGATCATTGTGGTGAAACTT |
| | | CATGGCAGACGGGCGATTTTGTTAAAGCCA |
| | | CTTGCGAATTTTGTGGCACTGAGAATTTGA |
| | | CTAAAGAAGGTGCCACTACTTGTGGTTACT |
| | | TACCCCAAAATGCTGTTGTTAAAATTTATT |
| | | GTCCAGCATGTCACAATTCAGAAGTAGGAC |
| | | CTGAGCATAGTCTTGCCGAATACCATAATG |
| | | AATCTGGCTTGAAAACCATTCTTCGTAAGG |
| | | GTGGTCGCACTATTGCCTTTGGAGGCTGTG |
| | | TGTTCTCTTATGTTGGTTGCCATAACAAGT |
| | | GTGCCTATTGGGTTCCACGTGCTAGCGCTA |
| | | ACATAGGTTGTAACCATACAGGTGTTGTTG |
| | | GAGAAGGTTCCGAAGGTCTTAATGACAACC |
| | | TTCTTGAAATACTCCAAAAAGAGAAAGTCA |
| | | ACATCAATATTGTTGGTGACTTTAAACTTA |
| | | ATGAAGAGATCGCCATTATTTTGGCATCTT |
| | | TTTCTGCTTCCACAAGTGCTTTTGTGGAAA |
| | | CTGTGAAAGGTTTGGATTATAAAGCATTCA |
| | | AACAAATTGTTGAATCCTGTGGTAATTTTA |
| | | AAGTTACAAAAGGAAAAGCTAAAAAAGGTG |
| | | CCTGGAATATTGGTGAACAGAAATCAATAC |
| | | TGAGTCCTCTTTATGCATTTGCATCAGAGG |
| | | CTGCTCGTGTTGTACGATCAATTTTCTCCC |
| | | GCACTCTTGAAACTGCTCAAAATTCTGTGC |
| | | GTGTTTTACAGAAGGCCGCTATAACAATAC |
| | | TAGATGGAATTTCACAGTATTCACTGAGAC |
| | | TCATTGATGCTATGATGTTCACATCTGATT |
| | | TGGCTACTAACAATCTAGTTGTAATGGCCT |
| | | ACATTACAGGTGGTGTTGTTCAGTTGACTT |
| | | CGCAGTGGCTAACTAACATCTTTGGCACTG |
| | | TTTATGAAAAACTCAAACCCGTCCTTGATT |
| | | GGCTTGAAGAGAAGTTTAAGGAAGGTGTAG |
| | | AGTTTCTTAGAGACGGTTGGGAAATTGTTA |
| | | AATTTATCTCAACCTGTGCTTGTGAAATTG |
| | | TCGGTGGACAAATTGTCACCTGTGCAAAGG |
| | | AAATTAAGGAGAGTGTTCAGACATTCTTTA |
| | | AGCTTGTAAATAAATTTTTGGCTTTGTGTG |
| | | CTGACTCTATCATTATTGGTGGAGCTAAAC |
| | | TTAAAGCCTTGAATTTAGGTGAAACATTTG |
| | | TCACGCACTCAAAGGGATTGTACAGAAAGT |
| | | GTGTTAAATCCAGAGAAGAAACTGGCCTAC |
| | | TCATGCCTCTAAAAGCCCCAAAAGAAATTA |
| | | TCTTCTTAGAGGGAGAAACACTTCCCACAG |
| | | AAGTGTTAACAGAGGAAGTTGTCTTGAAAA |
| | | CTGGTGATTTACAACCATTAGAACAACCTA |
| | | CTAGTGAAGCTGTTGAAGCTCCATTGGTTG |
| | | GTACACCAGTTTGTATTAACGGGCTTATGT |
| | | TGCTCGAAATCAAAGACACAGAAAAGTACT |
| | | GTGCCCTTGCACCTAATATGATGGTAACAA |
| | | ACAATACCTTCACACTCAAAGGCGGT |
| 307 | NSP3 3027/4791 | GGGCTGGTGAGTTTAAATTGGCTTCACATA |
| | | TGTATTGTTCTTTCTACCCTCCAGATGAGG |
| | | ATGAAGAAGAAGGTGATTGTGAAGAAGAAG |
| | | AGTTTGAGCCATCAACTCAATATGAGTATG |
| | | GTACTGAAGATGATTACCAAGGTAAACCTT |
| | | TGGAATTTGGTGCCACTTCTGCTGCTCTTC |
| | | AACCTGAAGAAGAGCAAGAAGAAGATTGGT |
| | | TAGATGATGATAGTCAACAAACTGTTGGTC |
| | | AACAAGACGGCAGTGAGGACAATCAGACAA |
| | | CTACTATTCAAACAATTGTTGAGGTTCAAC |
| | | CTCAATTAGAGATGGAACTTACACCAGTTG |
| | | TTCAGACTATTGAAGTGAATAGTTTTAGTG |
| | | GTTATTTAAAACTTACTGACAATGTATACA |
| | | TTAAAAATGCAGACATTGTGGAAGAAGCTA |
| | | AAAAGGTAAAACCAACAGTGGTTGTTAATG |
| | | CAGCCAATGTTTACCTTAAACATGGAGGAG |
| | | GTGTTGCAGGAGCCTTAAATAAGGCTACTA |
| | | ACAATGCCATGCAAGTTGAATCTGATGATT |
| | | ACATAGCTACTAATGGACCACTTAAAGTGG |
| | | GTGGTAGTTGTGTTTTAAGCGGACACAATC |
| | | TTGCTAAACACTGTCTTCATGTTGTCGGCC |
| | | CAAATGTTAACAAAGGTGAAGACATTCAAC |
| | | TTCTTAAGAGTGCTTATGAAAATTTTAATC |
| | | AGCACGAAGTTCTACTTGCACCATTATTAT |
| | | CAGCTGGTATTTTTGGTGCTGACCCTATAC |
| | | ATTCTTTAAGAGTTTGTGTAGATACTGTTC |
| | | GCACAAATGTCTACTTAGCTGTCTTTGATA |
| | | AAAATCTCTATGACAAACTTGTTTCAAGCT |
| | | TTTTGGAAATGAAGAGTGAAAAGCAAGTTG |
| | | AACAAAAGATCGCTGAGATTCCTAAAGAGG |
| | | AAGTTAAGCCATTTATAACTGAAAGTAAAC |
| | | CTTCAGTTGAACAGAGAAAACAAGATGATA |
| | | AGAAAATCAAAGCTTGTGTTGAAGAAGTTA |
| | | CAACAACTCTGGAAGAAACTAAGTTCCTCA |
| | | CAGAAAACTTGTTACTTTATATTGACATTA |
| | | ATGGCAATCTTCATCCAGATTCTGCCACTC |
| | | TTGTTAGTGACATTGACATCACTTTCTTAA |
| | | AGAAAGATGCTCCATATATAGTGGGTGATG |
| | | TTGTTCAAGAGGGTGTTTTAACTGCTGTGG |
| | | TTATACCTACTAAAAAGGCTGGTGGCACTA |
| | | CTGAAATGCTAGCGAAAGCTTTGAGAAAAG |
| | | TGCCAACAGACAATTATATAACCACTTACC |
| | | CGGGTCAGGGTTTAAATGGTTACACTGTAG |
| | | AGGAGGCAAAGACAGTGCTTAAAAAGTGTA |
| | | AAAGTGCCTTTTACATTCTACCATCTATTA |
| | | TCTCTAATGAGAAGCAAGAAATTCTTGGAA |
| | | CTGTTTCTTGGAATTTGCGAGAAATGCTTG |
| | | CACATGCAGAAGAAACACGCAAATTAATGC |
| | | CTGTCTGTGTGGAAACTAAAGCCATAGTTT |
| | | CAACTATACAGCGTAAATATAAGGGTATTA |
| | | AAATACAAGAGGGTGTGGTTGATTATGGTG |
| | | CTAGATTTTACTTTTACACCAGTAAAACAA |
| | | CTGTAGCGTCACTTATCAACACACTTAACG |
| | | ATCTAAATGAAACTCTTGTTACAATGCCAC |
| | | TTGGCTATGTAACACATGGCTTAAATTTGG |
| | | AAGAAGCTGCTCGGTATATGAGATCTCTCA |
| | | AAGTGCCAGCTACAGTTTCTGTTTCTTCAC |
| | | CTGATGCTGTTACAGCGTATAATGGTTATC |
| | | TTACTTCTTCTTCTAAAACACCTGAAGAAC |
| | | ATTTTATTGAAACCATCTCACTTGCTGG |
| 308 | RCA18b | TCCCCATTTATTATAGGCATTAACAATGAA |
| | | TGTTAGAGTTTTTCATTAGGA |
| 309 | RCA196 | TCCCCATTTATTAATTTTTGATGAAACTGT |
| | | CGTTAGAGTTTTTCATTAGGA |
| 310 | RCA20b | TCCCCATTTATCTACAGTAGCTCCTCTAGT |
| | | GGTTAGAGTTTTTCATTAGGA |
| 311 | RCA21b | TCCCCATTTATTAAGGTGAGGGTTTTCTAC |
| | | AGTTAGAGTTTTTCATTAGGA |
| 312 | RCA22b | TCCCCATTTATCCATTTCACTCAATACTTG |
| | | AGTTAGAGTTTTTCATTAGGA |
| 313 | RCA23b | TCCCCATTTATCCACATGAACCATTAAGGA |
| | | AGTTAGAGTTTTTCATTAGGA |
| 314 | RCA24b | TCCCCATTTATTGAGGTGCAGTTCGAGCAT |
| | | CGTTAGAGTTTTTCATTAGGA |
| 315 | RCA25b | TCCCCATTTATTAAACACCAAGAGTCAGTC |
| | | TGTTAGAGTTTTTCATTAGGA |
| 316 | RCA26b | TCCCCATTTATCTTTTTAAGAACAACTTCA |
| | | GGTTAGAGTTTTTCATTAGGA |
| 317 | RCA27b | TCCCCATTTATTAGCTTGATCAGCCATCTT |
| | | TGTTAGAGTTTTTCATTAGGA |
| 318 | RCA28b | TCCCCATTTATTAGCACTAGTAACTTTTGC |
| | | CGTTAGAGTTTTTCATTAGGA |
| 319 | RCA29b | TCCCCATTTATTAGTCTGGTATGACAACCA |
| | | TGTTAGAGTTTTTCATTAGGA |
| 320 | RCA30b | TCCCCATTTATTTGTCCATACTAATTTCAC |
| | | TGTTAGAGTTTTTCATTAGGA |
| 321 | RCA31b | TCCCCATTTATCGGCTTCTCCAATTAATGT |
| | | GGTTAGAGTTTTTCATTAGGA |
| 322 | RCA32b | TCCCCATTTATTTCAAAAGGTGATTCCTTA |
| | | AGTTAGAGTTTTTCATTAGGA |
| 323 | RCA33b | TCCCCATTTATTTTGTCCAGTCACATGTTG |
| | | CGTTAGAGTTTTTCATTAGGA |
| 324 | RCA34b | TCCCCATTTATTGTAATTCTCTGTCAGACA |
| | | GGTTAGAGTTTTTCATTAGGA |
| 325 | RCA35b | TCCCCATTTATCAAAATAATCACCAACATT |
| | | TGTTAGAGTTTTTCATTAGGA |
| 326 | RCA36b | TCCCCATTTATTAAGTCTACACACTGAATT |
| | | GGTTAGAGTTTTTCATTAGGA |
| 327 | RCA37b | TCCCCATTTATTCTCCAGGCGGTGGTTTAG |
| | | CGTTAGAGTTTTTCATTAGGA |
| 328 | RCA38b | TCCCCATTTATCGACTCTGTCAGAGAGATT |
| | | TGTTAGAGTTTTTCATTAGGA |
| 329 | RCA39b | TCCCCATTTATCCTTTCTACAAGCCGCATT |
| | | AGTTAGAGTTTTTCATTAGGA |
| 330 | RCA40b | TCCCCATTTATTGTCGTGAAGAACTGGGAA |
| | | TGTTAGAGTTTTTCATTAGGA |
| 331 | RCA41b | TCCCCATTTATCTCACATGGACTGTCAGAG |
| | | TGTTAGAGTTTTTCATTAGGA |
| 332 | RCA42b | TCCCCATTTATTTCTCAGTGCCACAAAATT |
| | | CGTTAGAGTTTTTCATTAGGA |
| 333 | RCA43b | TCCCCATTTATCAGAATTTTGAGCAGTTTC |
| | | AGTTAGAGTTTTTCATTAGGA |
| 334 | RCA44b | TCCCCATTTATCTTCTCTTCAAGCCAATCA |
| | | AGTTAGAGTTTTTCATTAGGA |
| 335 | RCA45b | TCCCCATTTATCACACTTTCTGTACAATCC |
| | | CGTTAGAGTTTTTCATTAGGA |
| 336 | RCA46b | TCCCCATTTATCAATCACCTTCTTCTTCAT |
| | | CGTTAGAGTTTTTCATTAGGA |
| 337 | RCA47b | TCCCCATTTATTGGTGCAAGTAGAACTTCG |
| | | TGTTAGAGTTTTTCATTAGGA |
| 338 | RCA48b | TCCCCATTTATCAAGAGTGGCAGAATCTGG |
| | | AGTTAGAGTTTTTCATTAGGA |
| 339 | RCA49b | TCCCCATTTATTGGAGCATCTTTCTTTAAG |
| | | AGTTAGAGTTTTTCATTAGGA |
| 340 | RCA50b | TCCCCATTTATCACCCTCTTGAACAACATC |
| | | AGTTAGAGTTTTTCATTAGGA |
| 341 | RCA51b | TCCCCATTTATTTTTCTTTTGTAACATTTT |
| | | TGTTAGAGTTTTTCATTAGGA |
| 342 | RCA52b | TCCCCATTTATTTTGCATGCATGACATAAC |
| | | CGTTAGAGTTTTTCATTAGGA |

For the sequences in Table 1, all suffix variants (e.g. N_CDCn1_GU1_1023b to N_CDCn1_GU1_1023g) target the same dinucleotide junction on the RNA, but vary in modifications to the DNAzyme binding arms or catalytic core. “b” suffixes have corrected catalytic cores, where the original sequences had an error. “c” suffixes have 11+7 binding arms referring to the number of pairing bases 5′ and 3′ of the cleavage sites. “d” suffixes have 12+8 binding arms. “e” suffixes have 13+8 binding arms. “f” suffixes have 15+8 binding arms. “g” suffixes have 20+8 binding arms. The sequences in Table 1 with “_DNA” suffix are control DNA primers corresponding to the priming cleavage product that would be generated by a given DNAzyme candidate. These are positive control primers to test RCA templates. “dZ” prefixes are 10-23 core, and “dY” prefixes are 8-17 core. The “a” suffixes for the dZ sequence DNAzymes are 15+8 binding arms and were used for the cleavage fragment screening described herein. In particular, at least these specific variants were screened: n1GU1=#15; n1GU3=#19; n2AU6=#22; n2AU7=#25; n3AU10=#28; n3GU5=#31; S_Japan_GU1=#40; and S_Japan_AU11=#43.

TABLE 2
SARS-COV-2 RNA genome DNAzyme cleavage positions.

| Sequence | | Cleavage Site Position |
|---|---|---|
| ID | | Referenced to GenBank |
| Number | Name | MN908947.3 |
| 10 | N_CDCn1_GU1_1023b | 28321G-28322U |
| 11 | N_CDCn1_GU1_1023c (GU1c) | 28321G-28322U |
| 12 | N_CDCn1_GU1_1023d | 28321G-28322U |
| 13 | N_CDCn1_GU1_1023e | 28321G-28322U |
| 14 | N_CDCn1_GU1_1023f | 28321G-28322U |
| 15 | N_CDCn1_GU1_1023g | 28321G-28322U |
| 17 | N_CDCn1_GU3_1023b | 28350G-28351U |
| 18 | N_CDCn1_GU3_1023c | 28350G-28351U |
| 19 | N_CDCn1_GU3_1023f | 28350G-28351U |
| 21 | N_CDCn2_AU6_1023b | 28704A-28705U |
| 22 | N_CDCn2_AU6_1023f | 28704A-28705U |
| 24 | N_CDCn2_AU7_1023b | 28722A-28723U |
| 25 | N_CDCn2_AU7_1023f | 28722A-28723U |
| 27 | N_CDCn3_AU10_1023b | 29172A-29173U |
| 28 | N_CDCn3_AU10_1023f | 29172A-29173U |
| 30 | N_CDCn3_GU5_023b | 29212G-29213U |
| 31 | N_CDCn3_GU5_1023f | 29212G-29213U |
| 33 | ORF1ab_CCDC_GU4_1023b | 13493G-13494U |
| 34 | ORF1ab_CCDC_GU4_1023f | 13493G-13494U |
| 36 | ORF1ab_CCDC_AU3_1023b | 13549A-13550U |
| 37 | ORF1ab_CCDC_AU3_1023f | 13549A-13550U |
| 39 | S_Japan_GU1_1023b | 24390G-24391U |
| 40 | S_Japan_GU1_1023f | 24390G-24391U |
| 42 | S_Japan_AU11_1023b | 24551A-24552U |
| 43 | S_Japan_AU11_1023f | 24551A-24552U |
| 45 | E_Germany_AU3_1023b | 26319A-26320U |
| 46 | E_Germany_AU3_1023f | 26319A-26320U |
| 48 | E_Germany_AU5_1023b | 26358A-26359U |
| 49 | E_Germany_AU5_1023f | 26358A-26359U |
| 51 | N_CDCn2-3_M1_1023b | 28704A-28705U |
| | | 29172A-29173U |
| 63 | dZ_28692a | 28692A-28693U |
| 64 | dZ_28734a | 28734A-28735U |
| 65 | dZ_28771a | 28771A-28772U |
| 66 | dZ_28851a | 28851G-28852U |
| 67 | dZ_21744a | 21744A-21745U |
| 68 | dZ_21768a | 21768A-21769U |
| 69 | dZ_21969a | 21969G-21970U |
| 70 | dZ_22161a | 22161A-22162U |
| 71 | dZ_22614a | 22164A-22165U |
| 72 | dZ_23847a | 23849A-24850U |
| 73 | dZ_24178a | 24178A-24179U |
| 74 | dZ_24468a | 24468A-24469U |
| 75 | dZ_24710a | 24710A-24711U |
| 76 | dZ_25097a | 25097A-25098U |
| 77 | dZ_25271a | 25271A-25272U |
| 78 | dZ_13533a | 13533A-13534U |
| 79 | dZ_13625a | 13625A-13626U |
| 80 | dZ_13726a | 13726G-13727U |
| 81 | dZ_14172a | 14172A-17173U |
| 82 | dZ_14578a | 14578A-14579U |
| 83 | dZ_14829a | 14829G-14830U |
| 84 | dZ_14984a | 14984A-14985U |
| 85 | dZ_15029a | 15029A-15030U |
| 86 | dZ_15165a | 15165G-15166U |
| 87 | dZ_15202a | 15202G-15203U |
| 88 | dZ_15282a | 15282A-15283U |
| 89 | dZ_15506a | 15506A-155070 |
| 90 | dZ_15439a | 15439G-15440U |
| 91 | dZ_15703a | 15703A-15704U |
| 92 | dZ_15921a | 15921G-15922U |
| 93 | dZ_26666a | 26666A-26667U |
| 94 | dZ_26718a | 26718G-26719U |
| 95 | dZ_26874a | 26874A-26875U |
| 96 | dZ_27137a | 27137A-27137U |
| 105 | dZ_10098a | 10098G-10099U |
| 106 | dZ_10140a | 10140G-10141U |
| 107 | dZ_10176a | 10176A-10177U |
| 108 | dZ_10256a | 10256G-10257U |
| 109 | dZ_10325a | 10325G-10326U |
| 110 | dZ_10338a | 10338A-10339U |
| 111 | dZ_10442a | 10442A-10443U |
| 112 | dZ_10491a | 10491G-10492U |
| 113 | dZ_10599a | 10599A-10600U |
| 114 | dZ_10800a | 10800A-10801U |
| 115 | dZ_11062a | 11062A-11063U |
| 116 | dZ_11085a | 11085A-11086U |
| 117 | dZ_11111a | 11111A-11112U |
| 118 | dZ_11217a | 11217A-11218U |
| 119 | dZ_11270a | 11270A-11271U |
| 120 | dZ_11342a | 11342A-11343U |
| 121 | dZ_11502a | 11502G-11503U |
| 122 | dZ_11521a | 11521G-11522U |
| 123 | dZ_11567a | 11567A-11568U |
| 124 | dZ_11616a | 11616G-11617U |
| 125 | dZ_11697a | 11697A-11698U |
| 126 | dZ_11730a | 11730A-11731U |
| 127 | dZ_12156a | 12156A-12157U |
| 128 | dZ_12174a | 12174A-12175U |
| 129 | dZ_12202a | 12202G-12203U |
| 130 | dZ_12262a | 12262G-12263U |
| 131 | dZ_12290a | 12290A-12291U |
| 132 | dZ_12299a | 12299A-12300U |
| 133 | dZ_12350a | 12350A-12351U |
| 134 | dZ_12359a | 12359A-12360U |
| 135 | dZ_12495a | 12495A-12496U |
| 136 | dZ_12557a | 12557A-12558U |
| 137 | dZ_12618a | 12618A-12619U |
| 138 | dZ_19699a | 19699A-19700U |
| 139 | dZ_19743a | 19743A-19744U |
| 140 | dZ_19825a | 19825G-19826U |
| 141 | dZ_19892a | 19892A-19893U |
| 142 | dZ_19915a | 19915A-19916U |
| 143 | dZ_19963a | 19963A-19964U |
| 144 | dZ_20103a | 20103G-20104U |
| 145 | dZ_20134a | 20134G-20135U |
| 146 | dZ_20156a | 20156A-20157U |
| 147 | dZ_20184a | 20184A-20185U |
| 148 | dZ_20216a | 20216A-20217U |
| 149 | dZ_20251a | 20251A-20252U |
| 150 | dZ_20276a | 20276A-20277U |
| 151 | dZ_20412a | 20412A-20413U |
| 152 | dZ_20426a | 20426A-20427U |
| 153 | dZ_20511a | 20511A-20512U |
| 154 | dZ_16334a | 16334A-16335U |
| 155 | dZ_16485a | 16485G-16486U |
| 156 | dZ_16501a | 16501G-16502U |
| 157 | dZ_16583a | 16583A-16584U |
| 158 | dZ_16727a | 16727A-16728U |
| 159 | dZ_16890a | 16890A-16891U |
| 160 | dZ_16912a | 16912G-16913U |
| 161 | dZ_16925a | 16925A-16926U |
| 162 | dZ_16981a | 16981A-16982U |
| 163 | dZ_17207a | 17207A-17208U |
| 164 | dZ_17344a | 17344A-17345U |
| 165 | dZ_17378a | 17378A-17379U |
| 166 | dZ_17406a | 17406A-17407U |
| 167 | dZ_17498a | 17498A-17499U |
| 168 | dZ_17522a | 17522A-17523U |
| 169 | dZ_17567a | 17567G-17568U |
| 170 | dZ_17658a | 17658G-17659U |
| 171 | dZ_17713a | 17713A-17714U |
| 172 | dZ_17730a | 17730A-17731U |
| 173 | dZ_17780a | 17780A-17781U |
| 174 | dZ_18135a | 18135A-18136U |
| 175 | dZ_18153a | 18153A-18154U |
| 176 | dZ_18235a | 18235G-18236U |
| 177 | dZ_18259a | 18259A-18260U |
| 178 | dZ_18391a | 18391G-18392U |
| 179 | dZ_18470a | 18470A-18471U |
| 180 | dZ_18498a | 18498G-18499U |
| 181 | dZ_18535a | 18535A-18536U |
| 182 | dZ_18583a | 18583G-18584U |
| 183 | dZ_18640a | 18640A-18641U |
| 184 | dZ_18791a | 18791G-18792U |
| 185 | dZ_18818a | 18818A-18819U |
| 186 | dZ_18919a | 18919A-18920U |
| 187 | dZ_18941a | 18941A-18942U |
| 188 | dZ_18973a | 18973G-18974U |
| 189 | dZ_19033a | 19033G-19034U |
| 190 | dZ_19182a | 19182A-19183U |
| 191 | dZ_19334a | 19334A-19335U |
| 192 | dZ_19376a | 19376A-19377U |
| 193 | dZ_19398a | 19398G-19399U |
| 194 | dZ_15501a | 15501A-15502U |
| 195 | dZ_25524a | 25524A-25525U |
| 196 | dZ_25540a | 25540G-25541U |
| 197 | dZ_25556a | 25556G-25557U |
| 198 | dZ_25596a | 25596A-25597U |
| 199 | dZ_25621a | 25621G-25622U |
| 200 | dZ_25647a | 25647G-25648U |
| 201 | dZ_25660a | 25660G-25661U |
| 202 | dZ_25765a | 25765A-25766U |
| 203 | dZ_25806a | 25806A-25807U |
| 204 | dZ_25826a | 25826A-25827U |
| 205 | dZ_25847a | 25847A-25848U |
| 206 | dZ_25937a | 25937A-25938U |
| 207 | dZ_25967a | 25967A-25968U |
| 208 | dZ_26072a | 26072A-26073U |
| 209 | dZ_26155a | 26155G-26156U |
| 210 | dZ_341a | 341G-342U |
| 211 | dZ_355a | 355G-356U |
| 212 | dZ_426a | 426G-427U |
| 213 | dZ_468a | 468A-469U |
| 214 | dZ_483a | 483G-484U |
| 215 | dZ_507a | 507A-508U |
| 216 | dZ_558a | 558G-559U |
| 217 | dZ_578a | 578G-579U |
| 218 | dZ_648a | 648A-649U |
| 219 | dZ_688a | 688G-689U |
| 220 | dZ_765a | 765G-766U |
| 221 | dZ_20716a | 20716A-20717U |
| 222 | dZ_20730a | 20730G-20731U |
| 223 | dZ_20756a | 20756G-20757U |
| 224 | dZ_20788a | 20788G-20789U |
| 225 | dZ_20817a | 20817A-20818U |
| 226 | dZ_20851a | 20851A-20852U |
| 227 | dZ_20882a | 20882A-20883U |
| 228 | dZ_20954a | 20954A-20955U |
| 229 | dZ_20992a | 20992A-20993U |
| 230 | dZ_21086a | 21086A-21087U |
| 231 | dZ_21127a | 21127A-21128U |
| 232 | dZ_21115a | 21115A-21116U |
| 233 | dZ_21238a | 21238G-21239U |
| 234 | dZ_21290a | 21290A-21291U |
| 235 | dZ_21313a | 21313A-21314U |
| 236 | dZ_21338a | 21338A-21339U |
| 237 | dZ_21345a | 21345A-21346U |
| 238 | dZ_21390a | 21390A-21391U |
| 239 | dZ_21467a | 21467A-21468U |
| 240 | dZ_846a | 846A-847U |
| 241 | dZ_866a | 866A-867U |
| 242 | dZ_910a | 910G-911U |
| 243 | dZ_1015a | 1015A-1016U |
| 244 | dZ_1051a | 1051A-1052U |
| 245 | dZ_1080a | 1080A-1081U |
| 246 | dZ_1168a | 1168A-1169U |
| 247 | dZ_1210a | 1210G-1211U |
| 248 | dZ_1243a | 1243G-1244U |
| 249 | dZ_1308a | 1308A-1309U |
| 250 | dZ_1338a | 1338G-1339U |
| 251 | dZ_1367a | 1367A-1368U |
| 252 | dZ_1431a | 1431A-1432U |
| 253 | dZ_1475a | 1475A-1476U |
| 254 | dZ_1599a | 1599G-1600U |
| 255 | dZ_1719a | 1719G-1720U |
| 256 | dZ_1759a | 1759A-1760U |
| 257 | dZ_1796a | 1796G-1797U |
| 258 | dZ_1846a | 1846A-1847U |
| 259 | dZ_1940a | 1940G-1941U |
| 260 | dZ_2020a | 2020G-2021U |
| 261 | dZ_2127a | 2127A-2128U |
| 262 | dZ_2167a | 2167G-2168U |
| 263 | dZ_2244a | 2244G-2245U |
| 264 | dZ_2276a | 2276A-2277U |
| 265 | dZ_2376a | 2376A-2377U |
| 266 | dZ_2426a | 2426G-2427U |
| 267 | dZ_3030a | 3030G-3031U |
| 268 | dZ_3072a | 3072G-3073U |
| 269 | dZ_3124a | 3124A-3125U |
| 270 | dZ_3207a | 3207A-3208U |
| 271 | dZ_3377a | 3377G-3378U |
| 272 | dZ_3419a | 3419G-3420U |
| 273 | dZ_3512a | 3512A-3513U |
| 274 | dZ_3531a | 3531A-3532U |
| 275 | dZ_3647a | 3647A-3648U |
| 276 | dZ_3681a | 3681A-3682U |
| 277 | dZ_3706a | 3706A-3707U |
| 278 | dZ_3755a | 3755G-3756U |
| 279 | dZ_3782a | 3782G-3783U |
| 280 | dZ_3813a | 3813A-3814U |
| 281 | dZ_3908a | 3908A-3909U |
| 282 | dZ_3960a | 3960A-3961U |
| 283 | dZ_4044a | 4044A-4045U |
| 284 | dZ_4076a | 4076G-4077U |
| 285 | dZ_4118a | 4118A-4119U |
| 286 | dZ_4148a | 4148G-4149U |
| 287 | dZ_4239a | 4239A-4240U |
| 288 | dZ_4269a | 4269A-4270U |
| 289 | dZ_4298a | 4298G-4299U |
| 290 | dZ_4317a | 4317G-4318U |
| 291 | dZ_4343a | 4343A-4344U |
| 292 | dZ_4386a | 4386A-4387U |
| 293 | dZ_4528a | 4528A-4529U |
| 294 | dZ_4590a | 4590A-4591U |
| 295 | dZ_4731a | 4731A-4732U |

TABLE 3
RNA substrates and complementary DNAzymes.

| Sequence | | |
|---|---|---|
| ID | | |
| Number | Name | Complementary DNAzymes |
| 1 | n1 RNA | N_CDCn1_GU1_1023b |
| | | N_CDCn1_GU1_1023c (GU1c) |
| | | N_CDCn1_GU1_1023d |
| | | N_CDCn1_GU1_1023e |
| | | N_CDCn1_GU1_1023f |
| | | N_CDCn1_GU1_1023g |
| | | N_CDCn1_GU3_1023b |
| | | N_CDCn1_GU3_1023c |
| | | N_CDCn1_GU3_1023f |
| 2 | n2 RNA | N_CDCn2_AU6_1023b |
| | | N_CDCn2_AU6_1023f |
| | | N_CDCn2_AU7_1023b |
| | | N_CDCn2_AU7_1023f |
| | | N_CDCn2-3_M1_1023b |
| 3 | n3 RNA | N_CDCn3_AU10_1023b |
| | | N_CDCn3_AU10_1023f |
| | | N_CDCn3_GU5_1023b |
| | | N_CDCn3_GU5_1023f |
| | | N_CDCn2-3_M1_1023b |
| 4 | nCov_ORF1ab_ | ORF1ab_CCDC_GU4_1023b |
| | 13470_T7_RNA | ORF1ab_CCDC_GU4_1023f |
| 5 | nCov_ORF1ab_ | ORF1ab_CCDC_AU3_1023b |
| | 13513_T7_RNA | ORF1ab_CCDC_AU3_1023f |
| 6 | nCov_S_24356_ | S_Japan_GU1_1023b |
| | T7_RNA | S_Japan_GU1_1023f |
| 7 | nCov_S_24526_ | S_Japan_AU11_1023b |
| | T7_RNA | S_Japan_AU11_1023f |
| 8 | nCov_E_26286_ | E_Germany_AU3_1023b |
| | T7_RNA | E_Germany_AU3_1023f |
| 9 | nCov_E_26329_ | E_Germany_AU5_1023b |
| | T7_RNA | E_Germany_AU5_1023f |
| 97 | Nucleocapsid Full | N_CDCn1_GU1_1023b |
| | | N_CDCn1_GU1_1023c (GU1c) |
| | | N_CDCn1_GU1_1023d |
| | | N_CDCn1_GU1_1023e |
| | | N_CDCn1_GU1_1023f |
| | | N_CDCn1_GU1_1023g |
| | | N_CDCn1_GU3_1023b |
| | | N_CDCn1_GU3_1023c |
| | | N_CDCn1_GU3_1023f |
| | | N_CDCn2_AU6_1023b |
| | | N_CDCn2_AU6_1023f |
| | | N_CDCn2_AU7_1023b |
| | | N_CDCn2_AU7_1023f |
| | | N_CDCn2-3_M1_1023b |
| | | N_CDCn3_AU10_1023b |
| | | N_CDCn3_AU10_1023f |
| | | N_CDCn3_GU5_1023b |
| | | N_CDCn3_GU5_1023f |
| | | N_CDCn2-3_M1_1023b |
| | | dZ_28692 |
| | | dZ_28734 |
| | | dZ_28771 |
| | | dZ_28851 |
| 98 | RdRp 13469/14676 | ORF1ab_CCDC_GU4_1023 |
| | | dZ_13533 |
| | | ORF1ab_CCDC_AU3_1023 |
| | | dZ_13625 |
| | | dZ_13726 |
| | | dZ_14172 |
| | | dZ_14578 |
| 99 | RdRp 14793/16197 | dZ_14829 |
| | | dZ_14984 |
| | | dZ_15029 |
| | | dZ_15165 |
| | | dZ_15202 |
| | | dZ_15283 |
| | | dZ_15439 |
| | | dZ_15506 |
| | | dZ_15703 |
| | | dZ_15921 |
| 100 | Spike 21655/22420 | dZ_21744 |
| | | dZ_21768 |
| | | dZ_21969 |
| | | dZ_22161 |
| 101 | Spike 22420/23122 | dZ_22614 |
| 102 | Spike 23436/23911 | dZ_23847 |
| 103 | Spike 24108/24665 | dZ_24178 |
| | | S_Japan_GU1_1023 |
| | | dZ_22468 |
| | | S_Japan_AU11_1023 |
| 104 | Spike 24669/25343 | dZ_24710 |
| | | dZ_25097 |
| | | dZ_25271 |
| 296 | Membrane 26523/27192 | dZ_26666a |
| | | dZ_26718a |
| | | dZ_26874a |
| | | dZ_27137a |
| 297 | 3CL 10054/10972 | dZ_10098a |
| | | dZ_10140a |
| | | dZ_10176a |
| | | dZ_10256a |
| | | dZ_10325a |
| | | dZ_10338a |
| | | dZ_10442a |
| | | dZ_10491a |
| | | dZ_10599a |
| | | dZ_10800a |
| 298 | NSP6 10992/11832 | dZ_11062a |
| | | dZ_11085a |
| | | dZ_11111a |
| | | dZ_11217a |
| | | dZ_11270a |
| | | dZ_11342a |
| | | dZ_11502a |
| | | dZ_11521a |
| | | dZ_11567a |
| | | dZ_11616a |
| | | dZ_11697a |
| | | dZ_11730a |
| 299 | NSP8 12098/12679 | dZ_12156a |
| | | dZ_12174a |
| | | dZ_12202a |
| | | dZ_12262a |
| | | dZ_12290a |
| | | dZ_12299a |
| | | dZ_12350a |
| | | dZ_12359a |
| | | dZ_12495a |
| | | dZ_12557a |
| | | dZ_12618a |
| 300 | NSP15 19620/20659 | dZ_19699a |
| | | dZ_19743a |
| | | dZ_19825a |
| | | dZ_19892a |
| | | dZ_19915a |
| | | dZ_19963a |
| | | dZ_20103a |
| | | dZ_20134a |
| | | dZ_20156a |
| | | dZ_20184a |
| | | dZ_20216a |
| | | dZ_20251a |
| | | dZ_20276a |
| | | dZ_20412a |
| | | dZ_20426a |
| | | dZ_20511a |
| 301 | Methyl-Transferase | dZ_20716a |
| | 20659/21545 | dZ_20730a |
| | | dZ_20756a |
| | | dZ_20788a |
| | | dZ_20817a |
| | | dZ_20851a |
| | | dZ_20882a |
| | | dZ_20954a |
| | | dZ_20992a |
| | | dZ_21086a |
| | | dZ_21127a |
| | | dZ_21115a |
| | | dZ_21238a |
| | | dZ_21290a |
| | | dZ_21313a |
| | | dZ_21338a |
| | | dZ_21345a |
| | | dZ_21390a |
| | | dZ_21467a |
| 302 | Helicase 16236/18039 | dZ_16334a |
| | | dZ_16485a |
| | | dZ_16501a |
| | | dZ_16583a |
| | | dZ_16727a |
| | | dZ_16890a |
| | | dZ_16912a |
| | | dZ_16925a |
| | | dZ_16981a |
| | | dZ_17207a |
| | | dZ_17344a |
| | | dZ_17378a |
| | | dZ_17406a |
| | | dZ_17498a |
| | | dZ_17522a |
| | | dZ_17567a |
| | | dZ_17658a |
| | | dZ_17713a |
| | | dZ_17730a |
| | | dZ_17780a |
| 303 | Exonuclease | dZ_18135a |
| | 18040/19620 | dZ_18153a |
| | | dZ_18235a |
| | | dZ_18259a |
| | | dZ_18391a |
| | | dZ_18470a |
| | | dZ_18498a |
| | | dZ_18535a |
| | | dZ_18583a |
| | | dZ_18640a |
| | | dZ_18791a |
| | | dZ_18818a |
| | | dZ_18919a |
| | | dZ_18941a |
| | | dZ_18973a |
| | | dZ_19033a |
| | | dZ_19182a |
| | | dZ_19334a |
| | | dZ_19376a |
| | | dZ_19398a |
| 304 | ORF3a 25393/26220 | dZ_15501a |
| | | dZ_25524a |
| | | dZ_25540a |
| | | dZ_25556a |
| | | dZ_25596a |
| | | dZ_25621a |
| | | dZ_25647a |
| | | dZ_25660a |
| | | dZ_25765a |
| | | dZ_25806a |
| | | dZ_25826a |
| | | dZ_25847a |
| | | dZ_25937a |
| | | dZ_25967a |
| | | dZ_26072a |
| | | dZ_26155a |
| 305 | NSP1 266/805 | dZ_341a |
| | | dZ_355a |
| | | dZ_426a |
| | | dZ_468a |
| | | dZ_483a |
| | | dZ_507a |
| | | dZ_558a |
| | | dZ_578a |
| | | dZ_648a |
| | | dZ_688a |
| | | dZ_765a |
| 306 | NSP2 805/2719 | dZ_846a |
| | | dZ_866a |
| | | dZ_910a |
| | | dZ_1015a |
| | | dZ_1051a |
| | | dZ_1080a |
| | | dZ_1168a |
| | | dZ_1210a |
| | | dZ_1243a |
| | | dZ_1308a |
| | | dZ_1338a |
| | | dZ_1367a |
| | | dZ_1431a |
| | | dZ_1475a |
| | | dZ_1599a |
| | | dZ_1719a |
| | | dZ_1759a |
| | | dZ_1796a |
| | | dZ_1846a |
| | | dZ_1940a |
| | | dZ_2020a |
| | | dZ_2127a |
| | | dZ_2167a |
| | | dZ_2244a |
| | | dZ_2276a |
| | | dZ_2376a |
| | | dZ_2426a |
| 307 | NSP3 3027/4791 | dZ_3030a |
| | | dZ_3072a |
| | | dZ_3124a |
| | | dZ_3207a |
| | | dZ_3377a |
| | | dZ_3419a |
| | | dZ_3512a |
| | | dZ_3531a |
| | | dZ_3647a |
| | | dZ_3681a |
| | | dZ_3706a |
| | | dZ_3755a |
| | | dZ_3782a |
| | | dZ_3813a |
| | | dZ_3908a |
| | | dZ_3960a |
| | | dZ_4044a |
| | | dZ_4076a |
| | | dZ_4118a |
| | | dZ_4148a |
| | | dZ_4239a |
| | | dZ_4269a |
| | | dZ_4298a |
| | | dZ_4317a |
| | | dZ_4343a |
| | | dZ_4386a |
| | | dZ_4528a |
| | | dZ_4590a |
| | | dZ_4731a |

TABLE 4
RNA substrates and complementary DNAzymes.

| Sequence | | |
|---|---|---|
| ID | | |
| Number | Name | Complementary RNA Substrates |
| 51 | N_CDCn2- | n2 RNA |
| | 3_M1_1023b | n3 RNA |
| 55 | RCA1 | n1 RNA |
| | | n2 RNA |
| | | n3 RNA |
| 57 | RCA2 | n1 RNA |
| | | n2 RNA |
| | | n3 RNA |
| 59 | RCA3 | nCov_ORF1ab_13470_T7_RNA |
| | | nCov_S_24356_T7_RNA |
| | | nCov_E_26286_T7_RNA |
| 61 | RCA4 | nCov_ORF1ab_13513_T7_RNA |
| | | nCov_S_24526_T7_RNA |
| | | nCov_E_26329_T7_RNA |
| 308 | RCA18b | dZ_14172a digested 5′ RNA |
| | | fragment |
| 309 | RCA196 | dZ_15165a digested 5′ RNA |
| | | fragment |
| 310 | RCA20b | dZ_15202a digested 5′ RNA |
| | | fragment |
| 311 | RCA21b | dZ_15282a digested 5′ RNA |
| | | fragment |
| 312 | RCA22b | dZ_15439a digested 5′ RNA |
| | | fragment |
| 313 | RCA23b | dZ_10491a digested 5′ RNA |
| | | fragment |
| 314 | RCA24b | dZ_507a digested 5′ RNA |
| | | fragment |
| 315 | RCA25b | dZ_11697a digested 5′ RNA |
| | | fragment |
| 316 | RCA26b | dZ_12202a digested 5′ RNA |
| | | fragment |
| 317 | RCA27b | dZ_12290a digested 5′ RNA |
| | | fragment |
| 318 | RCA28b | dZ_12350a digested 5′ RNA |
| | | fragment |
| 319 | RCA29b | dZ_12495a digested 5′ RNA |
| | | fragment |
| 320 | RCA30b | dZ_12618a digested 5′ RNA |
| | | fragment |
| 321 | RCA31b | dZ_20134a digested 5′ RNA |
| | | fragment |
| 322 | RCA32b | dZ_20412a digested 5′ RNA |
| | | fragment |
| 323 | RCA33b | dZ_16583a digested 5′ RNA |
| | | fragment |
| 324 | RCA34b | dZ_16727a digested 5′ RNA |
| | | fragment |
| 325 | RCA35b | dZ_16912a digested 5′ RNA |
| | | fragment |
| 326 | RCA36b | dZ_17522a digested 5′ RNA |
| | | fragment |
| 327 | RCA37b | dZ_18470a digested 5′ RNA |
| | | fragment |
| 328 | RCA38b | dZ_18583a digested 5′ RNA |
| | | fragment |
| 329 | RCA39b | dZ_18973a digested 5′ RNA |
| | | fragment |
| 330 | RCA40b | dZ_19033a digested 5′ RNA |
| | | fragment |
| 331 | RCA41b | dZ_19398a digested 5′ RNA |
| | | fragment |
| 332 | RCA42b | dZ_1308a digested 5′ RNA |
| | | fragment |
| 333 | RCA43b | dZ_1940a digested 5′ RNA |
| | | fragment |
| 334 | RCA44b | dZ_2167a digested 5′ RNA |
| | | fragment |
| 335 | RCA45b | dZ_2426a digested 5′ RNA |
| | | fragment |
| 336 | RCA46b | dZ_3072a digested 5′ RNA |
| | | fragment |
| 337 | RCA47b | dZ_3706a digested 5′ RNA |
| | | fragment |
| 338 | RCA48b | dZ_4076a digested 5′ RNA |
| | | fragment |
| 339 | RCA49b | dZ_4118a digested 5′ RNA |
| | | fragment |
| 340 | RCA50b | dZ_4148a digested 5′ RNA |
| | | fragment |
| 341 | RCA51b | dZ_24086a digested 5′ RNA |
| | | fragment |
| 342 | RCA52b | dZ_21338a digested 5′ RNA |
| | | fragment |

TABLE 5
Oligonucleotides with various lengths of
complementarity to the n1 RNA for CDT
optimization of RNase I activated RCA.

| Sequence | | |
|---|---|---|
| ID Number | Oligo | Sequence (5′-3′) |
| 1 | n1 RNA | GGGAUGUCUGAUAAUGGACCCCAAAAUCAGCGA |
| | | AAUGCACCCCGCAUUACGUUUGGUGGACCCUCA |
| | | GAUUCAACUGGCAGUAACCAGAAUGGAGAACGC |
| | | AGUGGG |
| 55 | RCA1 | CGTAA TGCGG GGTGC |
| | | AGGATCCTGTTTGTAATCAGTTCCTCTTTT |
| | | GGTGT ATTCA |
| 343 | RCA1e05 | CGTAA TGCGG GGTGC ATTTCG |
| | | GGATCCTGTTTGTAATCAGTTCCTCTTTT |
| | | GGTGT ATTCA |
| 344 | RCA1e10 | CGTAA TGCGG GGTGC ATTTCG CTGAT |
| | | GGATCCTGTTTGTAATCAGTTCCTCTTTT |
| | | GGTGT ATTCA |
| 345 | RCA1e15 | CGTAA TGCGG GGTGC ATTTCG CTGAT |
| | | TTTGG |
| | | GGATCCTGTTTGTAATCAGTTCCTCTTTT |
| | | GGTGT ATTCA |
| 346 | RCA1e20 | CGTAA TGCGG GGTGC ATTTCG CTGAT |
| | | TTTGG GGTCC |
| | | GGATCCTGTTTGTAATCAGTTCCTCTTTT |
| | | GGTGT ATTCA |

TABLE 6
DNA oligonucleotides used in the LFD.

| Sequence | | | |
|---|---|---|---|
| ID | | | |
| Number | Name | Sequence | Note |
| 347 | CT | CGTAATGCGGGGTGCTTAAAAAGAC | Underlined part of the |
| | | AGTAGGTACTCATTAGGATCCTGTT | circle is complementary |
| | | TGTAATCAGTTCCTTTTTCTTTTGG | to a part of cleaved |
| | | TGTATTCA | fragment of the N gene |
| | | | (n1 RNA) to start RCA |
| | | | after DNAzyme |
| | | | cleavage |
| 348 | RCAM | TGAATACACCAAAAGAAAAAGGAAC | Monomeric product of |
| | | TGATTACAAACAGGATCCTAATGAG | RCA (complementary to |
| | | TACCTACTGTCTTTTTAAGCACCCC | the circle) |
| | | GCATTACG | |
| 349 | CT-LT | CCGCATTACGTGAATACACCAA | Ligation template to |
| | | | make circle |
| 350 | bDNA | CTAATGAGTACCTACTGTCTAAAAA | It contains an inverted |
| | | AAACTGGATGATCCTATGAACTGA- | dT |
| | | InvdT | |
| 351 | tDNA | TTTTTAGACAGTAGGTACTCATTAG | It contains an inverted |
| | | GATCCTGTTTGTAATC-InvdT | dT |
| 352 | TGNP- | AGACAGTAGGTACTCATTAGTTTTT | DNA for coupling with |
| | DNA | TTTTTSH (SH is thiol) | test gold nanoparticle |
| 353 | TL- | BTTTTTTTTTTTAGTCAGTTCATAG | DNA to print on the test |
| | DNA | GATCATCCAG (B is biotin) | line of LFD |
| 354 | CGNP- | ACCTGGGGGAGTATTGCGGAGGAAG | DNA for coupling with |
| | DNA | GTTTTTTSH (SH is thiol) | control gold |
| | | | nanoparticle |
| 355 | CL- | ACCTTCCTCCGCAATACTCCCCCAG | DNA to print on the |
| | DNA | GTTTTTTB (B is biotin) | control line of LFD |

TABLE 7
DNA oligonucleotides used in the nicking RCA.

| Sequence | | | |
|---|---|---|---|
| ID Number | name | Sequence | Note |
| 356 | Nick-CDT | PGGGTCCATTATCAGACATCCTCAGCT | P is phosphate, |
| | | TTTTAGACAGTAGGTACTCATTAGGAT | underlined italic |
| | | CCTGTTTGTAATCCCTCAGCGCATTTC | are nicking site for |
| | | GCTGATTTTG | Nb.BbvCI |
| 357 | Nick- | ACCTACTGTCTAAAAAGC | Primer for |
| | primer | | initiating RCA |
| 358 | N1Dz.CT1 | GAATCTGAGGGTCCACCAAACGTATCC | Circular template |
| | BA | TCAGCTTCAGTTCATAGGATCATCCAG | for DNAzyme cleave |
| | | AAAAAAAAGACAGTAGGTACTCATTAG | product 1. |
| | | TTCCTCAGCTCA | Underlined italic |
| | | | are nicking site for |
| | | | Nb.BbvCI |
| 359 | N1Dz.CT1 | TGGACCCTCAGATTCTGAGCTGAGGAA | Ligation template |
| | BA.LT | CTAA | for N1Dz.CT1BA |
| 360 | N1Dz.CT2 | CTGCCAGTTGAATCTGAGGGTCTCCTC | Circular template |
| | BA | AGCTTCAGTTCATAGGATCATCCAGAA | for DNAzyme cleave |
| | | AAAAAAGACAGTAGGTACTCATTAGTT | product 1. |
| | | CCTCAGCTCA | Underlined italic |
| | | | are nicking site for |
| | | | Nb.BbvCI |
| 361 | N1Dz.CT2 | AGATTCAACTGGCAGTGAGCTGAGGAA | Ligation template |
| | BA.LT | CTAA | for N1Dz.C21BA |

All publications, patents and patent disclosures are herein incorporated by reference in their entirety to the same extent as if each individual publication, patent or patent disclosure was specifically and individually indicated to be incorporated by reference in its entirety. Where a term in the present disclosure is found to be defined differently in a document incorporated herein by reference, the definition provided herein is to serve as the definition for the term.

### FULL CITATIONS FOR DOCUMENTS REFERRED TO IN THE DISCLOSURE

1: Corman V M, Landt O, Kaiser M, et al. Detection of 2019 novel coronavirus (2019-nCoV) by real-time RT-PCR. Euro surveill. 2020, 25, 23-30.
2: An Update on Abbott's Work on COVID-19 Testing. Abbott Laboratories. Apr. 15, 2020. www.abbott.com/corpnewsroom/product-and-innovation/an-update-on-abbotts-work-on-COVID-19-testing.html.
3: https://www.livescience.com/covid19-coronavirus-tests-false-negatives.html
4: Miura T, Masago Y, Sano D, Omura T. Development of an effective method for recovery of viral genomic RNA from environmental silty sediments for quantitative molecular detection. Appl Environ Microbiol. 2011, 77, 3975-81.
5: Santoro S W, Joyce G F. A general purpose RNA-cleaving DNA enzyme. Proc Natl Acad Sci USA. 1997, 94, 4262-4266.
6: Santoro S W, Joyce G F. Mechanism and utility of an RNA-cleaving DNA enzyme. Biochemistry. 1998, 37, 13330-13342.
7: Liu M, Zhang Q, Li Z, Gu J, Brennan J D, Li Y. Programming a topologically constrained DNA nanostructure into a sensor. Nat Commun. 2016, 7, 12074.
8: Liu M, Zhang Q, Chang D, Gu J, Brennan J D, Li Y. A DNAzyme Feedback Amplification Strategy for Biosensing. Angew Chem Int Ed. 2017, 56, 6142-6146.
9: Kandadai S A, Chiuman W, Li Y. Phosphoester-transfer mechanism of an RNA-cleaving acidic deoxyribozyme revealed by radioactivity tracking and enzymatic digestion. Chem Commun. 2006, 22, 2359-2361.
10: Pan Y, Zhang D, Yang P, Poon L L M, Wang Q. Viral load of SARS-CoV-2 in clinical samples. Lancet Infect Dis. 2020, 20, 411-412.
11: Jahanshahi-Anbuhi S, Pennings K, Leung V, Liu M, Carrasquilla C, Kannan B, Li Y, Pelton R, Brennan J D, Filipe C D. Pullulan encapsulation of labile biomolecules to give stable bioassay tablets. Angew Chem Int Ed. 2014, 53, 6155-6158.
12: Filipe C, Brennan J, Pelton R, Jahanshahi-Anbuhi S, Li Y. Methods of Stabilizing Molecules without Refrigeration using Water Soluble Polymers and Application thereof for Performing Chemical Reactions. US20190178880. Filed on 2016 May 6. Patent Status: Granted/Issued. Year Issued: 2019. https://patentscope.wipo.int/search/en/detail.jsf?docId=US243319619&docAn=16274 616
13: Yurke B, Turberfield A J, Mills A P Jr, Simmel F C, Neumann J L. A DNA-fuelled molecular machine made of DNA. Nature. 2000, 406, 605-608.
14: Zhang D Y, Chen S X, Yin P. Optimizing the specificity of nucleic acid hybridization. Nat Chem. 2012, 4, 208-214.
15: McConnell E M, Cozma I, Morrison D, Li Y. Biosensors Made of Synthetic Functional Nucleic Acids Toward Better Human Health. Anal Chem. 2020, 92, 327-344.
16: Liu M, Zhang W, Zhang Q, Brennan J D, Li Y. Biosensing by Tandem Reactions of Structure Switching, Nucleolytic Digestion, and DNA Amplification of a DNA Assembly. Angew Chem Int Ed. 2015, 54, 9637-9641.
17: Li Y, Brennan J, Liu M. Biosensor comprising tandem reactions of structure switching, nucleolytic digestion, and amplification of nucleic acid assembly. PCT/CA2016/05073, filed on 2016 Jun. 16; https://patentscope.wipo.int/search/en/detail.jsf?docId=WO2016205940
18: Shevelev I V, Hubscher U. The 3′ 5′ exonucleases. Nat Rev Mol Cell Biol. 2002, 3, 364-376.

1. A recognition moiety comprising a catalytic nucleic acid,
wherein the recognition moiety recognizes a target nucleic acid and cleaves the target nucleic acid upon contact to produce a cleavage fragment that acts as a primer for rolling circle amplification (RCA) to generate single-stranded nucleic acid molecules, and
wherein the target nucleic acid is from SARS-CoV-2.

2. (canceled)

3. The recognition moiety of claim 1, wherein the catalytic nucleic acid comprises a nucleic acid molecule having a sequence as set forth in at least one of SEQ ID NO: 17-19, 21, 22, 66, 80, 81, 91, 92, 96, 109, 112, 114, 123, 130, 139, 145, 151, 160, 179, 182, 188, 203, 215, 230, 236, 249, 259, 262, 266, 268, and 284.

4. (canceled)

5. The recognition moiety of claim 1, wherein the catalytic nucleic acid comprises a nucleic acid molecule having a sequence as set forth in at least one of SEQ ID NO: 80, 123, 130, 203, and 268.

6. The recognition moiety of claim 1, wherein the target nucleic acid has a sequence as set forth in at least one of SEQ ID NO: 98, 298, 299, 304, and 307.

7. (canceled)

8. The recognition moiety of claim 1, wherein the catalytic nucleic acid comprises a nucleic acid molecule having a sequence as set forth in SEQ ID NO: 80, and the target nucleic acid has a sequence as set forth in SEQ ID NO: 98.

9. A biosensor for detecting a target nucleic acid comprising:
a) a recognition moiety comprising a catalytic nucleic acid;
b) a polynucleotide kinase or phosphatase; and
c) reagents for performing rolling circle amplification (RCA);
wherein the recognition moiety cleaves the target nucleic acid to produce a cleavage fragment and the polynucleotide kinase or phosphatase removes cyclic phosphate from the cleavage fragment, producing a dephosphorylated cleavage fragment that acts as a primer for RCA to generate single-stranded nucleic acid molecules.

10. (canceled)

11. The biosensor of claim 9, wherein the catalytic nucleic acid acts as a circular DNA template for performing rolling circle amplification (RCA) or the reagents for performing RCA further comprise a circular DNA template.

12. The biosensor of claim 9, wherein the recognition moiety comprises a nuclease.

13. The biosensor of claim 12, wherein the nuclease is a ribonuclease, optionally, RNase I.

14-17. (canceled)

18. The biosensor of claim 9, further comprising lysis agents.

19. (canceled)

20. The biosensor of claim 9, further comprising a reporter moiety comprising a detectable label that generates a fluorescent, colorimetric, electrochemical, surface plasmon resonance, spectroscopic, or radioactive signal.

21-25. (canceled)

26. The biosensor of claim 9, wherein the recognition moiety comprises nucleic acid molecule having a sequence as set forth in at least one of SEQ ID NO: 17-19, 21, 22, 66, 80, 81, 91, 92, 96, 109, 112, 114, 123, 130, 139, 145, 151, 160, 179, 182, 188, 203, 215, 230, 236, 249, 259, 262, 266, 268, and 284.

27. (canceled)

28. The biosensor of claim 9, wherein the recognition moiety comprises a nucleic acid molecule having a sequence as set forth in at least one of SEQ ID NO: 80, 123, 130, 203, and 268.

29. (canceled)

30. The biosensor of claim 9, wherein the target nucleic acid has a sequence as set forth in at least one of SEQ ID NO: 98, 298, 299, 304, and 307.

31. (canceled)

32. The biosensor of claim 9, wherein the recognition moiety comprises a nucleic acid molecule having a sequence as set forth in SEQ ID NO: 80, and the target nucleic acid has a sequence as set forth in SEQ ID NO: 98.

33. The biosensor of claim 9, further comprising a lateral flow device for detecting the target nucleic acid.

34-54. (canceled)

55. A method for detecting the presence of a target nucleic acid in a sample, comprising:
a) contacting the sample with a recognition moiety, wherein the recognition moiety cleaves the target nucleic acid to produce a cleavage fragment;
b) removing cyclic phosphate from the cleavage fragment with a polynucleotide kinase or phosphatase;
c) performing rolling circle amplification (RCA) on the cleavage fragment under conditions to generate single-stranded nucleic acid molecules; and
d) detecting the single-stranded nucleic acid molecules generated in c);
wherein detection of the single-stranded nucleic acid molecules in d) indicates presence of the target nucleic acid in the sample.

56. The method of claim 55, further comprising contacting the sample with lysis agents prior to contacting the sample with the recognition moiety.

57. The method of claim 55, wherein detection of the single-stranded nucleic acid molecules is indicated by a fluorescent, colorimetric, electrochemical, surface plasmon resonance, spectroscopic, or radioactive signal.

58-59. (canceled)

60. The method of claim 55, wherein detection of the single-stranded nucleic acid molecules comprises:
a) providing a first single-stranded oligonucleotide partially hybridized to a second single-stranded oligonucleotide prior to RCA;
b) preferentially hybridizing the second single-stranded oligonucleotide to repeating segments of the single-stranded nucleic acid molecules produced from the RCA, displacing the first single-stranded oligonucleotide;
c) hybridizing a first domain of the first single-stranded oligonucleotide to a reporter moiety, wherein the reporter moiety is disposed near a first end of lateral flow test strip;
d) flowing the reporter moiety hybridized to the first domain of the first single-stranded oligonucleotide from a first end of the lateral flow test strip towards a second end of the lateral flow test strip; and
e) hybridizing a second domain of the first single-stranded oligonucleotide to a capture probe, wherein the capture probe is immobilized on the lateral flow test strip in a visualization area.

61-65. (canceled)


SEQUENCE LISTING (361 sequences)

SEQ ID NO: 1 - RNA, 105 nt, SARS-CoV-2
gggaugucugauaauggaccccaaaaucagcgaaaugcaccccgcauuacguuugguggacccucagauucaacuggcaguaaccagaauggagaacgcaguggg

SEQ ID NO: 2 - RNA, 103 nt, SARS-CoV-2
ggguauggguugcaacugagggagccuugaauacaccaaaagaucacauuggcacccgcaauccugcuaacaaugcugcaaucgugcuacaacuuccucaagg

SEQ ID NO: 3 - RNA, 103 nt, SARS-CoV-2
gggccaggaacuaaucagacaaggaacugauuacaaacauuggccgcaaauugcacaauuugcccccagcgcuucagcguucuucggaaugucgcgcauuggc

SEQ ID NO: 4 - RNA, 66 nt, SARS-CoV-2
ggguuugcgguguaagugcagcccgucuuacaccgugcggcacaggcacuaguacugaugucguau

SEQ ID NO: 5 - RNA, 66 nt, SARS-CoV-2
gggcacuaguacugaugucguauacagggcuuuugacaucuacaaugauaaaguagcugguuuugc

SEQ ID NO: 6 - RNA, 66 nt, SARS-CoV-2
gggcaaaauucaagacucacuuucuuccacagcaagugcacuuggaaaacuucaagauguggucaa

SEQ ID NO: 7 - RNA, 66 nt, SARS-CoV-2
gggcugaagugcaaauugauagguugaucacaggcagacuucaaaguuugcagacauaugugacuc

SEQ ID NO: 8 - RNA, 68 nt, SARS-CoV-2
ggguaauagcguacuucuuuuucuugcuuucgugguauucuugcuaguuacacuagccauccuuacug

SEQ ID NO: 9 - RNA, 67 nt, SARS-CoV-2
ggguuacacuagccauccuuacugcgcuucgauugugugcguacugcugcaauauuguuaacgugag

SEQ ID NO: 10 - DNA, 30 bp, Artificial Sequence
  Synthetic Construct
ccaccaaaggctagctacaacgagtaatgc

SEQ ID NO: 11 - DNA, 34 bp, Artificial Sequence
  Synthetic Construct
gggtccaccaaaggctagctacaacgagtaatgc

SEQ ID NO: 12 - DNA, 36 bp, Artificial Sequence
  Synthetic Construct
agggtccaccaaaggctagctacaacgagtaatgcg

SEQ ID NO: 13 - DNA, 37 bp, Artificial Sequence
  Synthetic Construct
gagggtccaccaaaggctagctacaacgagtaatgcg

SEQ ID NO: 14 - DNA, 39 bp, Artificial Sequence
  Synthetic Construct
ctgagggtccaccaaaggctagctacaacgagtaatgcg

SEQ ID NO: 15 - DNA, 44 bp, Artificial Sequence
  Synthetic Construct
tgaatctgagggtccaccaaaggctagctacaacgagtaatgcg

SEQ ID NO: 16 - DNA, 16 bp, SARS-CoV-2
tgcaccccgcattacg

SEQ ID NO: 17 - DNA, 30 bp, Artificial Sequence
  Synthetic Construct
tctggttaggctagctacaacgatgccagt

SEQ ID NO: 18 - DNA, 35 bp, Artificial Sequence
  Synthetic Construct
tccattctggttaggctagctacaacgatgccagt

SEQ ID NO: 19 - DNA, 39 bp, Artificial Sequence
  Synthetic Construct
ttctccattctggttaggctagctacaacgatgccagtt

SEQ ID NO: 20 - DNA, 16 bp, SARS-CoV-2
cagattcaactggcag

SEQ ID NO: 21 - DNA, 30 bp, Artificial Sequence
  Synthetic Construct
caatgtgaggctagctacaacgacttttgg

SEQ ID NO: 22 - DNA, 39 bp, Artificial Sequence
  Synthetic Construct
gcgggtgccaatgtgaggctagctacaacgacttttggt

SEQ ID NO: 23 - DNA, 16 bp, SARS-CoV-2
tgaatacaccaaaaga

SEQ ID NO: 24 - DNA, 30 bp, Artificial Sequence
  Synthetic Construct
tagcaggaggctagctacaacgatgcgggt

SEQ ID NO: 25 - DNA, 39 bp, Artificial Sequence
  Synthetic Construct
agcattgttagcaggaggctagctacaacgatgcgggtg

SEQ ID NO: 26 - DNA, 16 bp, SARS-CoV-2
acattggcacccgcaa

SEQ ID NO: 27 - DNA, 30 bp, Artificial Sequence
  Synthetic Construct
gcggccaaggctagctacaacgagtttgta

SEQ ID NO: 28 - DNA, 39 bp, Artificial Sequence
  Synthetic Construct
tgcaatttgcggccaaggctagctacaacgagtttgtaa

SEQ ID NO: 29 - DNA, 16 bp, SARS-CoV-2
gaactgattacaaaca

SEQ ID NO: 30 - DNA, 30 bp, Artificial Sequence
  Synthetic Construct
ccgaagaaggctagctacaacgagctgaag

SEQ ID NO: 31 - DNA, 39 bp, Artificial Sequence
  Synthetic Construct
gcgacattccgaagaaggctagctacaacgagctgaagc

SEQ ID NO: 32 - DNA, 16 bp, SARS-CoV-2
ccccagcgcttcagcg

SEQ ID NO: 33 - DNA, 30 bp, Artificial Sequence
  Synthetic Construct
gtgtaagaggctagctacaacgagggctgc

SEQ ID NO: 34 - DNA, 39 bp, Artificial Sequence
  Synthetic Construct
gccgcacggtgtaagaggctagctacaacgagggctgca

SEQ ID NO: 35 - DNA, 16 bp, SARS-CoV-2
gtgtaagtgcagcccg

SEQ ID NO: 36 - DNA, 30 bp, Artificial Sequence
  Synthetic Construct
attgtagaggctagctacaacgagtcaaaa

SEQ ID NO: 37 - DNA, 39 bp, Artificial Sequence
  Synthetic Construct
actttatcattgtagaggctagctacaacgagtcaaaag

SEQ ID NO: 38 - DNA, 16 bp, SARS-CoV-2
tacagggcttttgaca

SEQ ID NO: 39 - DNA, 30 bp, Artificial Sequence
  Synthetic Construct
caagtgcaggctagctacaacgattgctgt

SEQ ID NO: 40 - DNA, 39 bp, Artificial Sequence
  Synthetic Construct
aagttttccaagtgcaggctagctacaacgattgctgtg

SEQ ID NO: 41 - DNA, 16 bp, SARS-CoV-2
tttcttccacagcaag

SEQ ID NO: 42 - DNA, 30 bp, Artificial Sequence
  Synthetic Constuct
gcctgtgaggctagctacaacgacaaccta

SEQ ID NO: 43 - DNA, 39 bp, Artificial Sequence
  Synthetic Construct
tgaagtctgcctgtgaggctagctacaacgacaacctat

SEQ ID NO: 44 - DNA, 16 bp, SARS-CoV-2
caaattgataggttga

SEQ ID NO: 45 - DNA, 30 bp, SARS-CoV-2
agcaagaaggctagctacaacgaaccacga

SEQ ID NO: 46 - DNA, 39 bp, SARS-CoV-2
gtgtaactagcaagaaggctagctacaacgaaccacgaa

SEQ ID NO: 47 - DNA, 16 bp, SARS-CoV-2
tcttgctttcgtggta

SEQ ID NO: 48 - DNA, 30 bp, Artificial Sequence
  Synthetic Construst
gcacacaaggctagctacaacgacgaagcg

SEQ ID NO: 49 - DNA, 39 bp, Artificial Sequence
  Synthetic Construct
agcagtacgcacacaaggctagctacaacgacgaagcgc

SEQ ID NO: 50 - DNA, 16 bp, SARS-CoV-2
ccttactgcgcttcga

SEQ ID NO: 51 - DNA, 82 bp, Artificial Sequence
  Synthetic Construct
caatgtgaggctagctacaacgtcttttggtgtattcaggatccgcggccaaggctagctacaacgtgtttgtaatcagttc

SEQ ID NO: 52 - DNA, 20 bp, Artificial Sequence
  Synthetic Construct
cctcacattggaactgatta

SEQ ID NO: 53 - DNA, 16 bp, Artificial Sequence
  Synthetic Construct
tgaatacaccaaaaga

SEQ ID NO: 54 - DNA, 16 bp, Artificial Sequence
  Synthetic Construct
gaactgattacaaaca

SEQ ID NO: 55 - DNA, 55 bp, Artificial Sequence
  Synthetic Construct
cgtaatgcggggtgcaggatcctgtttgtaatcagttcctcttttggtgtattca

SEQ ID NO: 56 - DNA, 20 bp, Artificial Sequence
  Synthetic Construct
ccgcattacgtgaatacacc

SEQ ID NO: 57 - DNA, 54 bp, Artificial Sequence
  Synthetic Construct
ctgccagttgaatctgggatccttgcgggtgccaatgtcgctgaagcgctgggg

SEQ ID NO: 58 - DNA, 20 bp, Artificial Sequence
  Synthetic Construct
caactggcagccccagcgct

SEQ ID NO: 59 - DNA, 54 bp, Artificial Sequence
  Synthetic Construct
cgggctgcacttacacggatcccttgctgtggaagaaataccacgaaagcaaga

SEQ ID NO: 60 - DNA, 20 bp, Artificial Sequence
  Synthetic Construct
gtgcagcccgtcttgctttc

SEQ ID NO: 61 - DNA, 54 bp, Artificial Sequence
  Synthetic Construct
tgtcaaaagccctgtaggatcctcaacctatcaatttgtcgaagcgcagtaagg

SEQ ID NO: 62 - DNA, 20 bp, Artificial Sequence
  Synthetic Construct
gcttttgacaccttactgcg

SEQ ID NO: 63 - DNA, 39 bp, Artificial Sequence
  Synthetic Construct
gtgatcttttggtgtaggctagctacaacgatcaaggct

SEQ ID NO: 64 - DNA, 39 bp, Artificial Sequence
  Synthetic Construct
tagcacgattgcagcaggctagctacaacgatgttagca

SEQ ID NO: 65 - DNA, 39 bp, Artificial Sequence
  Synthetic Construct
agaagccttttggcaaggctagctacaacgagttgttcc

SEQ ID NO: 66 - DNA, 39 bp, Artificial Sequence
  Synthetic Construct
agttgaatttcttgaaggctagctacaacgatgttgcga

SEQ ID NO: 67 - DNA, 39 bp, Artificial Sequence
  Synthetic Construct
atggaaccaagtaacaggctagctacaacgatggaaaag

SEQ ID NO: 68 - DNA, 39 bp, Artificial Sequence
  Synthetic Construct
attggtcccagagacaggctagctacaacgagtatagca

SEQ ID NO: 69 - DNA, 39 bp, Artificial Sequence
  Synthetic Construct
caaaaatggatcattaggctagctacaacgaaaaattga

SEQ ID NO: 70 - DNA, 39 bp, Artificial Sequence
  Synthetic Construct
agaatatattttaaaaggctagctacaacgaaaccatca

SEQ ID NO: 71 - DNA, 39 bp, Artificial Sequence
  Synthetic Construct
cttcctgttccaagcaggctagctacaacgaaaacagat

SEQ ID NO: 72 - DNA, 39 bp, Artificial Sequence
  Synthetic Construct
ttaaagcacggtttaaggctagctacaacgatgtgtaca

SEQ ID NO: 73 - DNA, 39 bp, Artificial Sequence
  Synthetic Construct
acagtgcagaagtgtaggctagctacaacgatgagcaat

SEQ ID NO: 74 - DNA, 39 bp, Artificial Sequence
  Synthetic Construct
tgaaattgcaccaaaaggctagctacaacgatggagcta

SEQ ID NO: 75 - DNA, 39 bp, Artificial Sequence
  Synthetic Construct
gactgagggaaggacaggctagctacaacgaaagatgat

SEQ ID NO: 76 - DNA, 39 bp, Artificial Sequence
  Synthetic Construct
tcaatttctttttgaaggctagctacaacgagtttacaa

SEQ ID NO: 77 - DNA, 39 bp, Artificial Sequence
  Synthetic Construct
ctacagcaactggtcaggctagctacaacgaacagcaaa

SEQ ID NO: 78 - DNA, 39 bp, Artificial Sequence
  Synthetic Construct
tgtcaaaagccctgtaggctagctacaacgaacgacatc

SEQ ID NO: 79 - DNA, 39 bp, Artificial Sequence
  Synthetic Construct
atcaattaaattgtcaggctagctacaacgacttcgtcc

SEQ ID NO: 80 - DNA, 39 bp, Artificial Sequence
  Synthetic Construct
aagtcatgtttagcaaggctagctacaacgaagctggac

SEQ ID NO: 81 - DNA, 39 bp, Artificial Sequence
  Synthetic Construct
ccctggtcaaggttaaggctagctacaacgaataggcat

SEQ ID NO: 82 - DNA, 39 bp, Artificial Sequence
  Synthetic Construct
ccagaagcagcgtgcaggctagctacaacgaagcagggt

SEQ ID NO: 83 - DNA, 39 bp, Artificial Sequence
  Synthetic Construct
gttgtctgatatcacaggctagctacaacgaattgttgg

SEQ ID NO: 84 - DNA, 39 bp, Artificial Sequence
  Synthetic Construct
actcattgaatcataaggctagctacaacgaaaagtcta

SEQ ID NO: 85 - DNA, 39 bp, Artificial Sequence
  Synthetic Construct
gacattacgttttgtaggctagctacaacgaatgcgaaa

SEQ ID NO: 86 - DNA, 39 bp, Artificial Sequence
  Synthetic Construct
cggctattgatttcaaggctagctacaacgaaatttttg

SEQ ID NO: 87 - DNA, 39 bp, Artificial Sequence
  Synthetic Construct
ttgcttgttccaattaggctagctacaacgatacagtag

SEQ ID NO: 88 - DNA, 39 bp, Artificial Sequence
  Synthetic Construct
ggataatcccaacccaggctagctacaacgaaaggtgag

SEQ ID NO: 89 - DNA, 39 bp, Artificial Sequence
  Synthetic Construct
aaaaacactattagcaggctagctacaacgaaagcagtt

SEQ ID NO: 90 - DNA, 39 bp, Artificial Sequence
  Synthetic Construct
gaaccgccacacatgaggctagctacaacgacatttcac

SEQ ID NO: 91 - DNA, 39 bp, Artificial Sequence
  Synthetic Construct
tcagagagtatcatcaggctagctacaacgatgagaaat

SEQ ID NO: 92 - DNA, 39 bp, Artificial Sequence
  Synthetic Construct
ctgggtaaggaaggtaggctagctacaacgaacataatc

SEQ ID NO: 93 - DNA, 39 bp, Artificial Sequence
  Synthetic Construct
aggaaaattaacttaaggctagctacaacgatatataca

SEQ ID NO: 94 - DNA, 39 bp, Artificial Sequence
  Synthetic Construct
taaacagcagcaagcaggctagctacaacgaaaaacaag

SEQ ID NO: 95 - DNA, 39 bp, Artificial Sequence
  Synthetic Construct
ggcacgttgagaagaaggctagctacaacgagttagttt

SEQ ID NO: 96 - DNA, 39 bp, Artificial Sequence
  Synthetic Construct
aatggtctgtgtttaaggctagctacaacgattatagtt

SEQ ID NO: 97 - RNA, 1263 nt, SARS-CoV-2
gggaugucugauaauggaccccaaaaucagcgaaaugcaccccgcauuacguuugguggacccucagauucaacuggcaguaaccagaauggagaacgcaguggggcgcgaucaaaacaacgucggccccaagguuuacccaauaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaa

SEQ ID NO: 98 - RNA, 1208 nt, SARS-CoV-2
ggguuugcgguguaagugcagcccgucuuacaccgugcggcacaggcacuaguacugaugucguauacagggcuuuugacaucuacaaugauaaaguagcugguuuugcuaaauuccuaaaaacuaauuguugucgcuuccaagaaaaggacgaagaugacaauuuaauugauucuuacuuuguaguuaagagacacacuuucucuaacuaccaacaugaagaaacaauuuauaauuuacuuaaggauuguccagcuguugcuaaacaugacuucuuuaaguuuagaauagacggugacaugguaccacauauaucacgucaacgucuuacuaaauacacaauggcagaccucgucuaugcuuuaaggcauuuugaugaagguaauugugacacauuaaaagaaauacuugucacauacaauuguugugaugaugauuauuucaauaaaaaggacugguaugauuuuguagaaaacccagauauauuacgcguauacgccaacuuaggugaacguguacgccaagcuuuguuaaaaacaguacaauucugugaugccaugcgaaaugcugguauuguugguguacugacauuagauaaucaagaucucaaugguaacugguaugauuucggugauuucauacaaaccacgccagguaguggaguuccuguuguagauucuuauuauucauuguuaaugccuauauuaaccuugaccagggcuuuaacugcagagucacauguugacacugacuuaacaaagccuuacauuaagugggauuuguuaaaauaugacuucacggaagagagguuaaaacucuuugaccguuauuuuaaauauugggaucagacauaccacccaaauuguguuaacuguuuggaugacagaugcauucugcauugugcaaacuuuaauguuuuauucucuacaguguucccaccuacaaguuuuggaccacuagugagaaaaauauuuguugaugguguuccauuuguaguuucaacuggauaccacuucagagagcuagguguuguacauaaucaggauguaaacuuacauagcucuagacuuaguuuuaaggaauuacuuguguaugcugcugacccugcuaugcacgcugcuucugguaaucuauuacuagauaaacgcacuacgugcuuuucaguagcugcacuuacuaacaauguugcuuuucaaacugucaaaccc

SEQ ID NO: 99 - RNA, 1433 nt, SARS-CoV-2
gggcucaggaugguaaugcugcuaucagcgauuaugacuacuaucguuauaaucuaccaacaaugugugauaucagacaacuacuauuuguaguugaaguuguugauaaguacuuugauuguuacgaugguggcuguauuaaugcuaaccaagucaucgucaacaaccuagacaaaucagcugguuuuccauuuaauaaaugggguaaggcuagacuuuauuaugauucaaugaguuaugaggaucaagaugcacuuuucgcauauacaaaacguaaugucaucccuacuauaacucaaaugaaucuuaaguaugccauuagugcaaagaauagagcucgcaccguagcuggugucucuaucuguaguacuaugaccaauagacaguuucaucaaaaauuauugaaaucaauagccgccacuagaggagcuacuguaguaauuggaacaagcaaauucuauggugguuggcacaacauguuaaaaacuguuuauagugauguagaaaacccucaccuuauggguugggauuauccuaaaugugauagagccaugccuaacaugcuuagaauuauggccucacuuguucuugcucgcaaacauacaacguguuguagcuugucacaccguuucuauagauuagcuaaugagugugcucaaguauugagugaaauggucauguguggcgguucacuauauguuaaaccagguggaaccucaucaggagaugccacaacugcuuaugcuaauaguguuuuuaacauuugucaagcugucacggccaauguuaaugcacuuuuaucuacugaugguaacaaaauugccgauaaguauguccgcaauuuacaacacagacuuuaugagugucucuauagaaauagagauguugacacagacuuugugaaugaguuuuacgcauauuugcguaaacauuucucaaugaugauacucucugacgaugcuguuguguguuucaauagcacuuaugcaucucaaggucuaguggcuagcauaaagaacuuuaagucaguucuuuauuaucaaaacaauguuuuuaugucugaagcaaaauguuggacugagacugaccuuacuaaaggaccucaugaauuuugcucucaacauacaaugcuaguuaaacagggugaugauuauguguaccuuccuuacccagauccaucaagaauccuaggggccggcuguuuuguagaugauaucguaaaaacagaugguacacuuaugauugaacgguucgugucuuuagcuauagaugcuuacccacuuacuaaacauccuaaucaggaguaugcugaugucuuucauuuguacuuacaauacauaagaaagcuacaugaugaguuaacaggacacauguuagacauguauucuguuaugcuuacuaaugauaacacuucaagguauugggaaccugag

SEQ ID NO: 100 - RNA, 769 nt, SARS-CoV-2
ggguuucacacgugguguuuauuacccugacaaaguuuucagauccucaguuuuacauucaacucaggacuuguucuuaccuuucuuuuccaauguuacuugguuccaugcuauacaugucucugggaccaaugguacuaagagguuugauaacccuguccuaccauuuaaugaugguguuuauuuugcuuccacugagaagucuaacauaauaagaggcuggauuuuugguacuacuuuagauucgaagacccagucccuacuuauuguuaauaacgcuacuaauguuguuauuaaagucugugaauuucaauuuuguaaugauccauuuuuggguguuuauuaccacaaaaacaacaaaaguuggauggaaagugaguucagaguuuauucuagugcgaauaauugcacuuuugaauaugucucucagccuuuucuuauggaccuugaaggaaaacaggguaauuucaaaaaucuuagggaauuuguguuuaagaauauugaugguuauuuuaaaauauauucuaagcacacgccuauuaauuuagugcgugaucucccucaggguuuuucggcuuuagaaccauugguagauuugccaauagguauuaacaucacuagguuucaaacuuuacuugcuuuacauagaaguuauuugacuccuggugauucuucuucagguuggacagcuggugcugcagcuuauuauguggguuaucuucaaccuaggacuuuucuauuaaaauauaaugaaaauggaaccauuaca

SEQ ID NO: 101 - RNA, 704 nt, SARS-CoV-2
gggaugcuguagacugugcacuugacccucucucagaaacaaaguguacguugaaauccuucacuguagaaaaaggaaucuaucaaacuucuaacuuuagaguccaaccaacagaaucuauuguuagauuuccuaauauuacaaacuugugcccuuuuggugaaguuuuuaacgccaccagauuugcaucuguuuaugcuuggaacaggaagagaaucagcaacuguguugcugauuauucuguccuauauaauuccgcaucauuuuccacuuuuaaguguuauggagugucuccuacuaaauuaaaugaucucugcuuuacuaaugucuaugcagauucauuuguaauuagaggugaugaagucagacaaaucgcuccagggcaaacuggaaagauugcugauuauaauuauaaauuaccagaugauuuuacaggcugcguuauagcuuggaauucuaacaaucuugauucuaagguuggugguaauuauaauuaccuguauagauuguuuaggaagucuaaucucaaaccuuuugagagagauauuucaacugaaaucuaucaggccgguagcacaccuuguaaugguguugaagguuuuaauuguuacuuuccuuuacaaucauaugguuuccaacccacuaaugguguugguuaccaaccauacagaguaguaguacuuucuuuugaacuucuacaugca

SEQ ID NO: 102 - RNA, 478 nt, SARS-CoV-2
gggugcagaucaacuuacuccuacuuggcguguuuauucuacagguucuaauguuuuucaaacacgugcaggcuguuuaauaggggcugaacaugucaacaacucauaugagugugacauacccauuggugcagguauaugcgcuaguuaucagacucagacuaauucuccucggcgggcacguaguguagcuagucaauccaucauugccuacacuaugucacuuggugcagaaaauucaguugcuuacucuaauaacucuauugccauacccacaaauuuuacuauuaguguuaccacagaaauucuaccagugucuaugaccaagacaucaguagauuguacaauguacauuuguggugauucaacugaaugcagcaaucuuuuguugcaauauggcaguuuuuguacacaauuaaaccgugcuuuaacuggaauagcuguugaacaagacaaaaacacccaagaaguuuuugca

SEQ ID NO: 103 - RNA, 566 nt, SARS-CoV-2
ggguuuccccauuugugcacaaaaguuuaacggccuuacuguuuugccaccuuugcucacagaugaaaugauugcucaauacacuucugcacuguuagcggguacaaucacuucugguuggaccuuuggugcaggugcugcauuacaaauaccauuugcuaugcaaauggcuuauagguuuaaugguauuggaguuacacagaauguucucuaugagaaccaaaaauugauugccaaccaauuuaauagugcuauuggcaaaauucaagacucacuuucuuccacagcaagugcacuuggaaaacuucaagauguggucaaccaaaaugcacaagcuuuaaacacgcuuguuaaacaacuuagcuccaauuuuggugcaauuucaaguguuuuaaaugauauccuuucacgucuugacaaaguugaggcugaagugcaaauugauagguugaucacaggcagacuucaaaguuugcagacauaugugacucaacaauuaauuagagcugcagaaaucagagcuucugcuaaucuugcugcuacuaaaaugucagaguguguacuug

SEQ ID NO: 104 - RNA, 682 nt, SARS-CoV-2
gggcaaaaaucaaaaagaguugauuuuuguggaaagggcuaucaucuuauguccuucccucagucagcaccucaugguguagucuucuugcaugugacuuaugucccugcacaagaaaagaacuucacaacugcuccugccauuugucaugauggaaaagcacacuuuccucgugaaggugucuuuguuucaaauggcacacacugguuuguaacacaaaggaauuuuuaugaaccacaaaucauuacuacagacaacacauuugugucugguaacugugauguuguaauaggaauugucaacaacacaguuuaugauccuuugcaaccugaauuagacucauucaaggaggaguuagauaaauauuuuaagaaucauacaucaccagauguugauuuaggugacaucucuggcauuaaugcuucaguuguaaacauucaaaaagaaauugaccgccucaaugagguugccaagaauuuaaaugaaucucucaucgaucuccaagaacuuggaaaguaugagcaguauauaaaauggccaugguacauuuggcuagguuuuauagcuggcuugauugccauaguaauggugacaauuaugcuuugcuguaugaccaguugcuguaguugucucaagggcuguuguucuuguggauccugcugcaaauuugaugaagacgacu

SEQ ID NO: 105 - DNA, 39 bp, Artificial Sequence
  Synthetic Construct
tacttgtaccatacaaggctagctacaacgacctcaact

SEQ ID NO: 106 - DNA, 39 bp, Artificial Sequence
  Synthetic Construct
gtcatcaagccaaagaggctagctacaacgacgttaagt

SEQ ID NO: 107 - DNA, 39 bp, Artificial Sequence
  Synthetic Construct
agaggtgcagatcacaggctagctacaacgagtcttgga

SEQ ID NO: 108 - DNA, 39 bp, Artificial Sequence
  Synthetic Construct
acattaccagcctgtaggctagctacaacgacaagaaat

SEQ ID NO: 109 - DNA, 39 bp, Artificial Sequence
  Synthetic Construct
ggattggctgtatcaaggctagctacaacgacttaagct

SEQ ID NO: 110 - DNA, 39 bp, Artificial Sequence
  Synthetic Construct
cttaggtgtcttaggaggctagctacaacgatggctgta

SEQ ID NO: 111 - DNA, 39 bp, Artificial Sequence
  Synthetic Construct
gtgaaattgggcctcaggctagctacaacgaagcacatt

SEQ ID NO: 112 - DNA, 39 bp, Artificial Sequence
  Synthetic Construct
gttaaaaccaacactaggctagctacaacgacacatgaa

SEQ ID NO: 113 - DNA, 39 bp, Artificial Sequence
  Synthetic Construct
gtcaacaaaaggtccaggctagctacaacgaaaaagtta

SEQ ID NO: 114 - DNA, 39 bp, Artificial Sequence
  Synthetic Construct
gaaagaggtcctagtaggctagctacaacgagtcaacat

SEQ ID NO: 115 - DNA, 39 bp, Artificial Sequence
  Synthetic Construct
aaaagaacaaagaccaggctagctacaacgatgagtact

SEQ ID NO: 116 - DNA, 39 bp, Artificial Sequence
  Synthetic Construct
taaaaaggcattttcaggctagctacaacgaacaaaaaa

SEQ ID NO: 117 - DNA, 39 bp, Artificial Sequence
  Synthetic Construct
atagcaataatacccaggctagctacaacgaagcaaaag

SEQ ID NO: 118 - DNA, 39 bp, Artificial Sequence
  Synthetic Construct
aggcatatagaccataggctagctacaacgataaaataa

SEQ ID NO: 119 - DNA, 39 bp, Artificial Sequence
  Synthetic Construct
aaactagtatcaaccaggctagctacaacgaatccaacc

SEQ ID NO: 120 - DNA, 39 bp, Artificial Sequence
  Synthetic Construct
cttgctgtcataaggaggctagctacaacgatagtaaca

SEQ ID NO: 121 - DNA, 39 bp, Artificial Sequence
  Synthetic Construct
gacagttgtaactacaggctagctacaacgactgagtag

SEQ ID NO: 122 - DNA, 39 bp, Artificial Sequence
  Synthetic Construct
tacctctggccaaaaaggctagctacaacgaatgacagt

SEQ ID NO: 123 - DNA, 39 bp, Artificial Sequence
  Synthetic Construct
ccagttatgaagaaaaggctagctacaacgaagggcaat

SEQ ID NO: 124 - DNA, 39 bp, Artificial Sequence
  Synthetic Construct
aaaatagcctaagaaaggctagctacaacgaaataaact

SEQ ID NO: 125 - DNA, 39 bp, Artificial Sequence
  Synthetic Construct
agaaactaagtaatcaggctagctacaacgaaaacacca

SEQ ID NO: 126 - DNA, 39 bp, Artificial Sequence
  Synthetic Construct
tccctgtgaattcataggctagctacaacgaatctaaac

SEQ ID NO: 127 - DNA, 39 bp, Artificial Sequence
  Synthetic Construct
agcaacagcctgctcaggctagctacaacgaaagcttct

SEQ ID NO: 128 - DNA, 39 bp, Artificial Sequence
  Synthetic Construct
aacttcagaatcaccaggctagctacaacgatagcaaca

SEQ ID NO: 129 - DNA, 39 bp, Artificial Sequence
  Synthetic Construct
tcaaagacttcttcaaggctagctacaacgatttttaag

SEQ ID NO: 130 - DNA, 39 bp, Artificial Sequence
  Synthetic Construct
catcttttccaacttaggctagctacaacgagttgcatg

SEQ ID NO: 131 - DNA, 39 bp, Artificial Sequence
  Synthetic Construct
ttatacatttgggtcaggctagctacaacgaagcttgat

SEQ ID NO: 132 - DNA, 39 bp, Artificial Sequence
  Synthetic Construct
ctagcctgtttatacaggctagctacaacgattgggtca

SEQ ID NO: 133 - DNA, 39 bp, Artificial Sequence
  Synthetic Construct
aaaagcattgtctgcaggctagctacaacgaagcactag

SEQ ID NO: 134 - DNA, 39 bp, Artificial Sequence
  Synthetic Construct
agcatagtgaaaagcaggctagctacaacgatgtctgca

SEQ ID NO: 135 - DNA, 39 bp, Artificial Sequence
  Synthetic Construct
atttttatatgtgttaggctagctacaacgaagtctggt

SEQ ID NO: 136 - DNA, 39 bp, Artificial Sequence
  Synthetic Construct
tctacaacctgttggaggctagctacaacgattcccaca

SEQ ID NO: 137 - DNA, 39 bp, Artificial Sequence
  Synthetic Construct
tgctaaattaggtgaaggctagctacaacgatgtccata

SEQ ID NO: 138 - DNA, 39 bp, Artificial Sequence
  Synthetic Construct
taaacagtgttattaaggctagctacaacgagatagaaa

SEQ ID NO: 139 - DNA, 39 bp, Artificial Sequence
  Synthetic Construct
ttttattttcaaacaaggctagctacaacgatctacatc

SEQ ID NO: 140 - DNA, 39 bp, Artificial Sequence
  Synthetic Construct
ttattgagtattttcaggctagctacaacgactctggta

SEQ ID NO: 141 - DNA, 39 bp, Artificial Sequence
  Synthetic Construct
tatatgtgctggagcaggctagctacaacgactcttttg

SEQ ID NO: 142 - DNA, 39 bp, Artificial Sequence
  Synthetic Construct
atagaacaaacaccaaggctagctacaacgaagtagata

SEQ ID NO: 143 - DNA, 39 bp, Artificial Sequence
  Synthetic Construct
gtgagtggtgcacaaaggctagctacaacgacgtttcag

SEQ ID NO: 144 - DNA, 39 bp, Artificial Sequence
  Synthetic Construct
tgtgactccattaagaggctagctacaacgatagcttgt

SEQ ID NO: 145 - DNA, 39 bp, Artificial Sequence
  Synthetic Construct
ttgaactgtgtttttaggctagctacaacgaggcttctc

SEQ ID NO: 146 - DNA, 39 bp, Artificial Sequence
  Synthetic Construct
accatcaactttcttaggctagctacaacgaaataattg

SEQ ID NO: 147 - DNA, 39 bp, Artificial Sequence
  Synthetic Construct
agtaagtttcaggtaaggctagctacaacgatgttggac

SEQ ID NO: 148 - DNA, 39 bp, Artificial Sequence
  Synthetic Construct
tttaaattcttgtaaaggctagctacaacgattctactc

SEQ ID NO: 149 - DNA, 39 bp, Artificial Sequence
  Synthetic Construct
aattctaagaaatcaaggctagctacaacgattccattt

SEQ ID NO: 150 - DNA, 39 bp, Artificial Sequence
  Synthetic Construct
ccgttcaatgaattcaggctagctacaacgaccatagct

SEQ ID NO: 151 - DNA, 39 bp, Artificial Sequence
  Synthetic Construct
gaataaaatcttctaaggctagctacaacgatcaaaagg

SEQ ID NO: 152 - DNA, 39 bp, Artificial Sequence
  Synthetic Construct
gtactgtccataggaaggctagctacaacgaaaaatctt

SEQ ID NO: 153 - DNA, 39 bp, Artificial Sequence
  Synthetic Construct
caaaatcatcaagtaaggctagctacaacgaaaatcaat

SEQ ID NO: 154 - DNA, 39 bp, Artificial Sequence
  Synthetic Construct
tgatgttgatatgacaggctagctacaacgaggtcgtaa

SEQ ID NO: 155 - DNA, 39 bp, Artificial Sequence
  Synthetic Construct
cttgtccattagcacaggctagctacaacgaaatggaaa

SEQ ID NO: 156 - DNA, 39 bp, Artificial Sequence
  Synthetic Construct
ttatataaaccaaaaaggctagctacaacgattgtccat

SEQ ID NO: 157 - DNA, 39 bp, Artificial Sequence
  Synthetic Construct
aatgtaatcaccagcaggctagctacaacgattgtccag

SEQ ID NO: 158 - DNA, 39 bp, Artificial Sequence
  Synthetic Construct
aacttcccatgaaagaggctagctacaacgagtaattct

SEQ ID NO: 159 - DNA, 39 bp, Artificial Sequence
  Synthetic Construct
aatcaccaacatttaaggctagctacaacgattgtaagt

SEQ ID NO: 160 - DNA, 39 bp, Artificial Sequence
  Synthetic Construct
gtatgtgatgtcagcaggctagctacaacgaaaaataat

SEQ ID NO: 161 - DNA, 39 bp, Artificial Sequence
  Synthetic Construct
taatggcattactgtaggctagctacaacgagtgatgtc

SEQ ID NO: 162 - DNA, 39 bp, Artificial Sequence
  Synthetic Construct
gggtataagccagtaaggctagctacaacgatctaacat

SEQ ID NO: 163 - DNA, 39 bp, Artificial Sequence
  Synthetic Construct
tttatctataggcaaaggctagctacaacgaattttaat

SEQ ID NO: 164 - DNA, 39 bp, Artificial Sequence
  Synthetic Construct
tcatcaaagacaactaggctagctacaacgaatctgctg

SEQ ID NO: 165 - DNA, 39 bp, Artificial Sequence
  Synthetic Construct
aacactcaaatcataaggctagctacaacgattgtggcc

SEQ ID NO: 166 - DNA, 39 bp, Artificial Sequence
  Synthetic Construct
agtgcttagcacgtaaggctagctacaacgactggcatt

SEQ ID NO: 167 - DNA, 39 bp, Artificial Sequence
  Synthetic Construct
acacactgaattgaaaggctagctacaacgaattctggt

SEQ ID NO: 168 - DNA, 39 bp, Artificial Sequence
  Synthetic Construct
ggacctatagttttcaggctagctacaacgaaagtctac

SEQ ID NO: 169 - DNA, 39 bp, Artificial Sequence
  Synthetic Construct
aacaatttcagcaggaggctagctacaacgaaacgccga

SEQ ID NO: 170 - DNA, 39 bp, Artificial Sequence
  Synthetic Construct
taacacccttataaaaggctagctacaacgaattttaaa

SEQ ID NO: 171 - DNA, 39 bp, Artificial Sequence
  Synthetic Construct
tctcttaccacgcctaggctagctacaacgattgtggcc

SEQ ID NO: 172 - DNA, 39 bp, Artificial Sequence
  Synthetic Construct
ggttacgtgtaaggaaggctagctacaacgatctcttac

SEQ ID NO: 173 - DNA, 39 bp, Artificial Sequence
  Synthetic Construct
agcattctgtgaattaggctagctacaacgaaaggtgaa

SEQ ID NO: 174 - DNA, 39 bp, Artificial Sequence
  Synthetic Construct
aaccttcagttttgaaggctagctacaacgattagtgtc

SEQ ID NO: 175 - DNA, 39 bp, Artificial Sequence
  Synthetic Construct
caggtatgtcaacacaggctagctacaacgaaaaccttc

SEQ ID NO: 176 - DNA, 39 bp, Artificial Sequence
  Synthetic Construct
ttagggtaaccattaaggctagctacaacgattgataat

SEQ ID NO: 177 - DNA, 39 bp, Artificial Sequence
  Synthetic Construct
gcttcttcgcgggtgaggctagctacaacgaaaacatgt

SEQ ID NO: 178 - DNA, 39 bp, Artificial Sequence
  Synthetic Construct
cctgtaggtacagcaaggctagctacaacgataggttaa

SEQ ID NO: 179 - DNA, 39 bp, Artificial Sequence
  Synthetic Construct
gaggtgtttaaattgaggctagctacaacgactccaggc

SEQ ID NO: 180 - DNA, 39 bp, Artificial Sequence
  Synthetic Construct
aaggaagtcctttgtaggctagctacaacgaataagtgg

SEQ ID NO: 181 - DNA, 39 bp, Artificial Sequence
  Synthetic Construct
cttaacatttgtacaaggctagctacaacgactttatac

SEQ ID NO: 182 - DNA, 39 bp, Artificial Sequence
  Synthetic Construct
gcccataagacaaataggctagctacaacgagactctgt

SEQ ID NO: 183 - DNA, 39 bp, Artificial Sequence
  Synthetic Construct
gtgcgctcaggtcctaggctagctacaacgatttcacaa

SEQ ID NO: 184 - DNA, 39 bp, Artificial Sequence
  Synthetic Construct
gttgctttgtaggttaggctagctacaacgactgtaaaa

SEQ ID NO: 185 - DNA, 39 bp, Artificial Sequence
  Synthetic Construct
accatggacttgacaaggctagctacaacgaacagatca

SEQ ID NO: 186 - DNA, 39 bp, Artificial Sequence
  Synthetic Construct
attataggatattcaaggctagctacaacgaagtccagt

SEQ ID NO: 187 - DNA, 39 bp, Artificial Sequence
  Synthetic Construct
attaatcttcagttcaggctagctacaacgacaccaatt

SEQ ID NO: 188 - DNA, 39 bp, Artificial Sequence
  Synthetic Construct
acaaccatgtgttgaaggctagctacaacgactttctac

SEQ ID NO: 189 - DNA, 39 bp, Artificial Sequence
  Synthetic Construct
gctttagggttaccaaggctagctacaacgagtcgtgaa

SEQ ID NO: 190 - DNA, 39 bp, Artificial Sequence
  Synthetic Construct
aattccaaaataggcaggctagctacaacgaacaccatc

SEQ ID NO: 191 - DNA, 39 bp, Artificial Sequence
  Synthetic Construct
aacaaaagcacttttaggctagctacaacgacaaaagct

SEQ ID NO: 192 - DNA, 39 bp, Artificial Sequence
  Synthetic Construct
tggactgtcagagtaaggctagctacaacgaagaaaaat

SEQ ID NO: 193 - DNA, 39 bp, Artificial Sequence
  Synthetic Construct
cttgttttccatgagaggctagctacaacgatcacatgg

SEQ ID NO: 194 - DNA, 39 bp, Artificial Sequence
  Synthetic Construct
gggagtgaggcttgtaggctagctacaacgacggtatcg

SEQ ID NO: 195 - DNA, 39 bp, Artificial Sequence
  Synthetic Construct
cgccaacaataagccaggctagctacaacgaccgaaagg

SEQ ID NO: 196 - DNA, 39 bp, Artificial Sequence
  Synthetic Construct
acagcaagaagtgcaaggctagctacaacgagccaacaa

SEQ ID NO: 197 - DNA, 39 bp, Artificial Sequence
  Synthetic Construct
gaagcgctctgaaaaaggctagctacaacgaagcaagaa

SEQ ID NO: 198 - DNA, 39 bp, Artificial Sequence
  Synthetic Construct
agagtgctagttgccaggctagctacaacgactcttttt

SEQ ID NO: 199 - DNA, 39 bp, Artificial Sequence
  Synthetic Construct
ttgcaaacaaagtgaaggctagctacaacgaacccttgg

SEQ ID NO: 200 - DNA, 39 bp, Artificial Sequence
  Synthetic Construct
aaactgttacaaacaaggctagctacaacgaaacagcaa

SEQ ID NO: 201 - DNA, 39 bp, Artificial Sequence
  Synthetic Construct
aaaaggtgtgagtaaaggctagctacaacgatgttacaa

SEQ ID NO: 202 - DNA, 39 bp, Artificial Sequence
  Synthetic Construct
caaagccaaagcctcaggctagctacaacgatattattc

SEQ ID NO: 203 - DNA, 39 bp, Artificial Sequence
  Synthetic Construct
tggcatcataaagtaaggctagctacaacgagggttttt

SEQ ID NO: 204 - DNA, 39 bp, Artificial Sequence
  Synthetic Construct
atgccagcaaagaaaaggctagctacaacgaagttggca

SEQ ID NO: 205 - DNA, 39 bp, Artificial Sequence
  Synthetic Construct
acaatagtcgtaacaaggctagctacaacgatagtatgc

SEQ ID NO: 206 - DNA, 39 bp, Artificial Sequence
  Synthetic Construct
accaatctggtagtcaggctagctacaacgagttcagaa

SEQ ID NO: 207 - DNA, 39 bp, Artificial Sequence
  Synthetic Construct
ttactccagattcccaggctagctacaacgattttcagt

SEQ ID NO: 208 - DNA, 39 bp, Artificial Sequence
  Synthetic Construct
gatgaagaaggtaacaggctagctacaacgagttcaaca

SEQ ID NO: 209 - DNA, 39 bp, Artificial Sequence
  Synthetic Construct
attactggattaacaaggctagctacaacgatccggatg

SEQ ID NO: 210 - DNA, 39 bp, Artificial Sequence
  Synthetic Construct
aagccacgtacgagcaggctagctacaacgagtcgcgaa

SEQ ID NO: 211 - DNA, 39 bp, Artificial Sequence
  Synthetic Construct
cgtgcctctgataagaggctagctacaacgactcctcca

SEQ ID NO: 212 - DNA, 39 bp, Artificial Sequence
  Synthetic Construct
cctttttcaacttctaggctagctacaacgataagccac

SEQ ID NO: 213 - DNA, 39 bp, Artificial Sequence
  Synthetic Construct
acgtttgatgaacacaggctagctacaacgaagggctgt

SEQ ID NO: 214 - DNA, 39 bp, Artificial Sequence
  Synthetic Construct
agttcgagcatccgaaggctagctacaacgagtttgatg

SEQ ID NO: 215 - DNA, 39 bp, Artificial Sequence
  Synthetic Construct
aaccataacatgaccaggctagctacaacgagaggtgca

SEQ ID NO: 216 - DNA, 39 bp, Artificial Sequence
  Synthetic Construct
tgtctcaccactacgaggctagctacaacgacgtactga

SEQ ID NO: 217 - DNA, 39 bp, Artificial Sequence
  Synthetic Construct
atgagggacaaggacaggctagctacaacgacaagtgtc

SEQ ID NO: 218 - DNA, 39 bp, Artificial Sequence
  Synthetic Construct
gccaccagctcctttaggctagctacaacgataccgttc

SEQ ID NO: 219 - DNA, 39 bp, Artificial Sequence
  Synthetic Construct
cgcctaagtcaaatgaggctagctacaacgatttagatc

SEQ ID NO: 220 - DNA, 39 bp, Artificial Sequence
  Synthetic Construct
ttcacgggtaacaccaggctagctacaacgatgctatgt

SEQ ID NO: 221 - DNA, 39 bp, Artificial Sequence
  Synthetic Construct
cacttttctaatagcaggctagctacaacgatctttgca

SEQ ID NO: 222 - DNA, 39 bp, Artificial Sequence
  Synthetic Construct
aattttgaaggtcacaggctagctacaacgattttctaa

SEQ ID NO: 223 - DNA, 39 bp, Artificial Sequence
  Synthetic Construct
tttaggtaatgttgcaggctagctacaacgatatcacca

SEQ ID NO: 224 - DNA, 39 bp, Artificial Sequence
  Synthetic Construct
tgagtatattttgcgaggctagctacaacgaattcatca

SEQ ID NO: 225 - DNA, 39 bp, Artificial Sequence
  Synthetic Construct
tgttaatgtgtttaaaggctagctacaacgaattgacac

SEQ ID NO: 226 - DNA, 39 bp, Artificial Sequence
  Synthetic Construct
aaatgtataactctcaggctagctacaacgaattatagg

SEQ ID NO: 227 - DNA, 39 bp, Artificial Sequence
  Synthetic Construct
tggtgcaactcctttaggctagctacaacgacagaacca

SEQ ID NO: 228 - DNA, 39 bp, Artificial Sequence
  Synthetic Construct
gacaaagtcattaagaggctagctacaacgactgaatcg

SEQ ID NO: 229 - DNA, 39 bp, Artificial Sequence
  Synthetic Construct
gttgcacaatcaccaaggctagctacaacgacaaagttg

SEQ ID NO: 230 - DNA, 39 bp, Artificial Sequence
  Synthetic Construct
accctctttagagtcaggctagctacaacgatttctttt

SEQ ID NO: 231 - DNA, 39 bp, Artificial Sequence
  Synthetic Construct
gctagcttttgttgtaggctagctacaacgaaaacccac

SEQ ID NO: 232 - DNA, 39 bp, Artificial Sequence
  Synthetic Construct
tgtataaacccacaaaggctagctacaacgagtaagtga

SEQ ID NO: 233 - DNA, 39 bp, Artificial Sequence
  Synthetic Construct
gcattcacattagtaaggctagctacaacgaaaaggctg

SEQ ID NO: 234 - DNA, 39 bp, Artificial Sequence
  Synthetic Construct
gcgtggtttgccaagaggctagctacaacgaaattacat

SEQ ID NO: 235 - DNA, 39 bp, Artificial Sequence
  Synthetic Construct
atgacataaccatctaggctagctacaacgattgttcgc

SEQ ID NO: 236 - DNA, 39 bp, Artificial Sequence
  Synthetic Construct
cctccaaaatatgtaaggctagctacaacgattgcatgc

SEQ ID NO: 237 - DNA, 39 bp, Artificial Sequence
  Synthetic Construct
ttgtattcctccaaaaggctagctacaacgaatgtaatt

SEQ ID NO: 238 - DNA, 39 bp, Artificial Sequence
  Synthetic Construct
atttactcatgtcaaaggctagctacaacgaaaagaata

SEQ ID NO: 239 - DNA, 39 bp, Artificial Sequence
  Synthetic Construct
agaagagataaaatcaggctagctacaacgaatcattga

SEQ ID NO: 240 - DNA, 39 bp, Artificial Sequence
  Synthetic Construct
ctcaagagggtagccaggctagctacaacgacagggcca

SEQ ID NO: 241 - DNA, 39 bp, Artificial Sequence
  Synthetic Construct
gctagaaggtctttaaggctagctacaacgagcactcaa

SEQ ID NO: 242 - DNA, 39 bp, Artificial Sequence
  Synthetic Construct
agtccagttgttcggaggctagctacaacgaaaagtgca

SEQ ID NO: 243 - DNA, 39 bp, Artificial Sequence
  Synthetic Construct
caaaaggtgtctgcaaggctagctacaacgatcatagct

SEQ ID NO: 244 - DNA, 39 bp, Artificial Sequence
  Synthetic Construct
cattgaaggtgtcaaaggctagctacaacgattctttgc

SEQ ID NO: 245 - DNA, 39 bp, Artificial Sequence
  Synthetic Construct
taagggaaatacaaaaggctagctacaacgattggacat

SEQ ID NO: 246 - DNA, 39 bp, Artificial Sequence
  Synthetic Construct
caactggatagacagaggctagctacaacgacgaattct

SEQ ID NO: 247 - DNA, 39 bp, Artificial Sequence
  Synthetic Construct
tgagagttgaaaggcaggctagctacaacgaatttggtt

SEQ ID NO: 248 - DNA, 39 bp, Artificial Sequence
  Synthetic Construct
ccatgaagtttcaccaggctagctacaacgaaatgatca

SEQ ID NO: 249 - DNA, 39 bp, Artificial Sequence
  Synthetic Construct
accttctttagtcaaaggctagctacaacgatctcagtg

SEQ ID NO: 250 - DNA, 39 bp, Artificial Sequence
  Synthetic Construct
attttggggtaagtaaggctagctacaacgacacaagta

SEQ ID NO: 251 - DNA, 39 bp, Artificial Sequence
  Synthetic Construct
catgctggacaataaaggctagctacaacgatttaacaa

SEQ ID NO: 252 - DNA, 39 bp, Artificial Sequence
  Synthetic Construct
tttcaagccagattcaggctagctacaacgatatggtat

SEQ ID NO: 253 - DNA, 39 bp, Artificial Sequence
  Synthetic Construct
cagcctccaaaggcaaggctagctacaacgaagtgcgac

SEQ ID NO: 254 - DNA, 39 bp, Artificial Sequence
  Synthetic Construct
aaggttgtcattaagaggctagctacaacgacttcggaa

SEQ ID NO: 255 - DNA, 39 bp, Artificial Sequence
  Synthetic Construct
agtttccacaaaagcaggctagctacaacgattgtggaa

SEQ ID NO: 256 - DNA, 39 bp, Artificial Sequence
  Synthetic Construct
caacaatttgtttgaaggctagctacaacgagctttata

SEQ ID NO: 257 - DNA, 39 bp, Artificial Sequence
  Synthetic Construct
gcttttccttttgtaaggctagctacaacgatttaaaat

SEQ ID NO: 258 - DNA, 39 bp, Artificial Sequence
  Synthetic Construct
gaggactcagtattgaggctagctacaacgattctgttc

SEQ ID NO: 259 - DNA, 39 bp, Artificial Sequence
  Synthetic Construct
ttctgtaaaacacgcaggctagctacaacgaagaatttt

SEQ ID NO: 260 - DNA, 39 bp, Artificial Sequence
  Synthetic Construct
ccaaatcagatgtgaaggctagctacaacgaatcatagc

SEQ ID NO: 261 - DNA, 39 bp, Artificial Sequence
  Synthetic Construct
gggtttgagtttttcaggctagctacaacgaaaacagtg

SEQ ID NO: 262 - DNA, 39 bp, Artificial Sequence
  Synthetic Construct
ctacaccttccttaaaggctagctacaacgattctcttc

SEQ ID NO: 263 - DNA, 39 bp, Artificial Sequence
  Synthetic Construct
acaatttgtccaccgaggctagctacaacgaaatttcac

SEQ ID NO: 264 - DNA, 39 bp, Artificial Sequence
  Synthetic Construct
tgaacactctccttaaggctagctacaacgattcctttg

SEQ ID NO: 265 - DNA, 39 bp, Artificial Sequence
  Synthetic Construct
aaatgtttcacctaaaggctagctacaacgatcaaggct

SEQ ID NO: 266 - DNA, 39 bp, Artificial Sequence
  Synthetic Construct
tcttctctggatttaaggctagctacaacgaacactttc

SEQ ID NO: 267 - DNA, 39 bp, Artificial Sequence; identical to SEQ ID NO: 266
  Synthetic Construct

SEQ ID NO: 268 - DNA, 39 bp, Artificial Sequence
  Synthetic Construct
aaactcttcttcttcaggctagctacaacgaaatcacct

SEQ ID NO: 269 - DNA, 39 bp, Artificial Sequence
  Synthetic Construct
tttaccttggtaatcaggctagctacaacgacttcagta

SEQ ID NO: 270 - DNA, 39 bp, Artificial Sequence
  Synthetic Construct
ttgttgactatcatcaggctagctacaacgactaaccaa

SEQ ID NO: 271 - DNA, 39 bp, Artificial Sequence
  Synthetic Construct
gcatttttaatgtataggctagctacaacgaattgtcag

SEQ ID NO: 272 - DNA, 39 bp, Artificial Sequence
  Synthetic Construct
accactgttggttttaggctagctacaacgactttttag

SEQ ID NO: 273 - DNA, 39 bp, Artificial Sequence
  Synthetic Construct
tcagattcaacttgcaggctagctacaacgaggcattgt

SEQ ID NO: 274 - DNA, 39 bp, Artificial Sequence
  Synthetic Construct
attagtagctatgtaaggctagctacaacgacatcagat

SEQ ID NO: 275 - DNA, 39 bp, Artificial Sequence
  Synthetic Construct
ctcttaagaagttgaaggctagctacaacgagtcttcac

SEQ ID NO: 276 - DNA, 39 bp, Artificial Sequence
  Synthetic Construct
tagaacttcgtgctgaggctagctacaacgataaaattt

SEQ ID NO: 277 - DNA, 39 bp, Artificial Sequence
  Synthetic Construct
taccagctgataataaggctagctacaacgaggtgcaag

SEQ ID NO: 278 - DNA, 39 bp, Artificial Sequence
  Synthetic Construct
acagtatctacacaaaggctagctacaacgatcttaaag

SEQ ID NO: 279 - DNA, 39 bp, Artificial Sequence
  Synthetic Construct
aagacagctaagtagaggctagctacaacgaatttgtgc

SEQ ID NO: 280 - DNA, 39 bp, Artificial Sequence
  Synthetic Construct
tgaaacaagtttgtcaggctagctacaacgaagagattt

SEQ ID NO: 281 - DNA, 39 bp, Artificial Sequence
  Synthetic Construct
ggtttactttcagttaggctagctacaacgaaaatggct

SEQ ID NO: 282 - DNA, 39 bp, Artificial Sequence
  Synthetic Construct
tcaacacaagctttgaggctagctacaacgatttcttat

SEQ ID NO: 283 - DNA, 39 bp, Artificial Sequence
  Synthetic Construct
tggatgaagattgccaggctagctacaacgataatgtca

SEQ ID NO: 284 - DNA, 39 bp, Artificial Sequence
  Synthetic Construct
atgtcaatgtcactaaggctagctacaacgaaagagtgg

SEQ ID NO: 285 - DNA, 39 bp, Artificial Sequence
  Synthetic Construct
catcacccactatataggctagctacaacgaggagcatc

SEQ ID NO: 286 - DNA, 39 bp, Artificial Sequence
  Synthetic Construct
accacagcagttaaaaggctagctacaacgaaccctctt

SEQ ID NO: 287 - DNA, 39 bp, Artificial Sequence
  Synthetic Construct
cgggtaagtggttataggctagctacaacgaaattgtct

SEQ ID NO: 288 - DNA, 39 bp, Artificial Sequence
  Synthetic Construct
ctctacagtgtaaccaggctagctacaacgattaaaccc

SEQ ID NO: 289 - DNA, 39 bp, Artificial Sequence
  Synthetic Construct
ttacactttttaagcaggctagctacaacgatgtctttg

SEQ ID NO: 290 - DNA, 39 bp, Artificial Sequence
  Synthetic Construct
tagaatgtaaaaggcaggctagctacaacgattttacac

SEQ ID NO: 291 - DNA, 39 bp, Artificial Sequence
  Synthetic Construct
tgcttctcattagagaggctagctacaacgaaatagatg

SEQ ID NO: 292 - DNA, 39 bp, Artificial Sequence
  Synthetic Construct
aagcatttctcgcaaaggctagctacaacgatccaagaa

SEQ ID NO: 293 - DNA, 39 bp, Artificial Sequence
  Synthetic Construct
tggtgtaaaagtaaaaggctagctacaacgactagcacc

SEQ ID NO: 294 - DNA, 39 bp, Artificial Sequence
  Synthetic Construct
tgtaacaagagtttcaggctagctacaacgattagatcg

SEQ ID NO: 295 - DNA, 39 bp, Artificial Sequence
  Synthetic Construct
agaagaagaagtaagaggctagctacaacgaaaccatta

SEQ ID NO: 296 - DNA, 673 bp, SARS-CoV-2
gggatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaag

SEQ ID NO: 297 - DNA, 921 bp, SARS-CoV-2
gggagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaa

SEQ ID NO: 298 - DNA, 844 bp, SARS-CoV-2
gggtcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagc

SEQ ID NO: 299 - DNA, 584 bp, SARS-CoV-2
gggcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaa

SEQ ID NO: 300 - DNA, 1042 bp, SARS-CoV-2
gggagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaat

SEQ ID NO: 301 - DNA, 890 bp, SARS-CoV-2
gggtctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgt

SEQ ID NO: 302 - DNA, 1805 bp, SARS-CoV-2
gggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaa

SEQ ID NO: 303 - DNA, 1583 bp, SARS-CoV-2
gggctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcag

SEQ ID NO: 304 - DNA, 831 bp, SARS-CoV-2
gggatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaa

SEQ ID NO: 305 - DNA, 543 bp, SARS-CoV-2
gggatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggg

SEQ ID NO: 306 - DNA, 1916 bp, SARS-CoV-2
gggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggt

SEQ ID NO: 307 - DNA, 1798 bp, SARS-CoV-2
gggctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctgg

SEQ ID NO: 308 - DNA, 51 bp, Artificial Sequence
  Synthetic Construct
tccccatttattataggcattaacaatgaatgttagagtttttcattagga

SEQ ID NO: 309 - DNA, 51 bp, Artificial Sequence
  Synthetic Construct
tccccatttattaatttttgatgaaactgtcgttagagtttttcattagga

SEQ ID NO: 310 - DNA, 51 bp, Artificial Sequence
  Synthetic Construct
tccccatttatctacagtagctcctctagtggttagagtttttcattagga

SEQ ID NO: 311 - DNA, 51 bp, Artificial Sequence
  Synthetic Construct
tccccatttattaaggtgagggttttctacagttagagtttttcattagga

SEQ ID NO: 312 - DNA, 51 bp, Artificial Sequence
  Synthetic Construct
tccccatttatccatttcactcaatacttgagttagagtttttcattagga

SEQ ID NO: 313 - DNA, 51 bp, Artificial Sequence
  Synthetic Construct
tccccatttatccacatgaaccattaaggaagttagagtttttcattagga

SEQ ID NO: 314 - DNA, 51 bp, Artificial Sequence
  Synthetic Construct
tccccatttattgaggtgcagttcgagcatcgttagagtttttcattagga

SEQ ID NO: 315 - DNA, 51 bp, Artificial Sequence
  Synthetic Construct
tccccatttattaaacaccaagagtcagtctgttagagtttttcattagga

SEQ ID NO: 316 - DNA, 51 bp, Artificial Sequence
  Synthetic Construct
tccccatttatctttttaagaacaacttcaggttagagtttttcattagga

SEQ ID NO: 317 - DNA, 51 bp, Artificial Sequence
  Synthetic Construct
tccccatttattagcttgatcagccatctttgttagagtttttcattagga

SEQ ID NO: 318 - DNA, 51 bp, Artificial Sequence
  Synthetic Construct
tccccatttattagcactagtaacttttgccgttagagtttttcattagga

SEQ ID NO: 319 - DNA, 51 bp, Artificial Sequence
  Synthetic Construct
tccccatttattagtctggtatgacaaccatgttagagtttttcattagga

SEQ ID NO: 320 - DNA, 51 bp, Artificial Sequence
  Synthetic Construct
tccccatttatttgtccatactaatttcactgttagagtttttcattagga

SEQ ID NO: 321 - DNA, 51 bp, Artificial Sequence
  Synthetic Construct
tccccatttatcggcttctccaattaatgtggttagagtttttcattagga

SEQ ID NO: 322 - DNA, 51 bp, Artificial Sequence
  Synthetic Construct
tccccatttatttcaaaaggtgattccttaagttagagtttttcattagga

SEQ ID NO: 323 - DNA, 51 bp, Artificial Sequence
  Synthetic Construct
tccccatttattttgtccagtcacatgttgcgttagagtttttcattagga

SEQ ID NO: 324 - DNA, 51 bp, Artificial Sequence
  Synthetic Construct
tccccatttattgtaattctctgtcagacaggttagagtttttcattagga

SEQ ID NO: 325 - DNA, 51 bp, Artificial Sequence
  Synthetic Construct
tccccatttatcaaaataatcaccaacatttgttagagtttttcattagga

SEQ ID NO: 326 - DNA, 51 bp, Artificial Sequence
  Synthetic Construct
tccccatttattaagtctacacactgaattggttagagtttttcattagga

SEQ ID NO: 327 - DNA, 51 bp, Artificial Sequence
  Synthetic Construct
tccccatttattctccaggcggtggtttagcgttagagtttttcattagga

SEQ ID NO: 328 - DNA, 51 bp, Artificial Sequence
  Synthetic Construct
tccccatttatcgactctgtcagagagatttgttagagtttttcattagga

SEQ ID NO: 329 - DNA, 51 bp, Artificial Sequence
  Synthetic Construct
tccccatttatcctttctacaagccgcattagttagagtttttcattagga

SEQ ID NO: 330 - DNA, 51 bp, Artificial Sequence
  Synthetic Construct
tccccatttattgtcgtgaagaactgggaatgttagagtttttcattagga

SEQ ID NO: 331 - DNA, 51 bp, Artificial Sequence
  Synthetic Construct
tccccatttatctcacatggactgtcagagtgttagagtttttcattagga

SEQ ID NO: 332 - DNA, 51 bp, Artificial Sequence
  Synthetic Construct
tccccatttatttctcagtgccacaaaattcgttagagtttttcattagga

SEQ ID NO: 333 - DNA, 51 bp, Artificial Sequence
  Synthetic Construct
tccccatttatcagaattttgagcagtttcagttagagtttttcattagga

SEQ ID NO: 334 - DNA, 51 bp, Artificial Sequence
  Synthetic Construct
tccccatttatcttctcttcaagccaatcaagttagagtttttcattagga

SEQ ID NO: 335 - DNA, 51 bp, Artificial Sequence
  Synthetic Construct
tccccatttatcacactttctgtacaatcccgttagagtttttcattagga

SEQ ID NO: 336 - DNA, 51 bp, Artificial Sequence
  Synthetic Construct
tccccatttatcaatcaccttcttcttcatcgttagagtttttcattagga

SEQ ID NO: 337 - DNA, 51 bp, Artificial Sequence
  Synthetic Construct
tccccatttattggtgcaagtagaacttcgtgttagagtttttcattagga

SEQ ID NO: 338 - DNA, 51 bp, Artificial Sequence
  Synthetic Construct
tccccatttatcaagagtggcagaatctggagttagagtttttcattagga

SEQ ID NO: 339 - DNA, 51 bp, Artificial Sequence
  Synthetic Construct
tccccatttattggagcatctttctttaagagttagagtttttcattagga

SEQ ID NO: 340 - DNA, 51 bp, Artificial Sequence
  Synthetic Construct
tccccatttatcaccctcttgaacaacatcagttagagtttttcattagga

SEQ ID NO: 341 - DNA, 51 bp, Artificial Sequence
  Synthetic Construct
tccccatttatttttcttttgtaacatttttgttagagtttttcattagga

SEQ ID NO: 342 - DNA, 51 bp, Artificial Sequence
  Synthetic Construct
tccccatttattttgcatgcatgacataaccgttagagtttttcattagga

SEQ ID NO: 343 - DNA, 60 bp, Artificial Sequence
  Synthetic Construct
cgtaatgcggggtgcatttcgggatcctgtttgtaatcagttcctcttttggtgtattca

SEQ ID NO: 344 - DNA, 65 bp, Artificial Sequence
  Synthetic Construct
cgtaatgcggggtgcatttcgctgatggatcctgtttgtaatcagttcctcttttggtgtattca

SEQ ID NO: 345 - DNA, 70 bp, Artificial Sequence
  Synthetic Construct
cgtaatgcggggtgcatttcgctgattttggggatcctgtttgtaatcagttcctcttttggtgtattca

SEQ ID NO: 346 - DNA, 75 bp, Artificial Sequence
  Synthetic Construct
cgtaatgcggggtgcatttcgctgattttggggtccggatcctgtttgtaatcagttcctcttttggtgtattca

SEQ ID NO: 347 - DNA, 83 bp, Artificial Sequence
  Synthetic Construct
cgtaatgcggggtgcttaaaaagacagtaggtactcattaggatcctgtttgtaatcagttcctttttcttttggtgtattca

SEQ ID NO: 348 - DNA, 83 bp, Artificial Sequence
  Synthetic Construct
tgaatacaccaaaagaaaaaggaactgattacaaacaggatcctaatgagtacctactgtctttttaagcaccccgcattacg

SEQ ID NO: 349 - DNA, 22 bp, Artificial Sequence
  Synthetic Construct
ccgcattacgtgaatacaccaa

SEQ ID NO: 350 - DNA, 50 bp, Artificial Sequence
  Synthetic Construct
  misc_feature (50)..(50): n contains an inverted dT
ctaatgagtacctactgtctaaaaaaaactggatgatcctatgaactgan

SEQ ID NO: 351 - DNA, 42 bp, Artificial Sequence
  Synthetic Construct
  misc_feature (42)..(42): n contains an inverted dT
tttttagacagtaggtactcattaggatcctgtttgtaatcn

SEQ ID NO: 352 - DNA, 31 bp, Artificial Sequence
  Synthetic Construct
  misc_feature (31)..(31): n is thiol
agacagtaggtactcattagttttttttttn

SEQ ID NO: 353 - DNA, 35 bp, Artificial Sequence
  Synthetic Construct
  misc_feature (1)..(1): n is biotin
ntttttttttttagtcagttcataggatcatccag

SEQ ID NO: 354 - DNA, 33 bp, Artificial Sequence
  Synthetic Construct
  misc_feature (33)..(33): n is thiol
acctgggggagtattgcggaggaaggttttttn

SEQ ID NO: 355 - DNA, 33 bp, Artificial Sequence
  Synthetic Construct
  misc_feature (33)..(33): n is biotin
accttcctccgcaatactcccccaggttttttn

SEQ ID NO: 356 - DNA, 91 bp, Artificial Sequence
  Synthetic Construct
  misc_feature (1)..(1): n is phosphate
ngggtccattatcagacatcctcagctttttagacagtaggtactcattaggatcctgtttgtaatccctcagcgcatttcgctgattttg

SEQ ID NO: 357 - DNA, 18 bp, Artificial Sequence
  Synthetic Construct
acctactgtctaaaaagc

SEQ ID NO: 358 - DNA, 93 bp, Artificial Sequence
  Synthetic Construct
gaatctgagggtccaccaaacgtatcctcagcttcagttcataggatcatccagaaaaaaaagacagtaggtactcattagttcctcagctca

SEQ ID NO: 359 - DNA, 31 bp, Artificial Sequence
  Synthetic Construct
tggaccctcagattctgagctgaggaactaa

SEQ ID NO: 360 - DNA, 91 bp, Artificial Sequence
  Synthetic Construct
ctgccagttgaatctgagggtctcctcagcttcagttcataggatcatccagaaaaaaaagacagtaggtactcattagttcctcagctca

SEQ ID NO: 361 - DNA, 31 bp, Artificial Sequence
  Synthetic Construct
agattcaactggcagtgagctgaggaactaa